(19) United States
(12) Patent Application Publication
(10) Pub. No.: US 2025/0331529 A1
(43) Pub. Date: Oct. 30, 2025

(54) Method of Producing a Milk-Based Product

(71) Applicant: Novozymes A/S, Bagsvaerd (DK)

(72) Inventors: Jeppe Wegener TAMS, Gentofte (DK); Ali OSMAN, Sabro (DK); Nikolaj SPODSBERG, Holte (DK); Jesper SALOMON, Holte (DK); Julie Bille RANNES, Hoersholm (DK); Jan Kjoelhede VESTER, Hvidovre (DK); Frank Winther RASMUSSEN, Roskilde (DK); Annette Helle JOHANSEN, Bronshoj (DK); Roland Alexander PACHE, Valby (DK)

(73) Assignee: Novozymes A/S, Bagsvaerd (DK)

(21) Appl. No.: 18/720,852

(22) PCT Filed: Dec. 22, 2022
(86) PCT No.: PCT/EP2022/087482

§ 371 Date: Jun. 17, 2024

(30) Foreign Application Priority Data
Dec. 22, 2021 (EP) 21216998.1

(51) Int. Cl. A23C 9/12; A23B 11/13

(52) U.S. Cl. CPC A23C 9/1206; A23B 11/13; C12Y 302/01108

(57) ABSTRACT

The present invention relates to a method of producing a lactose-reduced heat-treated milk-based product which comprises treatment of a milk-based substrate with a lactase and performing a heat treatment.

### REFERENCE TO SEQUENCE LISTING

This application contains a Sequence Listing in computer readable form. The computer readable form is incorporated herein by reference.

### FIELD OF THE INVENTION

The present invention relates to a method of producing a lactose-reduced heat-treated milk-based product which comprises treatment of a milk-based substrate with a lactase and performing a heat treatment.

### BACKGROUND OF THE INVENTION

Most lactose-reduced or lactose-free milk-based products are produced in batch processes, i.e. adding lactase into milk and then incubating the milk at cold temperatures (usually less than 10° C.) for enough time to reduce the lactose content to less than 0.01% or less than 0.1% (which in most countries allows for labelling of, e.g., milk as lactose-free), followed by heat treatment such as pasteurization, UHT or ESL (extended shelf-life of up to 35 days) treatments. Some of the drawbacks linked to the batch application of lactases include:
(1) the incubation time can be up to 24 h and sometimes more, which raises concerns regarding capacity and capital investments in new incubation tanks to respond to the growing demand of lactose free dairy products.
(2) Prolonged incubation times give the chance to psychotropic microbes to grow and secrete their enzymes (particularly proteases), of which some are heat stable and may deteriorate the quality of the final product during the shelf-life.
(3) Lactose hydrolysis followed by severe heat treatment, such as in UHT applications, will result in Maillard reaction being propagated at an accelerated rate. When lactose is hydrolyzed to glucose and galactose, the concentration of sugars having reducing ends is doubled and especially galactose is much more reactive than lactose.

Recently and specifically in the case of lactose-free ESL and UHT milk drinks, advanced process engineering technologies in the form of aseptic dosing equipment have gained a lot of interest and made it possible to overcome many of the concerns linked to the batch process. Such aseptic dosing equipment makes it possible to add the lactase after the ESL/UHT treatment. Examples of such aseptic dosing equipment are Tetra Pak Aldose system, Tetra Pak Flexdose System, and GEA Varidose system. The advantages of these systems are:
(1) Dosing smaller quantities of sterile lactases with high precision into the milk stream after the heat treatment step, allowing lactose hydrolysis to take place during the first few days of storage.
(2) Decreasing the extent of Maillard reaction, browning and the formation of Advanced Glycation End products (AGEs) in lactose free milk drinks, especially when proper controlled storage conditions are applied.
(3) Omitting the pre-incubation compared to the batch process and thus solving the capacity issues and decreasing the risk of the activity of psychotropic microbes.

Despite the above advantages of aseptic dosing systems, there are a number of drawbacks associated with their use, such as:
(1) Since the lactases are added after the heat treatment step, their formulation should be of the highest possible purity. This is because any detrimental side activity in their formulation may have very negative impact on the end product during the long shelf-life especially in the case of UHT products.
(2) In the case of Tetra Pak Flexdose and GEA Varidose systems, the lactase enzyme should be aseptically filled in sterile buckets and bags.
(3) The above requirements in points (1) and (2) mean these sterile lactases cost more per unit of their activity compared to lactases used in the batch process.
(4) The average capital cost of these aseptic dosing systems is substantial.
(5) There are additional running costs linked to the consumables when using Tetra Pak Flexdose and GEA Varidose systems. Examples are changing the hose and needle every time an aseptic bucket is changed.
(6) In Tetra Pak Aldose system, the enzyme formulation is not sterile initially. It is diluted with water and then filtered in line (in the dairy) using at least 2 filters to ensure sterility of the enzyme stream before mixing it with the milk stream. Therefore, it can be problematic as the filters installed in-line may get blocked by poorly filterable enzyme formulation, which may cause a lot of trouble in operation. Even when the filterability of the enzyme formulation works as expected, there is still a need to change the filters regularly (usually daily).

A quick, smooth, easy to implement, trouble-free, cost-effective solution of applying lactases in lactose-free UHT and ESL products, which overcomes all the above limitations of both batch and aseptic dosing processes, does not exist yet.

WO2009/071539 (Novozymes) relates to a method of producing a dairy product using an enzyme having lactase activity. Disclosed is a method of producing a low-lactose milk product by treating a milk-based substrate with lactase at high temperature, i.e., at least 60° C., at least 62° C., at least 63° C., at least 64° C., at least 65° C., at least 67° C., at least 70° C., or at least 75° C.

WO2018/189238 (Chr. Hansen) discloses beta-galactosidases which are said to be stable with relatively high activity at a broad range of temperatures and pH values. Disclosed is a method for producing a dairy product by treating a milk-bases substrate with a beta-galactosidase, wherein the treatment or part of the treatment may take place at high temperature. A lactose concentration of less than 0.2% lactose may be obtained in 3-30 minutes after adding the beta-galactosidase.

WO2020/176734 (DuPont) relates to a method for reducing the amount of lactose in a milk-based substrate by contacting the substrate with a lactase, such as a thermostable lactase, at high temperature. Disclosed is a method for production of a lactose free dairy product from a milk-based substrate with an enzyme having neutral lactase activity wherein more than 20% lactase activity remains in the milk-based substrate after pasteurization at 72° C. for 15 seconds. Such pasteurization is also sometimes referred to as high-temperature, short-time (HTST) pasteurization.

US2010/0215828 relates to a process for preparing a well-preserving low-lactose, lactose-free or carbohydrate-free milk product, where sugars and proteins are separated into separate fractions and at least the protein fraction is thermally treated to inactivate the natural plasmin enzyme systems and other harmful enzymes, the protein and sugar fractions are heat-treated separately (to avoid Maillard reaction) and one or more of the fractions are combined into a milk product with a desired composition and sweetness. The heat treatment may be performed by pasteurizing, high pasteurizing, using ESL treatment or UHT treatment. If desired, the lactose in the sugar fraction may be hydrolyzed. Hydrolysis using lactase is followed by 4 hours incubation at 37° C. before the heat treatment.

US2013/0142904 (Arla) relates to a method of producing a packaged, lactose-reduced milk-related product, where a lactose-reduced milk-related feed is subjected to a High Temperature treatment and packaged.

Deeth (2017) “Optimum Thermal Processing for Extended Shelf-Life (ESL) Milk”, Foods 6(11): 102 has reviewed the optimum thermal processing for Extended Shelf-Life (ESL) milk. Deeth explains that ESL or ultra-pasteurized milk is produced by thermal processing using conditions between those used for traditional high-temperature, short-time (HTST) pasteurization and those used for ultra-high-temperature (UHT) sterilization. ESL milk should have a refrigerated shelf-life of more than 30 days. To achieve this, the thermal processing has to be quite intense. Unlike the temperature-time conditions for pasteurization, which are specified in most countries to be at least 72° C. C for at least 15 s, there are generally no such specified conditions for ESL processing. According to Deeth (2017), reported commercial processing conditions for ESL milk are mostly in the range 123-127° C. for 1-5 seconds. U.S. regulations define the process of “ultra-pasteurization” as heating milk at at least 138° C. for at least 2 seconds.

UHT treatment may be, e.g., heat treatment for 30 seconds at 130° C., for 3-4 seconds at 140° C. or for 1 second at 145° C.

### SUMMARY OF THE INVENTION

The invention provides a method of producing lactose-reduced heat-treated milk-based product, e.g. milk, using an enzyme having lactase activity without the need to perform an extensive preincubation of the milk-based substrate with the enzyme and without the need to use aseptic dosing systems to add the enzyme after the heat treatment.

The invention provides a method of producing a lactose-reduced heat-treated milk-based product which comprises:
a) adding an enzyme having lactase activity to a milk-based substrate comprising at least 2% lactose (w/w),
b) after addition of the enzyme, performing a heat treatment of the milk-based substrate by holding said milk-based substrate at a holding temperature of at least 120° C. for a holding time of at least 1 second followed by cooling to produce a heat-treated milk-based product, and
c) storing the heat-treated milk-based product for at least 24 hours, preferably at least 2 days, such as at least 3 days, more preferably at least 4 days, at a temperature of at most 40° C.,
   wherein after step c) the lactose content in the milk-based product is at most 0.2% (w/w).

Preferably, after step b) but before step c) the lactose content in the milk-based product is at least 0.5% (w/w).

Preferably, the enzyme having lactase activity is added immediately before the heat-treatment, such as the UHT treatment. After the heat-treatment, such as the UHT treatment, the enzyme has some residual activity which ensures lactose degradation to the desired low lactose level during storage in the cold or at ambient temperature. Preferably, the enzyme has a temperature optimum of 30-60° C., more preferably 35-55° C. Without wishing to be bound by theory, an enzyme having a higher temperature optimum and perhaps even being active during the heat treatment will have a somewhat rigid structure and will not have sufficient activity during storage in the cold or at ambient temperature. Again without wishing to be bound by theory, an enzyme having a temperature optimum of 30-60° C., more preferably 35-55° C., to be used in the method of the invention may unfold and be inactive during the heat treatment but have the ability to refold and be reactivated afterwards and therefore have a measurable or even substantial residual activity ensuring lactose degradation during storage of the milk-based product. It is contemplated that the rather high residual activity of the lactase enzymes of the invention after heat treatment such as UHT treatment may be due to an ability to refold and become reactivated once the temperature is lowered.

Preferably, the enzyme has a residual activity of at least 0.5%, preferably at least 1%, at least 2% or at least 3%, more preferably at least 5%, even more preferably at least 10%, after incubation in skimmed milk having a lactose content of 4.7% at 90° C. for 30 seconds, at 140° C. for 5 seconds and at 70° C. for 30 seconds followed by cooling to 0-10° C. and subsequent incubation at 23° C. for 72 hours, wherein the residual activity is relative to the activity of the same enzyme in skimmed milk without incubation at 90° C. for 30 seconds, at 140° C. for 5 seconds and at 70° C. for 30 seconds followed by cooling to 0-10° C. and subsequent incubation at 23° C. for 72 hours.

Preferably, the milk-based substrate is not incubated with the lactase before the heat treatment except for the time it takes—depending on the process equipment—from addition of the lactase to the milk-based substrate has reached the holding temperature of the heat treatment. Preferably, step b) is performed immediately after step a) without a dedicated incubation step.

At least three main process options can be applied according to the method:

In a first option, the milk-based substrate may be mixed with the lactase, and then processed directly under UHT or ESL conditions without the need to incubate the milk-based substrate with the lactase.

In a second option, the milk-based substrate may be processed directly under UHT or ESL conditions where the lactase is added to the milk-based substrate while said milk-based substrate is streaming through process pipes immediately before the heat treatment step, optionally while the temperature of the milk-based substrate is increasing towards the temperature of the heat treatment step. The lactase may be added at any point where the temperature of the milk-based substrate is 1-95° C., preferably 70-90° C., just before the UHT/ESL treatment. The addition of the lactase may take place via a simple dosing pump through a tube which is connected to the main milk stream tube. Once added to the running stream of milk-based substrate, the temperature is (further) raised to the ESL or UHT processing conditions. A heating medium may be used which is not in direct contact with the milk-based substrate but separated by equipment contact surfaces, such as a plate heat-exchanger or a tubular heat-exchanger. This may be referred to as an indirect heat treatment, preferably an indirect UHT treatment.

In a third option, the heat treatment is performed by steam injection or steam infusion, preferably steam injection, using high-pressure steam to heat the milk-based substrate, and the enzyme may be added with the steam. This may be referred to as a direct heat treatment, preferably a direct UHT treatment. After the holding time of step b), the milk-based substrate comprising the steam may be flash-cooled in a vacuum to remove water equivalent to amount of condensed steam used. In addition to the heating of the milk-based substrate by steam injection or steam infusion, indirect heating may also be applied, e.g., using a plate or tubular heat exchanger.

In any case, the residual activity of the lactase after the heat treatment ensures that lactose levels will decrease to lactose-reduced, preferably lactose-free levels (e.g., less than 0.1% or less than 0.01%) during the initial stage of storage (e.g., up to 2 weeks, such as during the first 2 or 3 days).

The method of the invention has a number of advantages over the processes used today for production of ESL- and UHT-treated milk-based products, such as ESL or UHT milk.

Compared to the batch processes applied today, the process of the invention provides an improved colour and quality of the lactose-reduced or lactose free milk-based product due to reduced Maillard reaction. Without preincubation, there will be reduced growth of psychotropic bacteria secreting enzymes including proteases possibly surviving the heat treatment, and therefore the milk-based product can have a longer shelf life. Further, without preincubation, capacity cost and process time is reduced. The method of the invention is also easier to operate since there is no need to monitor the tanks until the lactose level is below 0.1% or 0.01%.

Compared to the aseptic dosing processes applied today, in the process of the invention there is no need to invest in the aseptic dosing equipment and no need for, e.g., regular changing of the aseptic bucket. Further, the current sterile lactases have approximately 1-year-shelflife, while the enzyme used in the method of the invention can have a 2-year shelf life or longer. The method of the invention is easy to operate and hassle-free as there is no need to monitor or troubleshoot any of the aseptic dosing systems. The final quality of the milk-based product is the same regarding colour and Maillard reaction.

The method of the invention is easy to apply on an industrial level and there is no barrier for its implementation.

The inventors have surprisingly found that lactase enzymes from the CAZy database GH2 family clade DYLGE are particularly suitable to be used in the methods of the invention. Therefore, in preferred embodiments, the enzyme having lactase activity is a GH2 lactase from clade DYLGE, preferably a bacterial GH2 lactase from clade DYLGE.

In preferred embodiments, the enzyme having lactase activity comprises in its amino acid sequence the motif WTXXDY[I/L/R]GE[P/S/A].

In preferred embodiments, the enzyme having lactase activity comprises in its amino acid sequence the motif(s) SR[W/Y/F]YSGSGX[Y/G]R and/or [L/V/I]X[L/V/I]PHD.

### BRIEF DESCRIPTION OF DRAWINGS

FIG. 1 shows a dose-response curve for non-UHT treated B. bifidum lactase (SEQ ID NO: 1). B. bifidum lactase was added to skim milk in the following dosages: 2.03, 1.63, 1.30, 1.04, 0.832, 0.666, 0.532, 0.426, 0.341, 0.273, 0.218, 0.174, 0.140, 0.112, 0.0893, 0.0715 mg enzyme protein (ep) per litre skim milk and incubated for 3 days at 23° C. (without a UHT treatment). The residual lactose for each sample was determined as % (g lactose per 100 ml skim milk) and a dose/response curve made. The curve can be used to determine the relative activity of a UHT-treated lactase in % of the activity of non-UHT treated B. bifidum lactase. The lactase to be tested is added to skim milk at a dosage×mg lactase ep per litre skim milk, a UHT treatment is performed followed by incubation for 3 days at 23° C., and residual lactose is determined as % (g lactose per 100 ml skim milk). The dose/response curve is used to determine the “corresponding dosage” y (mg lactase ep per liter skim milk) of non-UHT treated B. bifidum lactase which would result in the same residual lactose after incubation in skim milk for 3 days at 23° C. The relative activity of the UHT-treated lactase in % of the activity of non-UHT treated B. bifidum lactase is calculated as y/x*100%.

### DEFINITIONS

In accordance with this detailed description, the following definitions apply. Note that the singular forms “a,” “an,” and “the” include plural references unless the context clearly dictates otherwise.

Unless defined otherwise or clearly indicated by context, all technical and scientific terms used herein have the same meaning as commonly understood by one of ordinary skill in the art to which this invention belongs.

GH2 lactase: The term “GH2 lactase”, “GH2 enzyme” or “GH2 polypeptide” in the context of the present invention means a lactase enzyme being classified as member of the Glycoside hydrolase family 2 in the database of Carbohydrate-Active EnZymes (CAZymes) (http://www.cazy.org/).

Isolated: The term “isolated” means a polypeptide, nucleic acid, cell, or other specified material or component that has been separated from at least one other material or component, including but not limited to, other proteins, nucleic acids, cells, etc. An isolated polypeptide, nucleic acid, cell or other material is thus in a form that does not occur in nature. An isolated polypeptide includes, but is not limited to, a culture broth containing the secreted polypeptide expressed in a host cell.

Lactase: The term “lactase” means a glycoside hydrolase having the ability to hydrolyse the disaccharide lactose into constituent galactose and glucose monomers. The group of lactases comprises but is not limited to enzymes assigned to subclass EC 3.2.1.23 and EC 3.2.1.108. Enzymes assigned to other subclasses, such as, e.g., EC 3.2.1.21, may also be lactases in the context of the present invention. A lactase in the context of the invention may have other activities than the lactose hydrolysing activity, such as for example a transgalactosylating activity. In the context of the invention, the lactose hydrolysing activity of the lactase may be referred to as its lactase activity, its beta-galactosidase activity or its hydrolysing activity.

Lactase Activity: Lactase activity may be determined using, e.g., a LAU(B) assay. The activity in LAU(B) of a specific lactase may be determined by direct measurement of o-nitrophenyl (ONP) released from o-nitrophenyl β-D-galactopyranoside (ONPG) in a buffer containing 1.46 mg/ml substrate in 0.05 M MES, 1 mM MgSO4 7H2O, 450 mg/L Brij 35 at pH6.5 and 30° C. After 600 seconds incubation, the reaction is stopped by adding 0.2 M Na2CO3 and the released ONP is measured at 405 nm after 126 seconds incubation. The activity is obtained by comparing to a standard curve run with a lactase of known activity, and the activity of the unknown sample calculated from this. The lactase of known activity may, e.g., be Saphera® obtained from Novozymes A/S, Denmark. Lactase activity may be determined by measuring the amount of lactose hydrolysis in milk, e.g. by the method described in Example 4 in the paragraph “Analysis of residual lactose content” using HPAEC-PAD where the lactose peak is related to a lactose standard with known concentration. The lactose hydrolysis can then be related to the amount of lactase added, e.g. per mg enzyme protein or per mole enzyme. Other methods for measuring lactase activity are known and used routinely in the art.

Mature polypeptide: The term “mature polypeptide” means a polypeptide in its mature form following N terminal and/or C-terminal processing (e.g., removal of signal peptide).

Milk: The term “milk” means the lacteal secretion obtained by milking any mammal, such as cows, sheep, goats, buffaloes or camels.

Milk-based product: The term “milk-based product” refers to milk products and other products based on milk. In the present context it will be apparent that the term includes in particular heat-treated milk-based products, including not only pasteurized milk but also milk that is subjected to higher temperatures than those typically used in pasteurization, such as ultra-pasteurized milk, UHT (ultra-high temperature) milk and ESL (extended shelf life) milk.

Purified: The term “purified” means a nucleic acid, polypeptide or cell that is substantially free from other components as determined by analytical techniques well known in the art (e.g., a purified polypeptide or nucleic acid may form a discrete band in an electrophoretic gel, chromatographic eluate, and/or a media subjected to density gradient centrifugation). A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight or on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

In one aspect, the term “purified” as used herein refers to the polypeptide or cell being essentially free from components (especially insoluble components) from the production organism. In other aspects. the term “purified” refers to the polypeptide being essentially free of insoluble components (especially insoluble components) from the native organism from which it is obtained. In one aspect, the polypeptide is separated from some of the soluble components of the organism and culture medium from which it is recovered. The polypeptide may be purified (i.e., separated) by one or more of the unit operations filtration, precipitation, or chromatography.

Accordingly, the polypeptide may be purified such that only minor amounts of other proteins, in particular, other polypeptides, are present. The term “purified” as used herein may refer to removal of other components, particularly other proteins and most particularly other enzymes present in the cell of origin of the polypeptide. The polypeptide may be “substantially pure”, i.e., free from other components from the organism in which it is produced, e.g., a host organism for recombinantly produced polypeptide. In one aspect, the polypeptide is at least 40% pure by weight of the total polypeptide material present in the preparation. In one aspect, the polypeptide is at least 50%, 60%, 70%, 80% or 90% pure by weight of the total polypeptide material present in the preparation. As used herein. a “substantially pure polypeptide” may denote a polypeptide preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polypeptide material with which the polypeptide is natively or recombinantly associated.

It is, therefore, preferred that the substantially pure polypeptide is at least 92% pure, preferably at least 94% pure, more preferably at least 95% pure, more preferably at least 96% pure, more preferably at least 97% pure, more preferably at least 98% pure, even more preferably at least 99% pure, most preferably at least 99.5% pure by weight of the total polypeptide material present in the preparation. The polypeptide of the present invention is preferably in a substantially pure form (i.e., the preparation is essentially free of other polypeptide material with which it is natively or recombinantly associated). This can be accomplished, for example by preparing the polypeptide by well-known recombinant methods or by classical purification methods.

Sequence identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the nobrief option must be specified in the command line. The output of Needle labeled “longest identity” is calculated as follows:

(Identical Residues×100)/(Length of Alignment−Total Number of Gaps in Alignment)

Variant: The term “variant” means a polypeptide having lactase activity comprising a man-made mutation, i.e., a substitution, insertion (including extension), and/or deletion (e.g., truncation), at one or more positions. A substitution means replacement of the amino acid occupying a position with a different amino acid; a deletion means removal of the amino acid occupying a position; and an insertion means adding 1-5 amino acids (e.g., 1-3 amino acids, in particular 1 amino acid) adjacent to and immediately following the amino acid occupying a position.

Wild-type: The term “wild-type” in reference to an amino acid sequence or nucleic acid sequence means that the amino acid sequence or nucleic acid sequence is a native or naturally-occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).

### Conventions for Designation of Variants:

For purposes of the present invention, a polypeptide having a chosen wild-type sequence may be used to determine the corresponding amino acid positions in another lactase. The amino acid sequence of another lactase is aligned with the polypeptide having a chosen wild-type sequence, and based on the alignment, the amino acid position number corresponding to any amino acid residue in the polypeptide having a chosen wild-type sequence is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

In describing the variants of the present invention, the nomenclature described below is adapted for ease of reference. The accepted IUPAC single letter or three letter amino acid abbreviation is employed. The amino acid numbering of the variants disclosed herein is in each case based on the numbering of the relevant wild-type sequence.

For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”) or simply by a space, e.g., substitutions at positions 205 and 411 of glycine (G) with arginine (R) and serine(S) with phenylalanine (F) may be represented by “Gly205Arg+Ser411Phe”, “G205R+S411F”, “Gly205Arg Ser411Phe” or “G205R S411F”.

### DETAILED DESCRIPTION OF THE INVENTION

The invention provides a method of producing a lactose-reduced heat-treated milk-based product which comprises:
a) adding an enzyme having lactase activity to a milk-based substrate comprising at least 2% lactose (w/w),
b) after addition of the enzyme, performing a heat treatment of the milk-based substrate by holding said milk-based substrate at a holding temperature of at least 120° C. for a holding time of at least 1 second followed by cooling to produce a heat-treated milk-based product, and
c) storing the heat-treated milk-based product for at least 24 hours, preferably at least 2 days, such as at least 3 days, more preferably at least 4 days, at a temperature of at most 40° C.,
   wherein after step c) the lactose content in the milk-based product is at most 0.2% (w/w).

Preferably, after step b) but before step c), the lactose content in the milk-based product is at least 0.5% (w/w).

The milk-based substrate preferably comprises 2-30%, preferably 2-17% (w/w), more preferably 4-5.5 (w/w), lactose.

The milk-based substrate may be any raw and/or processed milk material. Useful milk-based substrates include, but are not limited to, solutions/suspensions of any milk or milk like products comprising lactose, such as whole or low-fat milk, skim milk, buttermilk, flavored milk such as chocolate milk, reconstituted milk powder, condensed milk, solutions of dried milk, milk comprising skim milk powder, milk permeate, whey, whey permeate, acid whey, or cream.

The milk-based substrate may be milk, such as raw milk, e.g., raw milk which has not been pasteurized before step a).

In a preferred embodiment, the milk-based substrate is milk, condensed milk or milk comprising skim milk powder.

In a more preferred embodiment, the milk-based substrate is milk comprising 4-5.5%, preferably 4.5-5%, lactose (w/w).

In one embodiment, the milk-based substrate is raw milk, preferably raw milk which has not been pasteurized before step a.

In one embodiment, after step a) but before step b), the milk-based substrate is incubated for at most 4 hours, preferably at most 60 minutes, more preferably at most 10 minutes, even more preferably at most 5 minutes. Such incubation may be performed at a temperature of at most 10° C., preferably at most 7° C.

However, preferably, step b) is performed immediately after step a) without a dedicated incubation step.

Preferably, the time from addition of the enzyme until the holding temperature of step b) is reached is at most 5 minutes, more preferably at most 2 minutes, even more preferably at most 1 minute.

In a preferred embodiment, the heat treatment is performed as an indirect heat treatment, preferably an indirect UHT treatment. Pumping equipment may have been fitted to add the enzyme to the milk-based substrate while said milk-based substrate is streaming through process equipment, such as process pipes. A heating medium may be used, such as a plate heat-exchanger or a tubular heat-exchanger, which is not in direct contact with the milk-based substrate but separated by equipment contact surfaces. Preferably, the enzyme is added to the milk-based substrate immediately before the heat treatment step, such as immediately before the holding temperature is reached, optionally while the temperature of the milk-based substrate is increasing towards the holding temperature of step b).

In another preferred embodiment, the heat treatment is performed as a direct heat treatment, preferably a direct UHT treatment. The heat treatment may be performed by steam injection or steam infusion, preferably steam injection, using high-pressure steam to heat the milk-based substrate. In a preferred embodiment, the enzyme is added with the steam. Preferably, after the holding time of step b), the milk-based substrate comprising the steam is flash-cooled in a vacuum to remove water equivalent to amount of condensed steam used.

A combination of direct and indirect heat treatment may also be used.

The heat treatment may be an ESL treatment, an ultra-pasteurization or a UHT treatment.

The heat treatment may be performed at a temperature of 120-150° C.

In one embodiment, the heat treatment is performed at a temperature of at least 123° C., preferably at 123-145° C.

In one embodiment, the heat treatment is performed at a temperature of at least 130° C., preferably at 130-145° C.

In one embodiment, the heat treatment is performed at a temperature of at least 138° C., preferably at 138-145° C., more preferably at 138-142° C.

The holding time of step b) may be 1-30 seconds, preferably 1-10 seconds, more preferably 1-5 seconds.

In one embodiment, the heat treatment is a UHT treatment, preferably a UHT treatment performed at a temperature of 130-145° C. for a time of 1-30 seconds, more preferably at a temperature of 138-145° C. for a time of 1-10 seconds, even more preferably at a temperature of 138-144° C. for a time of 1-5 seconds.

In one embodiment, the heat treatment is a UHT treatment performed at a temperature of 128-132° C. for 25-35 seconds, at a temperature of 138-140° C. for 2-5 seconds or at a temperature of 144-146° C. for 0.5-2 seconds.

In one embodiment, the heat treatment is an ESL treatment or an ultra-pasteurization, preferably an ESL treatment or an ultra-pasteurization performed at a temperature of 120-140° C. for a time of 1-5 seconds, more preferably an ESL treatment or an ultra-pasteurization performed at a temperature of 120-130° C. for 1-5 seconds or at a temperature of 138-140° C. for 2-4 seconds.

Preferably, no enzyme having lactase activity is added to the milk-based product after step b), such as after the holding time of step b). More preferably, no enzyme is added to the milk-based product after step b), such as after the holding time of step b). Even more preferably, nothing is added to the milk-based product after step b), such as after the holding time of step b). This is because, after the heat-treatment, the milk-based product is sterile, and adding anything, even something which is considered sterile, will be at the risk of contaminating the product.

After the holding time of step b), the milk-based product is cooled to at most 40° C., preferably at most 35° C., more preferably at most 30° C., preferably within 5 minutes, more preferably within 3 minutes, even more preferably within 2 minutes, such as within 1 minute.

Preferably, the time from addition of the enzyme until the heat-treated milk-based product has been cooled to a temperature of at most 40° C., preferably at most 35° C., more preferably at most 30° C., is at most 3.5 minutes, preferably at most 3 minutes, more preferably at most 2.5 minutes, such as at most 2 minutes or at most 1 minute.

After step b) but before step c) the milk-based product may be homogenized.

Alternatively, homogenization may be performed before reaching the holding temperature of step b). In indirect UHT treatments (e.g., tube exchange or plate exchange), homogenization is preferably performed upstream. In direct UHT treatments (e.g., steam injection or steam infusion), homogenization is preferably performed downstream.

Preferably, after step b) but before step c) the milk-based product is aseptically packed.

In a preferred embodiment, the milk-based product is UHT milk. UHT milk in the context of the present invention is milk which has been subjected to a sterilization procedure which is intended to kill all microorganisms, including the bacterial spores.

Preferably, less than 80% of the lactose has been hydrolyzed when step b) is completed, and more than 90% of the lactose has been hydrolyzed after one week. More preferably, less than 60% of the lactose has been hydrolyzed when step b) is completed, and more than 95% of the lactose has been hydrolyzed after one week.

Preferably, after step b), the enzyme retains at least 0.01% of its initial activity, more preferably at least 0.1%, more preferably at least 1%, more preferably at least 2%, more preferably at least 10%, more preferably at least 50%, even more preferably at least 80%, most preferably at least 90%. The enzyme activity retained after step b) in percent of the enzyme activity when the enzyme is added may be determined, e.g., by using a “Residual activity assay” of the Examples.

Preferably, step c) is performed at a temperature of 2-40° C., preferably 15-40° C., more preferably 18-40° C., most preferably 18-30° C. In a preferred embodiment, step c) is performed at room temperature which may vary during the storage period.

In a preferred embodiment, after step b) but before step c) the lactose content in the milk-based product is at least 1% (w/w), such as at least 2% (w/w), at least 3% (w/w) or at least 4% (w/w).

In another preferred embodiment, after step b) but before step c) the lactose content in the milk-based product has been reduced by at most 80%, preferably at most 50%, more preferably at most 20%, compared to the lactose content before step a).

In step c), the heat-treated milk-based product is stored for at least 24 hours, preferably at least 2 days, such as at least 3 days, more preferably at least 4 days, such as at least 7 days, even more preferably at least 14 days, such as at least 21 days. It is primarily during this storage period that the lactose level is decreased to the desired level by the residual activity of the lactase.

In a preferred embodiment, after having stored the heat-treated milk-based product for 21 days at a temperature of 2-40° C., preferably 15-40° C., more preferably 18-40° C., most preferably 18-30° C., the lactose content in the milk-based product is at most 0.2% (w/w), preferably at most 0.1%, more preferably at most 0.01%.

In another preferred embodiment, after having stored the heat-treated milk-based product for 14 days at a temperature of 2-40° C., preferably 15-40° C., more preferably 18-40° C., most preferably 18-30° C., the lactose content in the milk-based product is at most 0.2% (w/w), preferably at most 0.1%, more preferably at most 0.01%.

In another preferred embodiment, after having stored the heat-treated milk-based product for 7 days at a temperature of 2-40° C., preferably 15-40° C., more preferably 18-40° C., most preferably 18-30° C., the lactose content in the milk-based product is at most 0.2% (w/w), preferably at most 0.1%, more preferably at most 0.01%.

In another preferred embodiment, after having stored the heat-treated milk-based product for 4 days at a temperature of 2-40° C., preferably 15-40° C., more preferably 18-40° C., most preferably 18-30° C., the lactose content in the milk-based product is at most 0.2% (w/w), preferably at most 0.1%, more preferably at most 0.01%.

In another preferred embodiment, after having stored the heat-treated milk-based product for 3 days at a temperature of 2-40° C., preferably 15-40° C., more preferably 18-40° C., most preferably 18-30° C., the lactose content in the milk-based product is at most 0.2% (w/w), preferably at most 0.1%, more preferably at most 0.01%.

In another preferred embodiment, after having stored the heat-treated milk-based product for 2 days at a temperature of 2-40° C., preferably 15-40° C., more preferably 18-40° C., most preferably 18-30° C., the lactose content in the milk-based product is at most 0.2% (w/w), preferably at most 0.1%, more preferably at most 0.01%.

In another preferred embodiment, after having stored the heat-treated milk-based product for 24 hours at a temperature of 2-40° C., preferably 15-40° C., more preferably 18-40° C., most preferably 18-30° C., the lactose content in the milk-based product is at most 0.2% (w/w), preferably at most 0.1%, more preferably at most 0.01%.

In a preferred embodiment, after having stored the heat-treated milk-based product for 21 days at a temperature of 2-40° C., preferably 15-40° C., more preferably 18-40° C., most preferably 18-30° C., the lactose content in the milk-based product has been reduced by at least 80% or at least 85%, preferably at least 90, 91, 92, 93, 94, 95, 96 or 97%, more preferably at least 98%, even more preferably at least 99% or at least 99.5%, and most preferably at least 99.8% or at least 99.9%, compared to the lactose content before step a).

In another preferred embodiment, after having stored the heat-treated milk-based product for 14 days at a temperature of 2-40° C., preferably 15-40° C., more preferably 18-40° C., most preferably 18-30° C., the lactose content in the milk-based product has been reduced by at least 80% or at least 85%, preferably at least 90, 91, 92, 93, 94, 95, 96 or 97%, more preferably at least 98%, even more preferably at least 99% or at least 99.5%, and most preferably at least 99.8% or at least 99.9%, compared to the lactose content before step a).

In another preferred embodiment, after having stored the heat-treated milk-based product for 7 days at a temperature of 2-40° C., preferably 15-40° C., more preferably 18-40° C., most preferably 18-30° C., the lactose content in the milk-based product has been reduced by at least 80% or at least 85%, preferably at least 90, 91, 92, 93, 94, 95, 96 or 97%, more preferably at least 98%, even more preferably at least 99% or at least 99.5%, and most preferably at least 99.8% or at least 99.9%, compared to the lactose content before step a).

In another preferred embodiment, after having stored the heat-treated milk-based product for 4 days at a temperature of 2-40° C., preferably 15-40° C., more preferably 18-40° C., most preferably 18-30° C., the lactose content in the milk-based product has been reduced by at least 80% or at least 85%, preferably at least 90, 91, 92, 93, 94, 95, 96 or 97%, more preferably at least 98%, even more preferably at least 99% or at least 99.5%, and most preferably at least 99.8% or at least 99.9%, compared to the lactose content before step a).

In another preferred embodiment, after having stored the heat-treated milk-based product for 3 days at a temperature of 2-40° C., preferably 15-40° C., more preferably 18-40° C., most preferably 18-30° C., the lactose content in the milk-based product has been reduced by at least 80% or at least 85%, preferably at least 90, 91, 92, 93, 94, 95, 96 or 97%, more preferably at least 98%, even more preferably at least 99% or at least 99.5%, and most preferably at least 99.8% or at least 99.9%, compared to the lactose content before step a).

In another preferred embodiment, after having stored the heat-treated milk-based product for 2 days at a temperature of 2-40° C., preferably 15-40° C., more preferably 18-40° C., most preferably 18-30° C., the lactose content in the milk-based product has been reduced by at least 80% or at least 85%, preferably at least 90, 91, 92, 93, 94, 95, 96 or 97%, more preferably at least 98%, even more preferably at least 99% or at least 99.5%, and most preferably at least 99.8% or at least 99.9%, compared to the lactose content before step a).

In another preferred embodiment, after having stored the heat-treated milk-based product for 24 hours at a temperature of 2-40° C., preferably 15-40° C., more preferably 18-40° C., most preferably 18-30° C., the lactose content in the milk-based product has been reduced by at least 80% or at least 85%, preferably at least 90, 91, 92, 93, 94, 95, 96 or 97%, more preferably at least 98%, even more preferably at least 99% or at least 99.5%, and most preferably at least 99.8% or at least 99.9%, compared to the lactose content before step a).

In a preferred embodiment, after having stored the heat-treated milk-based product for 21 days at a temperature of 2-40° C., preferably 15-40° C., more preferably 18-40° C., most preferably 18-30° C., the lactose content in the milk-based product is at most 1000 ppm, preferably at most 100 ppm.

In another preferred embodiment, after having stored the heat-treated milk-based product for 14 days at a temperature of 2-40° C., preferably 15-40° C., more preferably 18-40° C., most preferably 18-30° C., the lactose content in the milk-based product is at most 1000 ppm, preferably at most 100 ppm.

In another preferred embodiment, after having stored the heat-treated milk-based product for 7 days at a temperature of 2-40° C., preferably 15-40° C., more preferably 18-40° C., most preferably 18-30° C., the lactose content in the milk-based product is at most 1000 ppm, preferably at most 100 ppm.

In another preferred embodiment, after having stored the heat-treated milk-based product for 4 days at a temperature of 2-40° C., preferably 15-40° C., more preferably 18-40° C., most preferably 18-30° C., the lactose content in the milk-based product is at most 1000 ppm, preferably at most 100 ppm.

In another preferred embodiment, after having stored the heat-treated milk-based product for 3 days at a temperature of 2-40° C., preferably 15-40° C., more preferably 18-40° C., most preferably 18-30° C., the lactose content in the milk-based product is at most 1000 ppm, preferably at most 100 ppm.

In another preferred embodiment, after having stored the heat-treated milk-based product for 2 days at a temperature of 2-40° C., preferably 15-40° C., more preferably 18-40° C., most preferably 18-30° C., the lactose content in the milk-based product is at most 1000 ppm, preferably at most 100 ppm.

In another preferred embodiment, after having stored the heat-treated milk-based product for 24 hours at a temperature of 2-40° C., preferably 15-40° C., more preferably 18-40° C., most preferably 18-30° C., the lactose content in the milk-based product is at most 1000 ppm, preferably at most 100 ppm.

After step c), the lactose-reduced heat-treated milk product may—if desired—be freeze-dried.

The enzyme having lactase activity may be added at a concentration of 100-50,000, preferably 500-40,000 LAU(B) per liter milk-based substrate.

The enzyme having lactase activity may be added at a concentration of 1-150 mg enzyme protein per litre milk-based substrate, preferably 1-100, more preferably 2-50 or 5-50 mg enzyme protein per litre milk-based substrate.

Before step b), a reducing agent may be added to the milk-based substrate, preferably a food-approved reducing agent, more preferably a reducing agent selected among L-cysteine, sulphite and glutathione. The reducing agent nay be added together with the enzyme, f.ex., the reducing agent may be part of the enzyme formulation. The reducing agent may be added to lower or prevent oxidation of, e.g., cysteines in the lactase enzyme which do not form part of a disulfide bridge, sometimes referred to as “free cysteines”. Oxidation of such free cysteines may lower the ability of the enzyme to refold after the thermal treatment, such as the UHT treatment.

The lactase enzyme of SEQ ID NO: 1 has a free cysteine residue at position C372, the oxidation of which has been shown to lower the ability of the enzyme to refold, and a corresponding cysteine residue can be identified in many other GH2 lactase enzymes of clade DYLGE by aligning their amino acid sequence with SEQ ID NO: 1. In a preferred embodiment, the enzyme having lactase activity has an amino acid substitution of the cysteine corresponding to C372 of SEQ ID NO: 1, preferably to serine, alanine or glycine.

Preferably, the enzyme having lactase activity is a lactase. Preferably, the enzyme belongs to enzyme class 3.2.1.21, 3.2.1.23 or 3.2.1.108, more preferably 3.2.1.23 or 3.2.1.108.

In a preferred embodiment, the enzyme having lactase activity is a neutral lactase.

In a preferred embodiment, the enzyme having lactase activity is purified.

In a preferred embodiment, the enzyme having lactase activity is isolated.

Preferably, the enzyme having lactase activity is a bacterial enzyme.

Preferably, the enzyme having lactase activity is a GH2 lactase, more preferably a GH2 lactase of clade DYLGE. The Carbohydrate-Active enZYmes Database (CAZy) which has been online since 1998 (http://www.cazy.org/) is a specialist database which classifies carbohydrate-active enzymes (CAZymes) into families (Lombard V, Golaconda Ramulu H, Drula E, Coutinho P M, Henrissat B (2014) The Carbohydrate-active enzymes database (CAZy), 2013, Nucleic Acids Res 42:D490-D495; Elodie Drula, Marie-Line Garron, Suzan Dogan, Vincent Lombard, Bernard Henrissat, Nicolas Terrapon, The carbohydrate-active enzyme database: functions and literature, Nucleic Acids Research, Volume 50, Issue D1, 7 Jan. 2022, Pages D571-D577).

The Glycoside Hydrolase (GH) family consist of enzymes which catalyze hydrolysis and/or rearrangement of glycosidic bonds, and these are further classified into (sub-) families, such as GH2, which again can be subdivided into clades based on sequence motifs. Lactases classified as GH2 of clade DYLGE have by the present inventors been found to be particularly good at retaining activity after a thermal treatment and are therefore particularly suitable in the methods of the invention.

GH2 lactases of the DYLGE clade comprise the motif WTXXDY[I/L/R]GE[P/S/A]. The glutamic acid E in the motif is involved in binding of galactose. Lactases of the DYLGE clade may also contain additional short peptide motifs SR[W/Y/F]YSGSGX[Y/G]R and/or [W/V/I]X[L/V/I]PHD, which are both located in the GH2 N-terminal galactose binding domain and are important for substrate binding.

In preferred embodiments, the enzyme having lactase activity comprises in its amino acid sequence the motif WTXXDY[I/L/R]GE[P/S/A].

In preferred embodiments, the enzyme having lactase activity comprises in its amino acid sequence the motif(s) SR[W/Y/F]YSGSGX[Y/G]R and/or [L/V/I]X[L/V/I]PHD.

Preferably, the enzyme having lactase activity has a temperature optimum of 30-60° C., preferably 35-55° C. The temperature optimum may be determined using Method 2 of the Examples.

Preferably, the enzyme having lactase activity has a melting temperature Tm of 50-70° C. determined by thermal shift at pH 6. In another preferred embodiment, the enzyme having lactase activity has a melting temperature Tm of 50-70° C. determined by thermal shift at pH 7. The melting temperature Tm may be determined using the thermal shift assay of Example 5.

Preferably, the enzyme having lactase activity has a residual activity of at least 1%, preferably at least 2%, more preferably at least 5%, even more preferably at least 10%, after incubation at 70° C. for 30 seconds and 140° C. for 5 seconds in skimmed milk having a lactose content of 4.7%. The residual activity may be determined as in Example 3.

In a preferred embodiment, the enzyme having lactase activity has an amino acid sequence which is at least 50%, such as at least 60%, at least 70%, at least 80%, at least 90%, at least 95%, at least 98%, at least 99%, at least 99.5% or 100% identical to any of SEQ ID NOs: 1, 2, 4, 5, 6, 7, 8, 9, 10, 11, 12, 13, 14 or 15 or to a mature polypeptide of any of these.

In another preferred embodiment, the enzyme having lactase activity has an amino acid sequence which is at least 50%, such as at least 60%, at least 70%, at least 80%, at least 90%, at least 95%, at least 98%, at least 99%, at least 99.5% or 100% identical to SEQ ID NO: 1.

In another preferred embodiment, the enzyme having lactase activity has an amino acid sequence which is at least 50%, such as at least 60%, at least 70%, at least 80%, at least 90%, at least 95%, at least 98%, at least 99%, at least 99.5% or 100% identical to SEQ ID NO: 4.

In another preferred embodiment, the enzyme having lactase activity has an amino acid sequence which is at least 50%, such as at least 60%, at least 70%, at least 80%, at least 90%, at least 95%, at least 98%, at least 99%, at least 99.5% or 100% identical to SEQ ID NO: 5.

In another preferred embodiment, the enzyme having lactase activity has an amino acid sequence which is at least 50%, such as at least 60%, at least 70%, at least 80%, at least 90%, at least 95%, at least 98%, at least 99%, at least 99.5% or 100% identical to SEQ ID NO: 6.

In another preferred embodiment, the enzyme having lactase activity has an amino acid sequence which is at least 50%, such as at least 60%, at least 70%, at least 80%, at least 90%, at least 95%, at least 98%, at least 99%, at least 99.5% or 100% identical to SEQ ID NO: 11.

In a preferred embodiment, the enzyme having lactase activity is a polypeptide derived from any of SEQ ID NOs: 1, 2, 4, 5, 6, 7, 8, 9, 10, 11, 12, 13, 14 or 15 or from a mature polypeptide of any of these having 1-30 alterations, e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions.

In another preferred embodiment, the enzyme having lactase activity is a polypeptide derived from SEQ ID NO: 1 having 1-30 alterations, e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions.

In another preferred embodiment, the enzyme having lactase activity is a polypeptide derived from SEQ ID NO: 4 having 1-30 alterations, e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions.

In another preferred embodiment, the enzyme having lactase activity is a polypeptide derived from SEQ ID NO: 5 having 1-30 alterations, e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions.

In another preferred embodiment, the enzyme having lactase activity is a polypeptide derived from SEQ ID NO: 6 having 1-30 alterations, e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions.

In another preferred embodiment, the enzyme having lactase activity is a polypeptide derived from SEQ ID NO: 11 having 1-30 alterations, e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions.

In a preferred embodiment, the enzyme having lactase activity is derived from any of SEQ ID NOs: 1, 2, 4, 5, 6, 7, 8, 9, 10, 11, 12, 13, 14 or 15 by substitution, deletion or addition of one or several amino acids.

In another preferred embodiment, the enzyme having lactase activity is derived from a mature polypeptide of any of SEQ ID NOs: 1, 2, 4, 5, 6, 7, 8, 9, 10, 11, 12, 13, 14 or 15 by substitution, deletion or addition of one or several amino acids.

In one embodiment, the number of amino acid substitutions, deletions and/or insertions introduced into the polypeptide of any of SEQ ID NOs: 1, 2, 4, 5, 6, 7, 8, 9, 10, 11, 12, 13, 14 or 15 is up to 15, e.g., 1, 2, 3, 4, 5, 6, 7, 8, 9, 10, 11, 12, 13, 14, or 15.

The amino acid changes may be of a minor nature, that is conservative amino acid substitutions or insertions that do not significantly affect the folding and/or activity of the protein; small deletions, typically of 1-30 amino acids; small amino or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are tested for lactase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide, and/or be inferred from sequence homology and conserved catalytic machinery with a related polypeptide or within a polypeptide or protein family with polypeptides/proteins descending from a common ancestor, typically having similar three-dimensional structures, functions, and significant sequence similarity. Additionally or alternatively, protein structure prediction tools can be used for protein structure modelling to identify essential amino acids and/or active sites of polypeptides. See, for example, Jumper et al., 2021, “Highly accurate protein structure prediction with AlphaFold”, Nature 596: 583-589.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Amino acid alterations may also be non-conservative alterations, for example non-conservative substitutions, deletions and/or insertions, typically substitutions, which provide the enzyme with one or more desired characteristics. Non-limiting examples of such desired characteristics include improved residual activity after being subjected to elevated temperature, refolding ability after denaturation, and specific activity.

In a preferred embodiment, the enzyme having lactase activity is derived from any of SEQ ID NOs: 1, 2, 4, 5, 6, 7, 8, 9, 10, 11, 12, 13, 14 or 15, preferably from SEQ ID NO: 1, 4, 5, 6 or 11, by substitution of 1-30, preferably 1-20, amino acids, so that the enzyme has a higher residual activity after a UHT treatment compared to the enzyme from which it is derived.

The enzyme having lactase activity may be obtained from microorganisms of any genus. For purposes of the present invention, the term “obtained from” as used herein in connection with a given source shall mean that the polypeptide encoded by a polynucleotide is produced by the source or by a strain in which the polynucleotide of the invention has been inserted. In one aspect, the polypeptide obtained from a given source is secreted extracellularly.

In a preferred embodiment, the enzyme having lactase activity is obtained from Bifidobacterium, preferably from Bifidobacterium bifidum, Bifidobacterium samirii, Bifidobacterium aerophilum or Bifidobacterium mongoliense, or from Bacillus, preferably from Bacillus circulans or Bacillus sp. S3, from Varibaculum sp., from Urmitella, preferably from Urmitella timonensis, from Streptococcus, preferably from Streptococcus entericus DSM 14446, from Streptomyces, preferably from Streptomyces cirratus, from Clostridium, preferably from Clostridium nexile CAG:348, or from Neobacillus, preferably from Neobacillus bataviensis, Neobacillus mesonae or Neobacillus niacini, or is a variant of an enzyme obtained from any of these genera or species.

In another preferred embodiment, the enzyme having lactase activity is obtained from Bifidobacterium, preferably from Bifidobacterium bifidum or Bifidobacterium samirii, or is a variant of an enzyme having lactase activity obtained from Bifidobacterium, preferably from Bifidobacterium bifidum or Bifidobacterium samirii.

In another preferred embodiment, the enzyme having lactase activity is obtained from Urmitella, preferably from Urmitella timonensis, or is a variant of an enzyme having lactase activity obtained from Urmitella, preferably from Urmitella timonensis.

In another preferred embodiment, the enzyme having lactase activity is obtained from Streptococcus, preferably from Streptococcus entericus, or is a variant of an enzyme having lactase activity obtained from Streptococcus, preferably from Streptococcus entericus.

In another preferred embodiment, the enzyme having lactase activity is obtained from Varibaculum sp. or is a variant of an enzyme having lactase activity obtained from Varibaculum sp.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

The enzymes may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the enzyme may then be obtained by similarly screening a genomic DNA or cDNA library of another microorganism or mixed DNA sample. Once a polynucleotide encoding an enzyme has been detected with the probe(s), the polynucleotide can be isolated or cloned by utilizing techniques that are known to those of ordinary skill in the art (see, e.g., Davis et al., 2012, Basic Methods in Molecular Biology, Elsevier).

The enzyme having lactase activity may be a polypeptide derived from any of SEQ ID NOs: 1, 2, 4, 5, 6, 7, 8, 9, 10, 11, 12, 13, 14 or 15, or from a mature polypeptide of any of these, wherein the N- and/or C-terminal end has been extended by addition of one or more amino acids or wherein one or more amino acids have been deleted from the N- and/or the C-terminal.

The enzyme having lactase activity may have been C-terminally truncated compared to the wild-type enzyme from which it is obtained.

In a preferred embodiment, the enzyme having lactase activity has a length of at the most about 1500 amino acids, such as at the most about 1400 amino acids or at the most about 1350 amino acids, for example 850-1500 amino acids, preferably 850-1400 amino acids, such as 850-1350 or 1100-1400 amino acids.

In another preferred embodiment, the enzyme having lactase activity has been C-terminally truncated to a length of at the most about 1500 amino acids, such as at the most about 1400 amino acids or at the most about 1350 amino acids, for example 850-1500 amino acids, preferably 850-1400 amino acids, such as 850-1350 or 1100-1400 amino acids.

In one embodiment, the enzyme having lactase activity is obtained from Bifidobacterium, preferably from Bifidobacterium bifidum, or is a variant of an enzyme having lactase activity obtained from Bifidobacterium, preferably from Bifidobacterium bifidum, and has been C-terminally truncated to a length of 850-1500 amino acids, preferably 850-1350 amino acids, more preferably 880-1350, 880-1320 or 885-1310 amino acids.

In another embodiment, the enzyme having lactase activity is obtained from Bifidobacterium, preferably from Bifidobacterium bifidum, or is a variant of an enzyme having lactase activity obtained from Bifidobacterium, preferably from Bifidobacterium bifidum, and has been C-terminally truncated to a length of 850-1500 amino acids, preferably 1250-1500 amino acids, more preferably 1250-1350 or 1290-1350 amino acids, even more preferably 1300-1305 amino acids such as 1302 or 1304 amino acids.

In one embodiment, the enzyme having lactase activity is obtained from Bifidobacterium samirii or is a variant of an enzyme having lactase activity obtained from Bifidobacterium samirii, and has been C-terminally truncated to a length of 850-1500 amino acids, preferably 1250-1400 amino acids, more preferably 1250-1350 or 1280-1320 amino acids, such as 1290-1310 amino acids.

In one embodiment, the enzyme having lactase activity is obtained from Streptococcus entericus DSM 14446 or is a variant of an enzyme having lactase activity obtained from Streptococcus entericus DSM 14446, and has been C-terminally truncated to a length of 850-1500 amino acids, preferably 1250-1400 amino acids, more preferably 1250-1350 or 1270-1310 amino acids, such as 1280-1300 amino acids.

In one embodiment, the enzyme having lactase activity is obtained from Varibaculum sp. or is a variant of an enzyme having lactase activity obtained from Varibaculum sp., and has been C-terminally truncated to a length of 850-1500 amino acids, preferably 1250-1400 amino acids, more preferably 1250-1350 or 1290-1330 amino acids, such as 1300-1320 amino acids.

In one embodiment, the enzyme having lactase activity is obtained from Urmitella timonensis or is a variant of an enzyme having lactase activity obtained from Urmitella timonensis, and has been C-terminally truncated to a length of 850-1500 amino acids, preferably 1250-1400 amino acids, more preferably 1250-1350 or 1290-1330 amino acids, such as 1300-1320 amino acids.

In one embodiment, the enzyme having lactase activity is obtained from Bacillus sp. S3 or is a variant of an enzyme having lactase activity obtained from Bacillus sp. S3, and has been C-terminally truncated to a length of 850-1500 amino acids, preferably 1250-1400 amino acids, more preferably 1250-1350 or 1290-1330 amino acids, such as 1300-1320 amino acids.

In one embodiment, the enzyme having lactase activity is obtained from Bifidobacterium aerophilum or is a variant of an enzyme having lactase activity obtained from Bifidobacterium aerophilum, and has been C-terminally truncated to a length of 850-1500 amino acids, preferably 1250-1400 amino acids, more preferably 1250-1350 or 1280-1320 amino acids, such as 1290-1310 amino acids.

In one embodiment, the enzyme having lactase activity is obtained from Bifidobacterium mongoliense or is a variant of an enzyme having lactase activity obtained from Bifidobacterium mongoliense, and has been C-terminally truncated to a length of 850-1500 amino acids, preferably 1250-1400 amino acids, more preferably 1250-1350 or 1280-1320 amino acids, such as 1290-1310 amino acids.

In one embodiment, the enzyme having lactase activity is obtained from Clostridium nexile CAG: 348 or is a variant of an enzyme having lactase activity obtained from Clostridium nexile CAG: 348, and has been C-terminally truncated to a length of 850-1500 amino acids, preferably 1250-1400 amino acids, more preferably 1250-1350 or 1270-1310 amino acids, such as 1280-1300 amino acids.

In one embodiment, the enzyme having lactase activity is obtained from Neobacillus bataviensis or is a variant of an enzyme having lactase activity obtained from Neobacillus bataviensis, and has been C-terminally truncated to a length of 800-1400 amino acids, preferably 1000-1200 amino acids, more preferably 1100-1200 or 1110-1150 amino acids, such as 1120-1140 amino acids.

In one embodiment, the enzyme having lactase activity is obtained from Neobacillus mesonae or is a variant of an enzyme having lactase activity obtained from Neobacillus mesonae, and has been C-terminally truncated to a length of 850-1500 amino acids, preferably 1250-1400 amino acids, more preferably 1250-1350 or 1290-1330 amino acids, such as 1300-1320 amino acids.

In one embodiment, the enzyme having lactase activity is obtained from Neobacillus niacini or is a variant of an enzyme having lactase activity obtained from Neobacillus niacini, and has been C-terminally truncated to a length of 850-1500 amino acids, preferably 1250-1400 amino acids, more preferably 1250-1350 or 1290-1330 amino acids, such as 1300-1320 amino acids.

In one embodiment, the enzyme having lactase activity is obtained from Streptomyces cirratus or is a variant of an enzyme having lactase activity obtained from Streptomyces cirratus, and has been C-terminally truncated to a length of 1200-1900 amino acids, preferably 1600-1800 amino acids, more preferably 1650-1750 or 1660-1700 amino acids, such as 1670-1690 amino acids.

In a preferred embodiment, the enzyme having lactase activity has an initial lactose turn-over of at least 10 per second per enzyme molecule in milk at 5° C. The initial lactose turn-over may be determined, e.g., when 0-10% of the lactose has been hydrolysed. The skilled person will know how to determine the initial lactose turn-over per enzyme molecule. E.g., it can be a direct measurement of lactose using HPLC or indirect using a glucose detection method.

The initial lactose turn-over per enzyme molecule in milk at 5° C. is about 36 sec⁻¹ for Lactozym® Pure and about 89 sec⁻¹ for Saphera®.

In a more preferred embodiment, the enzyme having lactase activity has an initial lactose turn-over of at least 20, preferably at least 50, more preferably at least 80, per second per enzyme molecule in milk at 5° C.

In a preferred embodiment, the enzyme having lactase activity has an average lactose turn-over from initial lactose, preferably about 4.7% lactose, to 0.1% residual lactose of at least 10 per second per enzyme molecule in milk at 5° C.

The average lactose turn-over from initial lactose of 4.7% to 0.1% residual lactose is about 9 sec⁻¹ for Lactozym® Pure and about 23 sec⁻¹ for Saphera®. The reason for lower average lactose turnover for 4.7% to 0.1% compared to initial rate (4.7% to 4.23%) is a combination of several factors, e.g. higher product inhibition and lower substrate concentration close to 0.1%.

In a preferred embodiment, the enzyme having lactase activity has an average lactose turn-over from initial lactose, preferably about 4.7%, to 0.01% residual lactose of at least 5 per second per enzyme molecule in milk at 5° C.

The average lactose turn-over from initial lactose of 4.7% to 0.01% residual lactose is about 5 sec⁻¹ for Lactozym® Pure and about 18 sec⁻¹ for Saphera®.

In a preferred embodiment, the enzyme having lactase activity has a Michaelis constant K_{M} of at most 40 mM, preferably at most 30 mM, more preferably at most 20 mM, at 5° C.

In another preferred embodiment, the enzyme having lactase activity has a Michaelis constant K_{M} of at most 40 mM, preferably at most 30 mM, more preferably at most 20 mM, at 37° C.

The Michaelis constant K_{M} is the substrate concentration, in this case the lactose concentration, at which the reaction rate is at half-maximum and is a measure of the substrate's affinity for the enzyme. A small K_{M} indicates high affinity, meaning that the rate will approach its maximum with lower substrate concentration than with a larger K_{M}.

The Michaelis constant K_{M} may be determined according to the method of Example 5 of WO09071539A1. In this example, K_{M} at 5° C. is determined as 8 mM for the experimental Bifidobacterium lactase and 30 mM for Lactozym (K. lactis lactase), and K_{M} at 37° C. is determined as 13 mM for the experimental Bifidobacterium lactase and 30 mM for Lactozym.

### PREFERRED EMBODIMENTS

1. A method of producing a lactose-reduced heat-treated milk-based product which comprises:
   a) adding an enzyme having lactase activity to a milk-based substrate comprising at least 2% lactose (w/w),
   b) after addition of the enzyme, performing a heat treatment of the milk-based substrate by holding said milk-based substrate at a holding temperature of at least 120° C. for a holding time of at least 1 second followed by cooling to produce a heat-treated milk-based product, and
   c) storing the heat-treated milk-based product for at least 24 hours, preferably at least 2 days, such as at least 3 days, more preferably at least 4 days, at a temperature of at most 40° C.,
   wherein after step c) the lactose content in the milk-based product is at most 0.2% (w/w).
2. The method of embodiment 1, wherein after step b) but before step c) the lactose content in the milk-based product is at least 0.5% (w/w).
3. The method of any of embodiments 1 or 2, wherein after step a) but before step b), the milk-based substrate is incubated for at most 4 hours, preferably at most 60 minutes, more preferably at most 10 minutes, even more preferably at most 5 minutes.
4. The method of embodiment 3, wherein after step a) but before step b), the milk-based substrate is incubated at a temperature of at most 10° C., preferably at most 7° C.
5. The method of any of embodiments 1 or 2, wherein step b) is performed immediately after step a) without a dedicated incubation step.
6. The method of any of embodiments 1 or 2, wherein step b) is performed immediately after step a) without a dedicated incubation step after step a) and before step b).
7. The method of any of embodiments 1-2 or 5-6, wherein pumping equipment has been fitted to add the enzyme to the milk-based substrate while said milk-based substrate is streaming through process equipment, such as process pipes.
8. The method of any of embodiments 1-2 or 5-7, wherein the time from addition of the enzyme until the holding temperature of step b) is reached is at most 5 minutes, preferably at most 2 minutes, more preferably at most 1 minute.
9. The method of any of embodiments 1-2 or 5-8, wherein the time from addition of the enzyme until the heat-treated milk-based product has been cooled to a temperature of at most 40° C., preferably at most 35° C., more preferably at most 30° C., is at most 3.5 minutes, preferably at most 3 minutes, more preferably at most 2.5 minutes, such as at most 2 minutes or at most 1 minute.
10. The method of any of embodiments 5-9, wherein the enzyme is added to the milk-based substrate while said milk-based substrate is streaming through process equipment, such as process pipes, immediately before the heat treatment step, optionally while the temperature of the milk-based substrate is increasing towards the holding temperature of step b).
11. The method of any of embodiments 1-10, wherein a heating medium is used which is not in direct contact with the milk-based substrate but separated by equipment contact surfaces, preferably wherein the heating medium is a plate heat-exchanger or a tubular heat-exchanger.
12. The method of any of embodiments 1-11, wherein the heat treatment is performed as an indirect heat treatment, preferably an indirect UHT treatment.
13. The method of any of embodiments 1-9, wherein the heat treatment is performed by steam injection or steam infusion, preferably steam injection, using high-pressure steam to heat the milk-based substrate.
14. The method of embodiment 13, wherein step b) is performed immediately after step a) without a dedicated incubation step, and wherein the enzyme is added with the steam.
15. The method of any of embodiments 13-14, wherein after the holding time of step b), the milk-based substrate comprising the steam is flash-cooled in a vacuum to remove water equivalent to amount of condensed steam used.
16. The method of any of embodiments 13-15, wherein the heat treatment is performed as a direct heat treatment, preferably a direct UHT treatment.
17. The method of any of embodiments 13-16, wherein indirect heating using a plate or tubular heat exchanger is also applied.
18. The method of any of the preceding embodiments, wherein the milk-based substrate comprises 2-30%, preferably 2-17%, more preferably 4-5.5%, lactose (w/w).
19. The method of any of the preceding embodiments, wherein the milk-based substrate is milk comprising 4-5.5% lactose (w/w).
20. The method of any of the preceding embodiments, wherein the milk-based substrate is raw milk, preferably raw milk which has not been pasteurized before step a).
21. The method of any of the preceding embodiments, wherein the heat treatment is an ESL treatment, an ultra-pasteurization or a UHT treatment.
22. The method of any of the preceding embodiments, wherein the heat treatment is performed at a temperature of 120-150° C.
23. The method of any of the preceding embodiments, wherein the heat treatment is performed at a temperature of at least 123° C., preferably at 123-145° C.
24. The method of any of the preceding embodiments, wherein the heat treatment is performed at a temperature of at least 130° C., preferably at 130-145° C.
25. The method of any of the preceding embodiments, wherein the heat treatment is performed at a temperature of at least 138° C., preferably at 138-145° C., more preferably at 138-142° C.
26. The method of any of the preceding embodiments, wherein the holding time of step b) is 1-30 seconds, preferably 1-10 seconds, more preferably 1-5 seconds.
27. The method of any of the preceding embodiments, wherein the heat treatment is a UHT treatment, preferably a UHT treatment performed at a temperature of 130-145° C. for a time of 1-30 seconds, more preferably at a temperature of 138-145° C. for a time of 1-10 seconds, even more preferably at a temperature of 138-144° C. for a time of 1-5 seconds.
28. The method of any of the preceding embodiments, wherein the heat treatment is a UHT treatment performed at a temperature of 128-132° C. for 25-35 seconds, at a temperature of 138-140° C. for 2-5 seconds or at a temperature of 144-146° C. for 1-2 seconds.
29. The method of any of embodiments 1-22, wherein the heat treatment is an ESL treatment or an ultra-pasteurization, preferably an ESL treatment or an ultra-pasteurization performed at a temperature of 120-140° C. for a time of 1-5 seconds, more preferably an ESL treatment or an ultra-pasteurization performed at a temperature of 120-130° C. for 1-5 seconds or at a temperature of 138-140° C. for 2-4 seconds.
30. The method of any of the preceding embodiments, wherein no enzyme having lactase activity is added to the milk-based product after step b), preferably after the holding time of step b).
31. The method of any of the preceding embodiments, wherein no enzyme is added to the milk-based product after step b), preferably after the holding time of step b).
32. The method of any of the preceding embodiments, wherein nothing is added to the milk-based product after step b), preferably after the holding time of step b).
33. The method of any of the preceding embodiments, where after the holding time of step b), the milk-based product is cooled to at most 40° C., preferably at most 35° C., more preferably at most 30° C., preferably within 5 minutes, more preferably within 3 minutes, even more preferably within 2 minutes, such as within 1 minute.
34. The method of any of the preceding embodiments, wherein after step b) but before step c) the milk-based product is aseptically packed.
35. The method of any of the preceding embodiments, wherein after step b) but before step c) the milk-based product is homogenized.
36. The method of any of the preceding embodiments, wherein the milk-based product is UHT milk.
37. The method of any of the preceding embodiments, wherein after step b), the enzyme retains at least 0.1% of its initial activity, preferably at least 1%, more preferably at least 2%, more preferably at least 10%, more preferably at least 50%, even more preferably at least 80%, most preferably at least 90%.
38. The method of the preceding embodiment, wherein the initial activity is the activity before step b).
39. The method of any of the preceding embodiments, wherein step c) is performed at a temperature of 2-40° C., preferably 15-40° C., more preferably 18-40° C., most preferably 18-30° C.
40. The method of any of the preceding embodiments, wherein step c) is performed at room temperature which may vary during the storage period.
41. The method of any of the preceding embodiments, wherein after step b) but before step c) the lactose content in the milk-based product is at least 1% (w/w), such as at least 2% (w/w), at least 3% (w/w) or at least 4% (w/w).
42. The method of any of the preceding embodiments, wherein after step b) but before step c) the lactose content in the milk-based product has been reduced by at most 80%, preferably at most 50%, more preferably at most 20%, compared to the lactose content before step a).
43. The method of any of the preceding embodiments, where in step c), the heat-treated milk-based product is stored for at least 7 days, preferably at least 14 days, more preferably at least 21 days.
44. The method of any of the preceding embodiments, wherein after having stored the heat-treated milk-based product for 21 days, preferably 14 days, more preferably 7 days, even more preferably 4 days, most preferably 3 days, at a temperature of 2-40° C., preferably 15-40° C., more preferably 18-40° C., most preferably 18-30° C., the lactose content in the milk-based product is at most 0.2% (w/w), preferably at most 0.1%, more preferably at most 0.01%.
45. The method of any of the preceding embodiments, wherein after having stored the heat-treated milk-based product for 21 days, preferably 14 days, more preferably 7 days, even more preferably 4 days, most preferably 3 days, at a temperature of 2-40° C., preferably 15-40° C., more preferably 18-40° C., most preferably 18-30° C., the lactose content in the milk-based product has been reduced by at least 80% or at least 85%, preferably at least 90, 91, 92, 93, 94, 95, 96 or 97%, more preferably at least 98%, even more preferably at least 99% or at least 99.5%, and most preferably at least 99.8% or at least 99.9%, compared to the lactose content before step a).
46. The method of any of the preceding embodiments, wherein after having stored the heat-treated milk-based product for 21 days, preferably 14 days, more preferably 7 days, even more preferably 4 days, most preferably 3 days, at a temperature of 2-40° C., preferably 15-40° C., more preferably 18-40° C., most preferably 18-30° C., the lactose content in the milk-based product is at most 1000 ppm, preferably at most 100 ppm.
47. The method of any of the preceding embodiments, wherein after step c), the lactose-reduced heat-treated milk product is freeze-dried.
48. The method of any of the preceding embodiments, wherein before step b), a reducing agent is added to the milk-based substrate, preferably a food-approved reducing agent, more preferably a reducing agent selected among L-cysteine, sulphite and glutathione.
49. The method of any of the preceding embodiments, wherein the enzyme having lactase activity is added at a concentration of 100-50,000, preferably 500-40,000 LAU(B) per liter milk-based substrate.
50. The method of any of the preceding embodiments, wherein the enzyme having lactase activity is added at a concentration of 1-150 mg enzyme protein per litre milk-based substrate, preferably 1-100, more preferably 2-50 or 5-50 mg enzyme protein per litre milk-based substrate.
51. The method of any of the preceding embodiments, wherein the enzyme having lactase activity is a lactase.
52. The method of any of the preceding embodiments, wherein the enzyme having lactase activity belongs to enzyme class 3.2.1.21, 3.2.1.23 or 3.2.1.108, preferably 3.2.1.23 or 3.2.1.108.
53. The method of any of the preceding embodiments, wherein the enzyme having lactase activity is a neutral lactase.
54. The method of any of the preceding embodiments, wherein the enzyme having lactase activity is purified.
55. The method of any of the preceding embodiments, wherein the enzyme having lactase activity is isolated.
56. The method of any of the preceding embodiments, wherein the enzyme having lactase activity is a bacterial lactase.
57. The method of any of the preceding embodiments, wherein the enzyme having lactase activity is a GH2 lactase, preferably a GH2 lactase of clade DYLGE.
58. The method of any of the preceding embodiments, wherein the enzyme having lactase activity comprises in its amino acid sequence the motif WTXXDY[I/L/R]GE[P/S/A].
59. The method of any of the preceding embodiments, wherein the enzyme having lactase activity comprises in its amino acid sequence the motif(s) SR[W/Y/F]YSGSGX[Y/G]R and/or [L/V/I]X[L/V/I]PHD.
60. The method of any of the preceding embodiments, wherein the enzyme having lactase activity has a CBM32 or CBM71, preferably a CBM32.
61. The method of any of the preceding embodiments, wherein the enzyme having lactase activity has a temperature optimum of 30-60° C., preferably 35-55° C.
62. The method of any of the preceding embodiments, wherein the enzyme having lactase activity has a melting temperature Tm of 50-70° C. determined by thermal shift at pH 6.
63. The method of any of the preceding embodiments, wherein the enzyme having lactase activity has a melting temperature Tm of 50-70° C. determined by thermal shift at pH 7.
64. The method of any of the preceding embodiments, wherein the enzyme having lactase activity has a residual activity of at least 1%, preferably at least 2%, more preferably at least 5%, even more preferably at least 10%, after incubation at 70° C. for 30 seconds and 140° C. for 5 seconds in skimmed milk having a lactose content of 4.7%.
65. The method of any of the preceding embodiments, wherein the enzyme having lactase activity has a residual activity of at least 0.1%, preferably at least 0.5%, at least 1% or at least 2%, more preferably at least 5%, even more preferably at least 10%, after incubation in skimmed milk having a lactose content of 4.7% at 90° C. for 30 seconds, at 140° C. for 5 seconds and at 70° C. for 30 seconds followed by cooling to 0-10° C. and subsequent incubation at 23° C. for 0.5 hours, wherein the residual activity is relative to the activity of the same enzyme in skimmed milk without incubation at 90° C. for 30 seconds, at 140° C. for 5 seconds and at 70° C. for 30 seconds followed by cooling to 0-10° C. and subsequent incubation at 23° C. for 0.5 hours.
66. The method of any of the preceding embodiments, wherein the enzyme having lactase activity has a residual activity of at least 0.5%, preferably at least 1%, at least 2% or at least 3%, more preferably at least 5%, even more preferably at least 10%, after incubation in skimmed milk having a lactose content of 4.7% at 90° C. for 30 seconds, at 140° C. for 5 seconds and at 70° C. for 30 seconds followed by cooling to 0-10° C. and subsequent incubation at 23° C. for 72 hours, wherein the residual activity is relative to the activity of the same enzyme in skimmed milk without incubation at 90° C. for 30 seconds, at 140° C. for 5 seconds and at 70° C. for 30 seconds followed by cooling to 0-10° C. and subsequent incubation at 23° C. for 72 hours.
67. The method of any of the preceding embodiments, wherein the enzyme having lactase activity has an activity after incubation in skimmed milk having a lactose content of 4.7% at 90° C. for 30 seconds, at 140° C. for 5 seconds and at 70° C. for 30 seconds followed by cooling to 0-10° C. and subsequent incubation at 23° C. for 72 hours, which is at least 20% higher than the activity after incubation in skimmed milk having a lactose content of 4.7% at 90° C. for 30 seconds, at 140° C. for 5 seconds and at 70° C. for 30 seconds followed by cooling to 0-10° C. and subsequent incubation at 23° C. for 0.5 hours.
68. The method of any of the preceding embodiments, wherein the enzyme having lactase activity has an amino acid sequence which is at least 50%, such as at least 60%, at least 70%, at least 80%, at least 90%, at least 95%, at least 98%, at least 99%, at least 99.5% or 100%, identical to any of SEQ ID NO: 1, SEQ ID NO: 2, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15 or to a mature polypeptide of any of these.
69. The method of any of the preceding embodiments, wherein the enzyme having lactase activity has an amino acid sequence which is at least 50%, such as at least 60%, at least 70%, at least 80%, at least 90%, at least 95%, at least 98%, at least 99%, at least 99.5% or 100%, identical to any of SEQ ID NO: 4, SEQ ID NO: 5, SEQ ID NO: 6 or SEQ ID NO: 11 or to a mature polypeptide of any of these.
70. The method of any of the preceding embodiments, wherein the enzyme having lactase activity is selected from the group consisting of:
   a) an enzyme having an amino acid sequence which is at least 50%, such as at least 60%, at least 70%, at least 80%, at least 90%, at least 95%, at least 98%, at least 99%, at least 99.5% or 100%, identical to SEQ ID NO: 1 and comprising at least one amino acid substitution selected from the group consisting of P65A, N275S, C372A, M386Q, R389A, G482A, T523N, P615T, T756K, A936S, T972C, I1035C, T1076C, Y1122C, A1129C, F1132C, K1168C, S1195C and C1199S, for example two or more of said amino acid substitutions, wherein numbering is based on SEQ ID NO: 1,
   b) an enzyme having an amino acid sequence which is at least 50%, such as at least 60%, at least 70%, at least 80%, at least 90%, at least 95%, at least 98%, at least 99%, at least 99.5% or 100%, identical to SEQ ID NO: 4 and comprising at least one amino acid substitution selected from the group consisting of P65A, C372A, P615T, A1073C, H1122C and C1195G, for example two or more of said amino acid substitutions, wherein numbering is based on SEQ ID NO: 4,
   c) an enzyme having an amino acid sequence which is at least 50%, such as at least 60%, at least 70%, at least 80%, at least 90%, at least 95%, at least 98%, at least 99%, at least 99.5% or 100%, identical to SEQ ID NO: 11 and comprising at least one amino acid substitution selected from the group consisting of P52A, G607T, L1064C and Y1110C, for example two or more of said amino acid substitutions, wherein numbering is based on SEQ ID NO: 11, and
   d) an enzyme having an amino acid sequence which is at least 50%, such as at least 60%, at least 70%, at least 80%, at least 90%, at least 95%, at least 98%, at least 99%, at least 99.5% or 100%, identical to SEQ ID NO: 5 and comprising the substitution G386Q and/or P620T, wherein numbering is based on SEQ ID NO: 5.
71. The method of any of the preceding embodiments, wherein the enzyme having lactase activity has an amino acid substitution of the cysteine corresponding to C372 of SEQ ID NO: 1, preferably to serine, alanine or glycine.
72. The method of any of the preceding embodiments, wherein the enzyme having lactase activity is obtained from Bifidobacterium, preferably from Bifidobacterium bifidum, Bifidobacterium samirii, Bifidobacterium aerophilum or Bifidobacterium mongoliense, or from Bacillus, preferably from Bacillus circulans or Bacillus sp. S3, from Varibaculum sp., from Urmitella, preferably from Urmitella timonensis, from Streptococcus, preferably from Streptococcus entericus DSM 14446, from Streptomyces, preferably from Streptomyces cirratus, from Clostridium, preferably from Clostridium nexile CAG: 348, or from Neobacillus, preferably from Neobacillus bataviensis, Neobacillus mesonae or Neobacillus niacini, or is a variant of an enzyme obtained from any of these genera or species.
73. The method of any of the preceding embodiments, wherein the enzyme having lactase activity has a length of at the most about 1500 amino acids, such as at the most about 1400 amino acids or at the most about 1350 amino acids, for example 850-1500 amino acids, preferably 850-1400 amino acids, such as 850-1350 or 1100-1400 amino acids.
74. The method of any of the preceding embodiments, wherein the enzyme having lactase activity has an amino acid sequence which is at least 50%, such as at least 60%, at least 70%, at least 80%, at least 90%, at least 95%, at least 98%, at least 99%, at least 99.5% or 100% identical to any of SEQ ID NOs: 1 or 2 or to a mature polypeptide of any of SEQ ID NOs: 1 or 2.
75. The method of any of the preceding embodiments, wherein the enzyme having lactase activity has an amino acid sequence which is at least 50%, such as at least 60%, at least 70%, at least 80%, at least 90%, at least 95%, at least 98%, at least 99%, at least 99.5% or 100% identical to SEQ ID NO: 1.
76. The method of any of the preceding embodiments, wherein the enzyme having lactase activity is a polypeptide derived from any of SEQ ID NOs: 1, 2, 3, 4, 5, 6, 7, 8, 9, 10, 11, 12, 13, 14 or 15 or from a mature polypeptide of any of these having 1-30 alterations, e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions.
77. The method of any of the preceding embodiments, wherein the enzyme having lactase activity is a polypeptide derived from SEQ ID NO: 1 having 1-30 alterations, e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions.
78. The method of any of the preceding embodiments, wherein the enzyme having lactase activity is a polypeptide derived from any of SEQ ID NOs: 1, 2, 3, 4, 5, 6, 7, 8, 9, 10, 11, 12, 13, 14 or 15 or from a mature polypeptide of any of these wherein the N- and/or C-terminal end has been extended by addition of one or more amino acids or wherein one or more amino acids have been deleted from the N- and/or the C-terminal.
79. The method of any of the preceding embodiments, wherein the enzyme having lactase activity has been C-terminally truncated compared to the wild-type enzyme from which it is obtained.
80. The method of any of the preceding embodiments, wherein the enzyme having lactase activity is obtained from Bifidobacterium, preferably from Bifidobacterium bifidum, or from Bacillus, preferably from Bacillus circulans, or is a variant of an enzyme having lactase activity obtained from Bifidobacterium, preferably from Bifidobacterium bifidum, or from Bacillus, preferably from Bacillus circulans.
81. The method of any of the preceding embodiments, wherein the enzyme having lactase activity is obtained from Bifidobacterium, preferably from Bifidobacterium bifidum, or is a variant of an enzyme having lactase activity obtained from Bifidobacterium, preferably from Bifidobacterium bifidum.
82. The method of the preceding embodiment, wherein the enzyme having lactase activity has a length of 850-1500 amino acids, preferably 850-1350 amino acids, more preferably 880-1350, 880-1320 or 885-1310 amino acids.
83. The method of any of the two preceding embodiments, wherein the enzyme having lactase activity has a length of 850-1500 amino acids, preferably 1250-1500 amino acids, more preferably 1250-1350 or 1290-1350 amino acids, even more preferably 1300-1305 amino acids such as 1302 or 1304 amino acids.
84. The method of any of the preceding embodiments, wherein the enzyme having lactase activity has an initial lactose turn-over of at least 10 per second per enzyme molecule in milk at 5° C.
85. The method of any of the preceding embodiments, wherein the enzyme having lactase activity has an initial lactose turn-over of at least 20, preferably at least 50, more preferably at least 80, per second per enzyme molecule in milk at 5° C.
86. The method of any of the preceding embodiments, wherein the enzyme having lactase activity has an average lactose turn-over from initial lactose, preferably about 4.7% lactose, to 0.1% residual lactose of at least 10 per second per enzyme molecule in milk at 5° C.
87. The method of any of the preceding embodiments, wherein the enzyme having lactase activity has an average lactose turn-over from initial lactose, preferably about 4.7%, to 0.01% residual lactose of at least 5 per second per enzyme molecule in milk at 5° C.
88. The method of any of the preceding embodiments, wherein the enzyme having lactase activity has a Michaelis constant K_{M} of at most 40 mM, preferably at most 30 mM, more preferably at most 20 mM, at 5° C.
89. The method of any of the preceding embodiments, wherein the enzyme having lactase activity has a Michaelis constant K_{M} of at most 40 mM, preferably at most 30 mM, more preferably at most 20 mM, at 37° C.

### EXAMPLES

### Method 1

### LAU(B) Assay

The activity in LAU-B/g of a specific lactase may be determined by direct measurement of o-nitrophenyl (ONP) released from o-nitrophenyl β-D-galactopyranoside (ONPG) in a buffer containing 1.46 mg/ml substrate in 0.05 M MES, 1 mM MgSO4 7H2O, 450 mg/L Brij 35 at pH 6.5 and 30° C. After 600 seconds incubation, the reaction is stopped by adding 0.2 M Na2CO3 and the released ONP is measured at 405 nm after 126 seconds incubation. The activity is obtained by comparing to a standard curve run with a lactase of known activity, and the activity of the unknown sample calculated from this. The lactase of known activity may, e.g., be Saphera® obtained from Novozymes A/S, Denmark.

### Method 2

### Assay for Determining Temperature Optimum

### Assay for Determining Temperature Profile 35° C.-75° C.

Temperature profile to determine the temperature optimum is prepared by adding 10 μl diluted enzyme samples (diluted with 50 mM succinate, 50 mM HEPES, 50 mM CHES, 150 mM KCl, 2 mM CaCl2, 1 mM MgCl2+0.01% triton X-100, pH 6.5) to PCR tubes. Then 90 μl substrate (167 mM lactose, 50 mM succinate, 50 mM HEPES, 50 mM CHES, 150 mM KCl, 2 mM CaCl2, 1 mM MgCl2, pH 6.5) is added and the PCR tubes is placed in a preheated PCR block with temperature gradient 35-75° C. (using TProfessional thermocycler, Biometra) and incubated for 30 min at 35-75° C. (gradient), and then placed on ice. The reaction is stopped by adding 100 μl 0.25 M NaOH. Twenty μl is transferred to a 96 well microtiter plate, and 230 μL GOD-Perid (100 mM potassium phosphate buffer, pH 7, 0.6 g/l Glucose oxidase, 0.02 g/l horseradish peroxidase, 1.0 g/l ABTS) solution is added. After 30 minutes in the dark at room temperature the absorbance is measured at 420 nm. The initial dilution of the enzyme should be adjusted so the final 420 nm reading at the optimum temperature is between abs. 0.5-2.5. The temperature with the highest delta Abs at 420 nm (“Abs 420 nm with enzyme” minus “Abs 420 nm without enzyme”, i.e. background) is set to 100% and the reaming delta Abs 420 nm is then related to the delta Abs 420 nm with the highest value.

### Example 1

The lactase used is Saphera 2600L (Bifidobacterium bifidum, SEQ ID NO: 1) from Novozymes A/S having a declared activity of 2600 LAU(B)/g.

### Test 1

248.5 liter of skimmed milk was pumped into an incubation tank, and then cooled down until 5° C.

1.5 kg of Saphera was added to the milk (0.6% dosage corresponding to 15,600 LAU(B) per liter milk), and then milk was agitated at ca. 50 rpm for 18 h.

At the end of incubation, the milk was processed under UHT using steam injection: UHT conditions (140° C./4 sec), and downstream 2-stage homogenization (70° C. 200/50 bar).

The milk was cooled from 140 degrees to ca. 90 degrees in a flash cooling manner which means that the milk temperature is instantly (about 1 sec) decreased to 90 degrees. After this, it took about 30 sec to decrease the temperature to about 60 degrees (including downstream homogenization) and then it took again about 30 sec to decrease the temperature to room temperature (about 20° C.) before filling.

Samples were collected after UHT; 2 samples were frozen until analysis, and the rest of the samples were stored at room temperature up to 3 weeks and analyzed for residual lactose.

### Test 2

248.875 liter of skimmed milk was mixed well at cold temperature (5° C.) with 1.125 kg of Saphera (0.45% dosage). The enzyme and milk were mixed for 45 min and then treated under UHT conditions using steam injection: UHT conditions (140° C./4 sec), and downstream 2-stage homogenization (70° C. 200/50 bar).

The milk was cooled from 140 degrees to ca. 90 degrees in about 1 sec using flash cooling. After this, it took about 30 sec to decrease the temperature to about 60 degrees (including downstream homogenization) and then it took again about 30 sec to decrease the temperature to room temperature (about 20° C.) before filling.

Samples were collected after UHT; 2 samples were frozen until analysis, and the rest of the samples were stored at room temperature up to 3 weeks and analyzed for residual lactose.

### Test 3

248.5 liter of skimmed milk was pumped to the UHT unit. When the temperature of milk reached 70° C., 1.5 kg of Saphera (0.6% dosage) was pumped to the hot milk stream (70° C.) while the milk stream was on its way to the UHT treatment. The UHT conditions were steam injection: UHT conditions (140° C./4 sec) and downstream 2-stage homogenization (70° C. 200/50 bar).

After addition of the enzyme, the milk got in touch with the hot steam instantly which raises the temperature of the milk immediately (about 1 sec) to 140 degrees, followed by 4 sec holding time at 140 degrees, 1 sec (flash cooling) to decrease the temperature to ca. 90 degrees, 30 sec to decrease the temperature further down to about 60 degrees (during this stage, the downstream homogenization was performed), 30 sec to decrease the temperature to room temperature (about 20° C.) before filling.

Samples were collected after UHT; 2 samples were frozen until analysis, and the rest of the samples were stored at room temperature up to 3 weeks and analyzed for residual lactose.

### Test 4

248.5 liter of skimmed milk was mixed well at cold temperature (5° C.) with 1.5 kg of Saphera (0.6% dosage). The enzyme and milk were mixed for 10 min and then treated under UHT conditions using steam injection: UHT conditions (140° C./4 sec), and downstream 2-stage homogenization (70° C. 200/50 bar).

The milk was cooled from 140 degrees to ca. 90 degrees in about 1 sec using flash cooling. After this, it took about 30 sec to decrease the temperature to about 60 degrees (including downstream homogenization) and then it took again about 30 sec to decrease the temperature to room temperature (about 20° C.) before filling.

Samples were collected after UHT; 2 samples were frozen until analysis, and the rest of the samples were stored at room temperature up to 3 weeks and analyzed for residual lactose.

### Test 5

247 liter of skimmed milk was mixed well at cold temperature (5° C.) with 3 kg of Saphera (1.2% dosage). Milk was treated instantly under UHT conditions using steam injection: UHT conditions (140° C./4 sec), and downstream 2-stage homogenization (70° C. 200/50 bar).

The milk was cooled from 140 degrees to ca. 90 degrees in about 1 sec using flash cooling. After this, it took about 30 sec to decrease the temperature to about 60 degrees (including downstream homogenization) and then it took again about 30 sec to decrease the temperature to room temperature (about 20° C.) before filling.

Samples were collected after UHT; 2 samples were frozen until analysis, and the rest of the samples were stored at room temperature up to 3 weeks and analyzed for residual lactose.

### Test 6

249.25 liter of skimmed milk was mixed well at cold temperature (5° C.) with 0.75 kg of Saphera (0.3% dosage). Milk was treated instantly under UHT conditions using steam injection: UHT conditions (140° C./4 sec), and downstream 2-stage homogenization (70° C. 200/50 bar).

The milk was cooled from 140 degrees to ca. 90 degrees in about 1 sec using flash cooling. After this, it took about 30 sec to decrease the temperature to about 60 degrees (including downstream homogenization) and then it took again about 30 sec to decrease the temperature to room temperature (about 20° C.) before filling.

Samples were collected after UHT; 2 samples were frozen until analysis, and the rest of the samples were stored at room temperature up to 3 weeks and analyzed for residual lactose.

### Test 7

247 liter of skimmed milk was pumped to the UHT unit. When the temperature of milk reached 90° C., the milk was incubated for 2 min at 90° C. in a holding tube. Then, 3 kg of Saphera (1.2% dosage) was pumped to the hot milk stream (90° C.) while the milk stream was on its way to the UHT treatment. The UHT conditions were tube exchange heating: UHT conditions (140° C./4 sec). Here the 2-stage homogenization was done upstream (70° C. 200/50 bar).

After addition of the enzyme, it took about 30 sec to heat up to 140 degrees, 4 sec holding time at 140 degrees, about 40 sec to decrease the temperature to about 45 degrees and then another 30 sec to decrease the temperature to room temperature (about 20° C.) before filling.

Samples were collected after UHT; 2 samples were frozen until analysis, and the rest of the samples were stored at room temperature up to 3 weeks and analyzed for residual lactose.

### Analysis of Residual Lactose Content

The analysis of residual lactose was performed by high-performance anion exchange chromatography coupled with pulsed amperometry detector (HPAEC-PAD).

### Sample Preparation for HPAEC-PAD:

50 μl sample was transferred to 5 ml Eppendorf tube containing 500 μl MQ water. 10 ul Carrez I solution was added and mixed, and then 10 ul Carrez II solution was added and mixed. Then, 4.43 ml MQ water (total volume of 5 ml) was added and mixed. Centrifugation was carried out at 14200 rpm for 5 min. The supernatant was diluted ×5 and later ×20 for some samples. These samples were analyzed on HPAEC-PAD.

### Analytical Method:

Equipment: Dionex IC-5000
Pump: ICS-5000 Single pump model SP-5, S/N 11113787
Autosampler: Thermo Scientific AS-AP, P/N 074925, S/N 12101147
Oven: ICS-5000 Detector/Chromatography module model DC-5, S/N: 11102695
Detector: Dionex IC-5000 ECD, P/N 072043, S/N 11113974, ECD cell P/N 072044, S/N 10198 with non-disposable gold electrode P/N 063494, S/N 50435.
Columns: CarboPac PA20 column, 3×150 mm (Dionex P/N 060142) coupled with CarboPac PA20 Guard column, 3×30 mm (Dionex P/N 060144)

### Eluents:

A: MQ water
B: 200 mM NaOH
C: 100 mM Sodium Acetate in 200 mM NaOH
D: 1 mM Sodium Acetate in 200 mM NaOH

TABLE 1
Gradient program:

| No | Time | Flow (ml/min) | % B | % C | % D | Curve |
|---|---|---|---|---|---|---|
| 1 | 0.000 | Run | | | | |
| 2 | 10.000 | 0.400 | 6.0 | 0.0 | 0.0 | 5 |
| 3 | 20.000 | 0.400 | 12.0 | 0.0 | 0.0 | 5 |
| 4 | 25.000 | 0.400 | 12.0 | 0.0 | 0.0 | 5 |
| 5 | 30.000 | 0.400 | 50.0 | 5.0 | 0.0 | 5 |
| 6 | 36.500 | 0.400 | 50.0 | 5.0 | 0.0 | 5 |
| 7 | 38.000 | 0.400 | 50.0 | 0.0 | 50.0 | 5 |
| 8 | 44.000 | 0.400 | 50.0 | 0.0 | 50.0 | 5 |
| 9 | 44.100 | 0.400 | 100.0 | 0.0 | 0.0 | 5 |
| 10 | 49.000 | 0.400 | 100.0 | 0.0 | 0.0 | 5 |
| 11 | 49.100 | 0.400 | 6.0 | 0.0 | 0.0 | 5 |
| 12 | 70.000 | 0.400 | 6.0 | 0.0 | 0.0 | 5 |
| 13 | New row | | | | | |

| | | |
|---|---|---|
| 14 | 70.000 | Stop Run |

TABLE 2
Results:

| | | Enzyme | Enzyme | | | Storage | | |
|---|---|---|---|---|---|---|---|---|
| | Enzyme | added to | added to | UHT | | time (days) | | |
| Trial | dosage | cold | hot milk | tech- | UHT | at room | | Lactose |
| ID | % | milk | stream | nology | conditions | temperature | | content % |
| Fresh | / | / | / | / | / | / | | 5.3 ± 0.08 |
| milk | | | | | | | | |
| Test 1 | 0.6% | Yes @ | / | Steam | 140° C./4 sec | 0 | days | 0.027 ± 0.004 |
| | | 5° C. for | | injection | Downstream | 4 | days | 0.006 ± 0.0005 |
| | | 18 h | | | homogeni- | 7 | days | 0.006 ± 0.0008 |
| | | | | | zation | 14 | days | 0.003 ± 0.0001 |
| | | | | | | 21 | days | 0.002 |
| Test 2 | 0.45% | Yes @ | / | Steam | 140° C./4 sec | 0 | days | 1.54 ± 0.08 |
| | | 5° C. for | | injection | Downstream | 4 | days | 0.31 ± 0.02 |
| | | 45 min | | | homogeni- | 7 | days | 0.12 ± 0.002 |
| | | | | | zation | 14 | days | 0.032 ± 0.004 |
| | | | | | | 21 | days | 0.017 ± 0.002 |
| Test 3 | 0.6% | / | Yes @ | Steam | 140° C./4 sec | 0 | days | 4.51 ± 0.09 |
| | | | 70° C. | injection | Downstream | 4 | days | 1.08 ± 0.14 |
| | | | | | homogeni- | 7 | days | 0.34 ± 0.04 |
| | | | | | zation | 14 | days | 0.076 ± 0.002 |
| | | | | | | 21 | days | 0.033 |
| Test 4 | 0.6% | Yes @ | / | Steam | 140° C./4 sec | 0 | days | 2.14 ± 0.03 |
| | | 5° C. for | | injection | Downstream | 4 | days | 0.26 ± 0.002 |
| | | 10 min | | | homogeni- | 7 | days | 0.075 ± 0.004 |
| | | | | | zation | 14 | days | 0.023 ± 0.0009 |
| | | | | | | 21 | days | 0.013 |
| Test 5 | 1.2% | Yes @ | / | Steam | 140° C./4 sec | 0 | days | 0.83 ± 0.02 |
| | | 5° C. for | | injection | Downstream | 4 | days | 0.042 ± 0.0005 |
| | | 5 min | | | homogeni- | 7 | days | 0.075 ± 0.004 |
| | | | | | zation | 14 | days | 0.007 ± 0.00007 |
| | | | | | | 21 | days | 0.0035 ± 0.0005 |
| Test 6 | 0.3% | Yes @ | / | Steam | 140° C./4 sec | 0 | days | 3.68 ± 0.02 |
| | | 5° C. for | | injection | Downstream | 4 | days | 1.04 ± 0.009 |
| | | 5 min | | | homogeni- | 7 | days | 0.2 ± 0.02 |
| | | | | | zation | 14 | days | 0.038 ± 0.0005 |
| | | | | | | 21 | days | 0.017 |
| Test 7 | 1.2% | / | Yes @ | Tube ex- | 140° C./4 sec | 0 | days | 4.68 ± 0.03 |
| | | | 90° C. | change | Downstream | 4 | days | 0.82 ± 0.05 |
| | | | | | homogeni- | 7 | days | 0.11 ± 0.005 |
| | | | | | zation | 14 | days | 0.016 ± 0.001 |
| | | | | | | 21 | days | 0.0075 ± 0.0005 |

### Example 2

### UHT Treatment

1% (w/v) Saphera 2600L (Bifidobacterium bifidum, SEQ ID NO: 1), 0.25% (w/v) Biolacta N5 (Bacillus circulans, SEQ ID NO: 2), 1% (w/v) Bonlacta (Lactobacillus delbrueckii subsp. Bulgaricus, SEQ ID NO: 3) and 1% (w/v) Lactozym Pure 6500L (Kluyveromyces lactis) were each added to skimmed milk having a lactose content of 4.7% and sodium azide* was added to a final conc. of 0.025% (w/v) to avoid microbial growth as handling/tubes etc. is not fully sterile despite having a UHT step. These milk samples were applied to a lab. scale UHT setup as described below. A syringe with 10 ml milk sample was connected to a long Teflon tube that was immersed sequentially in three baths. The first bath with silicone had 70° C. and 3 m of the Teflon tube, the 2nd connected bath also with silicone had 140° C. and 50 cm of the Teflon tube, whereas the final bath was an ice/water bath (0° C.) containing 1 m to cool the milk after the 140° C. treatment. A flow of 3 ml/min was applied on the syringe which ensures that the milk samples were incubated at 70° C. for 30 sec and 140° C. for 5 sec before cooling for 10 sec in the ice/water bath so that the milk is cooled to 0-10° C. Finally, the milk was collected in a 10 ml syringe after the ice/water bath, and the samples were assayed for residual activity. * Sodium azide is not to be used in a commercial process where sterility is secured, but is used in this lab set-up which may not be completely sterile and where the milk is not to be consumed.

### Residual Activity Assay

Samples were centrifuged at 20,600 g in a precooled centrifuge for 45 min at 5° C. and dilution with 20 mM sodium succinic acid and 0.01% triton x-100, pH 6.5 of the supernatant was made so an absorbance reading below 1.5 at 405 nm could be measured. Twenty five μl of each sample was mixed with 175 μl of ONPG substrate (1.67 mg/ml ONPG (o-NitroPhenyl β-D-galatopyranoside, ˜5.5 mM), 0.05 M MES, 1 mM MgSO4, 150 mM KCl, 0.01% Triton X-100, pH 6.5) and incubated for 2.5 hr at 40° C. and stopped by adding 50 μl Na2CO3+5 mM Na4EDTA and measured at 405 nm. Residual activity in % was calculated using the formulae=((Abs405^{heat-treated sample}−Abs405^{blank})*dilution factor)/((Abs405^{untreated sample}−Abs405^{blank})*dilution factor)*100%.

The heat-treated sample is enzyme mixed with skimmed milk and sodium azide and subjected to UHT treatment and subsequent cooling as described above. The untreated sample is the same enzyme mixed with skimmed milk and sodium azide but without UHT treatment. Both of these samples were diluted to obtain an absorption at 405 nm in the range of 0.5-1.0. The blank is sample without enzyme and the same dilution factor has been used as for the sample with enzyme.

TABLE 3
Results:

| | Residual |
|---|---|
| | activity % |
| Saphera 2600L | 2.14 |
| Biolacta N5 | 24.75 |
| Bonlacta | 0.02 |
| Lactozym pure 6500L | 0.03 |

Table 3 shows that lactase can survive the UHT treatment to various degrees, where most residual activity can be found with the following ranking (highest residual activity first) Biolacta N5, Saphera 2600L, Lactozym pure 6500L and Bonlacta.

Biolacta® N5 (SEQ ID NO: 2) and Saphera® 2600L (SEQ ID NO: 1) are GH2 lactases of clade DYLGE, whereas Lactozym® Pure 6500L (yeast lactase from Kluyveromyces lactis) and Bonlacta® (SEQ ID NO: 3) are GH2 lactases of clade MGN.

### Example 3

### UHT Treatment

Batches of PE variants of Saphera (SEQ ID NO: 1) having the amino acid substitutions shown in Table 4 were added to a final conc. of 5% (v/v) in skimmed milk with 0.025% sodium azide. The 5% (v/v) roughly corresponds to the same amount of enzyme as 1% (w/v) of Saphera 2600L. These milk samples were applied to a lab. scale UHT setup as described below. A syringe with 10 ml milk sample was connected to a long Teflon tube that was immersed sequentially in three baths. The first bath with silicone had 70° C. and 3 m of the Teflon tube, the 2^{nd} connected bath also with silicone had 140° C. and 50 cm of the Teflon tube, whereas the final bath was an ice/water bath (0° C.) containing 1 m to cool the milk after the 140° C. treatment. A flow of 3 ml/min was applied on the syringe which ensures that the milk samples were incubated at 70° C. for 30 sec and 140° C. for 5 sec before cooling for 10 sec in the ice/water bath so that the milk is cooled to 0-10° C. Finally, the milk was collected in a 10 ml syringe after the ice/water bath, and the samples were assayed for residual activity.

### Residual Activity Assay

Samples were centrifuged at 20,600 g in a precooled centrifuge for 45 min at 5° C. and dilution with 20 mM sodium succinic acid and 0.01% triton x-100, pH 6.5 of the supernatant was made so an absorbance reading below 1.5 at 405 nm could be measured. Twenty five μl of each sample was mixed with 175 μl of ONPG substrate (1.67 mg/ml ONPG (o-NitroPhenyl β-D-galatopyranoside, ˜5.5 mM), 0.05 M MES, 1 mM MgSO4, 150 mM KCl, 0.01% Triton X-100, pH 6.5 (preferably adjusted with NaOH)) and incubated for 2.5 hr at 40° C. and stopped by adding 50 μl Na2CO3+5 mM Na4EDTA and measured at 405 nm. Residual activity in % was calculated using the formulae=((Abs405^{heat-treated sample}−Abs405^{blank})*dilution factor)/((Abs405^{untreated sample}−Abs405^{blank})*dilution factor)*100%.

Then the residual activity of each variant was related to the residual activity of the lactase of SEQ ID NO: 1 using the following formulae=(% residual activity of a variant)/(% residual activity of lactase of SEQ ID NO: 1)*100% and these numbers are shown in Table 4.

TABLE 4
Results:

| Variant | | % residual activity |
|---|---|---|
| (V) no. | Amino acid substitutions | compared to SEQ1 |
| V1 | A1129C S1195C | 150 |
| V2 | R389A G482A T523N T756K A1129C S1195C | 165 |
| V3 | K274L R389A G482A T523N T756K A936S T972C | 118 |
| | I1035C A1129C S1195C | |
| V4 | N275S R389A G482A T523N T756K A936S T972C | 170 |
| | I1035C A1129C S1195C | |
| V5 | N275S R389A G482A T523N T756K A1129C S1195C | 146 |
| V6 | R389A G482A T523N T756K A936S T972C I1035C | 269 |
| | A1129C S1195C | |
| V7 | N275S R389A G482A T523N T756K A936S T972C | 154 |
| | I1035C A1129C F1132C K1168C S1195C | |
| V8 | R389A G482A T523N T756K A936S T972C I1035C | 332 |
| | A1129C F1132C K1168C S1195C | |
| V9 | K274L R389A G482A T523N T756K A1129C F1132C | 618 |
| | K1168C S1195C | |
| V10 | N275S R389A G482A T523N T756K A1129C F1132C | 248 |
| | K1168C S1195C | |
| SEQ1 | none | 100 |

Table 4 shows that all ten variants V1-V10 have improved residual activity compared to the lactase of SEQ ID NO: 1. Especially V9 had more than 6-fold higher residual activity compared to the lactase of SEQ ID NO: 1.

### Example 4

Newly Identified Lactases Having a High Residual Activity after UHT Treatment

In this example, a number of newly identified lactases and variants thereof are demonstrated to be useful in the method of the present invention. The lactases are added to the milk just before UHT treatment and have been shown to have sufficient residual activity after the UHT treatment to efficiently hydrolyse the lactose in the milk during a few days of storage at room temperature.

Some of the lactases are newly identified wild-type enzymes which have been C-terminally truncated to facilitate expression as secreted enzymes, while others are variants of these wild-type enzymes in which one or more amino acids have been substituted using established protein engineering (PE) techniques. The enzymes tested in this example are listed in Table 5 below and a matrix of the sequence identities between the truncated wild-type enzymes is shown in Table 6.

Some of the newly identified C-terminally truncated wild-type enzymes are further described in co-pending application(s) claiming priority from EP22215776 filed 22 Dec. 2022.

Expression in Bacillus subtilis

The genome sequences of the bacterial donor strains (Table 5) were downloaded from EMBL-EBI (www.ebi.ac.uk/) and analyzed for putative lactases from the CAZY database GH2 family (Lombard V, Golaconda Ramulu H, Drula E, Coutinho P M, Henrissat B (2014) The Carbohydrate-active enzymes database (CAZy), 2013, Nucleic Acids Res 42: D490-D495; Elodie Drula, Marie-Line Garron, Suzan Dogan, Vincent Lombard, Bernard Henrissat, Nicolas Terrapon, The carbohydrate-active enzyme database: functions and literature, Nucleic Acids Research, Volume 50, Issue D1, 7 Jan. 2022, Pages D571-D577; http://www.cazy.org/).

The genes encoding the enzymes of Table 5 were optimized for expression in Bacillus subtillis using standard methods known in the art. Alternatively, codon-optimized genes can be purchased commercially. For the lactases of SEQ ID NO: 9 (Neobacillus bataviensis) and SEQ ID NO: 12 (Streptomyces cirratus), the natural DNA (of the truncated mature peptide) was used. The genes were fused with DNA encoding a Bacillus clausii secretion signal (encoding the following amino acid sequence: MKKPLGKIVASTALLISVAFSSSIASA (SEQ ID NO: 16)) replacing the native secretion signal. Furthermore, the expression construct results in the addition of an amino-terminal poly histidine tag consisting of the amino acid sequence HHHHHHPR (SEQ ID NO: 17)) to the mature lactase to facilitate easy purification by immobilized metal affinity chromatography. The resulting gene was ordered as a fully synthetically produced DNA fragment from Twist Bioscience (San Francisco, CA, USA).

The linear integration constructs were SOE-PCR fusion products (Horton, R. M., Hunt, H. D., Ho, S. N., Pullen, J. K. and Pease, L. R. (1989) Engineering hybrid genes without the use of restriction enzymes, gene splicing by overlap extension, Gene 77:61-68) made by fusion of the gene of interest between two B. subtilis chromosomal regions along with strong promoters and a chloramphenicol resistance marker. The SOE-PCR method is also described in patent application WO2003095658.

The lactase genes were expressed under the control of a triple promoter system (as described in WO 99/43835), consisting of the promoters from Bacillus licheniformis alpha-amylase gene (amyL), Bacillus amyloliquefaciens alpha-amylase gene (amyQ), and the Bacillus thuringiensis cryIIIA promoter including stabilizing sequence.

For each of the lactase expression constructs, the SOE-PCR product was transformed into Bacillus subtilis and integrated in the chromosome by homologous recombination into the pectate lyase locus. Subsequently a recombinant Bacillus subtilis clone containing the integrated expression construct was grown in liquid culture. The culture broth was centrifuged (20,000×g, 20 min) and the supernatant was carefully decanted from the precipitate and used for purification of the enzyme or, alternatively, sterile filtered supernatant was used directly for assays.

### Purification of the Recombinant Enzyme by Immobilized Metal Affinity Chromatography

The pH of the cleared supernatant was adjusted to pH 8, filtrated through a 0.2 μM filter, and the supernatant was applied to a 5 ml HisTrap™ Excel column. Prior to loading, the column had been equilibrated in 5 column volumes (CV) of 50 mM Tris/HCl PH 8. To remove unbound material, the column was washed with 8 CV of 50 mM Tris/HCl PH 8, and elution of the target was obtained with 50 mM HEPES pH 7+10 mM imidazole. The eluted protein was desalted on a HiPrep™ 26/10 desalting column, equilibrated using 3 CV of 50 mM HEPES pH 7+100 mM NaCl. This buffer was also used for elution of the target with a flow rate of 10 ml/min. Relevant fractions were selected and pooled based on the chromatogram and SDS-PAGE analysis.

### UHT Treatment

The enzymes of Table 5 were added to skimmed milk and a UHT treatment was performed followed by incubation at 23° C. for 0.5 hrs and for 72 hrs with measurement of lactase activity and lactose being performed after each of these two intervals as described below.

Between 5.5 and 31.6 mg enzyme protein (ep) per litre skimmed milk was used depending on the enzyme. For most of the enzymes 12.7 mg ep/L skimmed milk was used (see Table 7, column 2). Sodium azide was added to a final concentration of 0.025% (w/v) in all milk tested to avoid microbial growth since due to handling etc. the tubes are not fully sterile despite the UHT step. The milk samples were applied to a lab. scale UHT setup as described below. A syringe with a 10 ml milk sample was connected to a long Teflon tube (internal diameter of 0.8 mm) that was immersed sequentially in four baths. The first bath was in silicone at 90° C. with 3 m of the Teflon tube, the second connected bath was in silicone at 140° C. with 50 cm of the Teflon tube, the third silicone bath was at 70° C. with 3 m of Teflon tube, and the final bath was an ice/water bath (0° C.) with 1 m of Teflon tube to cool the milk. A flow of 3 ml/min was applied to the syringe ensuring that the milk samples were incubated at 90° C. for 30 sec, 140° C. for 5 sec and 70° C. for 30 sec before cooling for 10 sec in the ice/water bath, thereby cooling the milk to a temperature in the range of 0-10° C. Finally, the milk was collected in tubes after the ice/water bath, and the samples were incubated at 23° C. for 0.5 h and 72 h, respectively, and then assayed for residual activity and lactose amount using High-Performance Anion-Exchange Chromatography with Pulsed Amperometric Detection (HPAEC-PAD).

### Residual Activity Assay

Samples were centrifuged at 20,600 g in a precooled centrifuge for 45 min at 5° C. and the supernatant was diluted with 20 mM sodium succinic acid and 0.01% Triton X-100, pH 6.5, so an absorbance reading below 1.5 at 405 nm could be measured. Twenty-five μl of each sample was mixed with 175 μl of ONPG substrate (1.67 mg/ml ONPG (o-NitroPhenyl β-D-galatopyranoside, ˜5.5 mM), 0.05 M MES, 1 mM MgSO4, 150 mM KCl, 0.01% Triton X-100, pH 6.5) and incubated for 2.5 hr at 40° C. and stopped by adding 50 μl Na2CO3+5 mM Na4EDTA and measured at 405 nm. Residual activity in % was calculated using the formulae=((Abs405^{heat-treated sample}−Abs405^{blank})*dilution factor)/((Abs405^{untreated sample}−Abs405^{blank})*dilution factor)*100%.

The heat-treated sample is enzyme mixed with skimmed milk and sodium azide and subjected to UHT treatment and subsequent cooling as described above followed by incubation at 23° C. for 0.5 h and 72 h. The untreated sample is the same enzyme mixed with skimmed milk and sodium azide but without UHT treatment and incubation. Both of these samples were diluted to obtain an absorption at 405 nm in the range of 0.5-1.0. The blank is sample without enzyme and the same dilution factor has been used as for the sample with enzyme. The results are shown in Table 8 below.

### Analysis of Residual Lactose Content

The analysis of residual lactose was performed by high-performance anion exchange chromatography coupled with pulsed amperometry detector (HPAEC-PAD).

### Sample Preparation for HPAEC-PAD:

The enzyme was inactivated by adding 5 ul glacial acetic acid to 1 ml milk sample and heated to 90° C. for 5 min and centrifugated at 20,000 g for 10 min. 50 ul sample was transferred to 5 ml Eppendorf tube containing 500 ul MQ (Milli-Q) water. 10 ul Carrez I solution was added and mixed, and then 10 ul Carrez II solution was added and mixed. Then, 4.43 ml MQ water (total volume of 5 ml) was added and mixed. Centrifugation was carried out at 20,000 g for 5 min. The supernatant was diluted ×5 with Milli Q water. These samples were analyzed on HPAEC-PAD.

### Lactose Determination Using HPAEC-PAD

The analysis performed is essentially as described in Leeuwen S, Kuipers B, Dijkhuizen L, Kamerling J. Comparative structural characterization of 7 commercial galacto-oligosaccharide (GOS) products, Carbohydrate Research, 425 (2016) 48-58 with minor modifications, e.g., with a shorter gradient as specified below. Dionex ICS-6000 workstation (Dionex, Amsterdam, The Netherlands) was used, equipped with a CarboPac PA1 4×50 mm Guard Column (Dionex, product no. 043096) followed by CarboPac PA1 4×250 mm (Dionex, product no. 035391) and an ICS-6000 DC ECD detector (Dionex), using a complex gradient of A: Milli-Q water, B: 600 mM NaOAc in 100 mM NaOH, C: 100 mM NaOH, and D: 50 mM NaOAc. The fractionations were performed at 1.0 mL/min with 85% A, 0% B, 10% C, and 5% D in 25 min linear gradient to 10% A, 0% B, 40% C, and 50% D, followed by a 2-min linear gradient to 0% A, 25% B, 75% C and 0% D, directly followed by 5 min washing with 100% B and reconditioning for 15 min with 85% A, 0% B, 10% C, and 5% D. A lactose standard was used to determine the amount of lactose in the enzyme treated samples. The results are shown in Table 7 below, columns 3 and 4.

Dose-Response Curve for Non-UHT Treated B. bifidum Lactase

To be able to compare the activities of the enzymes in the UHT-treated samples to the activity of non-UHT treated B. bifidum lactase (enzyme #31, SEQ ID NO: 1), a dose/response curve for the non-UHT treated B. bifidum lactase (enzyme #31, SEQ ID NO: 1) was made.

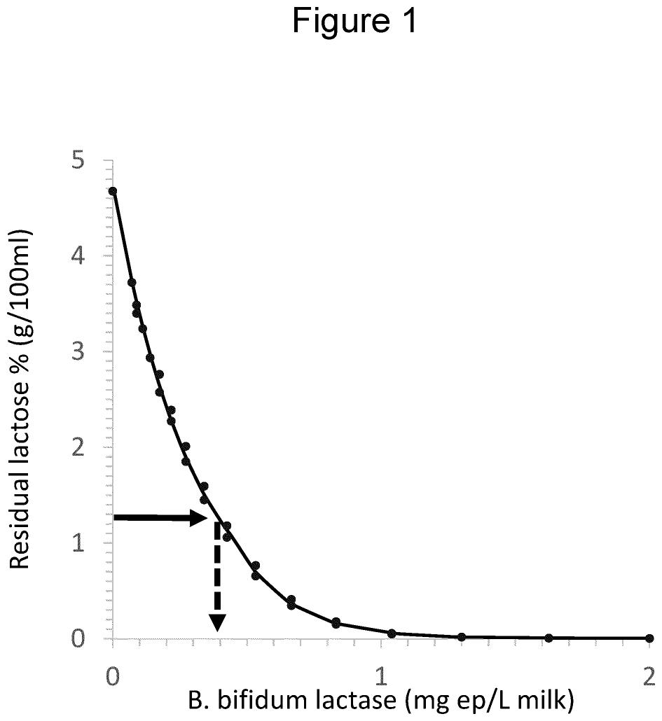

Following doses of B. bifidum lactase (#31) in skim milk (+0.025% sodium azide) were made in duplicate: 2.03, 1.63, 1.30, 1.04, 0.832, 0.666, 0.532, 0.426, 0.341, 0.273, 0.218, 0.174, 0.140, 0.112, 0.0893, 0.0715 mg ep/litre skim milk and the milk with enzyme was incubated for 3 days at room temperature (23° C.). The residual lactose in each sample was determined as described in the above paragraph “Analysis of residual lactose content” using HPAEC-PAD. The dose/response curve was generated by plotting the measured lactose levels against the dosage of the 30 B. bifidum lactase (#31). See FIG. 1.

The curve was used to determine the relative activity of each UHT-treated lactase in % of the activity of non-UHT treated B. bifidum lactase (#31). The data of columns 2 and 4 of Table 7 were used. For each lactase, the value of column 4 (residual lactose (%) after 72 hrs incubation) was with the help of the curve used to determine the “corresponding amount” of non-UHT treated B. bifidum lactase (mg ep/L milk) needed to get to the same residual lactose (%) after 72 hrs incubation at the same temperature. The relative activity of the UHT-treated lactase in % of the activity of non-UHT treated B. bifidum lactase was calculated as the reciprocal of the amount (mg ep/L milk) of UHT-treated lactase (column 2 of Table 7) in percent of the reciprocal of the “corresponding amount” (mg ep/L milk) of non-UHT treated B. bifidum lactase, i.e. the amount needed to get to the same residual lactose content after incubation at 23° C. for 72 hrs, said value being determined with the help of the dose/response curve. In practice, the calculation can be simplified by dividing the “corresponding amount” (mg ep/L milk) of non-UHT treated B. bifidum lactase by the amount (mg ep/L milk) of UHT treated lactase found in column 2 of Table 7 and expressing the result in percent. The thus determined relative activity for each lactase is shown in column 6 of Table 7.

This value is thus a measure of the “survival of enzyme activity after UHT treatment” based on the remaining lactose after UHT treatment and 3 days incubation at 23° C. The dose/response curve for non-UHT treated B. bifidum lactase (SEQ ID NO: 1) (which has also been incubated for 3 days at 23° C.) is used to determine the “corresponding amount” of non-UHT treated B. bifidum lactase.

TABLE 5
Overview of lactase enzymes used in this example. All PE variants
are variants of SEQ ID NO: 1, 4, 5 or 11 as indicated

| Enzyme | SEQ | | WT/PE variant with aa | | |
|---|---|---|---|---|---|
| # | ID NO | Donor | substitutions as indicated | CBM | Length |
| 1 | | Bifidobacterium | C372A | CBM32 | 1298 |
| | | samirii | | | |
| 2 | | Bifidobacterium | A1073C H1122C | CBM32 | 1298 |
| | | samirii | | | |
| 3 | | Urmitella | P620T | CBM32 | 1307 |
| | | timonensis | | | |
| 4 | | Streptococcus | L1064C Y1110C | CBM32 | 1288 |
| | | entericus | | | |
| | | DSM 14446 | | | |
| 5 | | Urmitella | G386Q | CBM32 | 1307 |
| | | timonensis | | | |
| 6 | | Bifidobacterium | P615T | CBM32 | 1298 |
| | | samirii | | | |
| 7 | | Bifidobacterium | P65A | CBM32 | 1298 |
| | | samirii | | | |
| 8 | | Bifidobacterium | C1195G | CBM32 | 1298 |
| | | samirii | | | |
| 9 | SEQ ID | Bifidobacterium | Wild-type | CBM32 | 1298 |
| | NO: 4 | samirii | | | |
| 10 | | Streptococcus | P52A | CBM32 | 1288 |
| | | entericus | | | |
| | | DSM 14446 | | | |
| 11 | SEQ ID | Urmitella | Wild-type | CBM32 | 1307 |
| | NO: 5 | timonensis | | | |
| 12 | | Bifidobacterium | P65A C372A R389A G482A | CBM32 | 1304 |
| | | bifidum | T523N T756K A936S | | |
| | | | T972C I1035C A1129C | | |
| | | | F1132C K1168C S1195C | | |
| 13 | | Streptococcus | G607T | CBM32 | 1288 |
| | | entericus | | | |
| | | DSM 14446 | | | |
| 14 | | Bifidobacterium | P65A C372A R389A G482A | CBM32 | 1304 |
| | | bifidum | T523N P615T T756K | | |
| | | | A936S T972C I1035C | | |
| | | | A1129C F1132C K1168C | | |
| | | | S1195C | | |
| 15 | | Bifidobacterium | P65A C372A M386Q | CBM32 | 1304 |
| | | bifidum | R389A G482A T523N | | |
| | | | T756K A936S T972C | | |
| | | | I1035C A1129C F1132C | | |
| | | | K1168C S1195C | | |
| 16 | SEQ ID | Varibaculum sp. | Wild-type | CBM32 | 1309 |
| | NO: 6 | | | | |
| 17 | | Bifidobacterium | C372A M386Q R389A | CBM32 | 1304 |
| | | bifidum | G482A T523N T756K | | |
| | | | A936S T972C I1035C | | |
| | | | A1129C F1132C K1168C | | |
| | | | S1195C | | |
| 18 | | Bifidobacterium | C372A M386Q R389A | CBM32 | 1304 |
| | | bifidum | G482A T523N P615T | | |
| | | | T756K A936S T972C | | |
| | | | I1035C A1129C F1132C | | |
| | | | K1168C S1195C | | |
| 19 | | Bifidobacterium | C372A R389A G482A | CBM32 | 1304 |
| | | bifidum | T523N T756K A936S | | |
| | | | T972C I1035C A1129C | | |
| | | | F1132C K1168C S1195C | | |
| 20 | | Bifidobacterium | C372A | CBM32 | 1304 |
| | | bifidum | | | |
| 21 | SEQ ID | Clostridium | Wild-type | CBM71 | 1291 |
| | NO: 7 | nexile | | | |
| | | CAG: 348 | | | |
| 22 | SEQ ID | Bacillus sp. S3 | Wild-type | CBM71 | 1313 |
| | NO: 8 | | | | |
| 23 | | Bifidobacterium | C372A R389A G482A | CBM32 | 1304 |
| | | bifidum | T523N P615T T756K | | |
| | | | A936S T972C I1035C | | |
| | | | A1129C F1132C K1168C | | |
| | | | S1195C | | |
| 24 | SEQ ID | Neobacillus | Wild-type | CBM71 | 1130 |
| | NO: 9 | bataviensis | | | |
| 25 | | Bifidobacterium | C372A R389A G482A | CBM32 | 1304 |
| | | bifidum | T523N T756K A936S | | |
| | | | T972C I1035C A1129C | | |
| | | | F1132C K1168C S1195C | | |
| | | | C1199S | | |
| 26 | | Bifidobacterium | N275S C372A R389A G482A | CBM32 | 1304 |
| | | bifidum | T523N T756K A936S | | |
| | | | T972C I1035C T1076C | | |
| | | | Y1122C A1129C S1195C | | |
| | | | C1199S | | |
| 27 | | Bifidobacterium | P65A C372A M386Q | CBM32 | 1304 |
| | | bifidum | R389A G482A T523N | | |
| | | | P615T T756K A936S | | |
| | | | T972C I1035C A1129C | | |
| | | | F1132C K1168C S1195C | | |
| 28 | SEQ ID | Neobacillus | Wild-type | CBM71 | 1313 |
| | NO: 10 | mesonae | | | |
| 29 | | Bifidobacterium | C372A R389A G482A T523N | CBM32 | 1304 |
| | | bifidum | T756K A936S T972C | | |
| | | | I1035C A1129C S1195C | | |
| 30 | SEQ ID | Streptococcus | Wild-type | CBM32 | 1288 |
| | NO: 11 | entericus | | | |
| | | DSM 14446 | | | |
| 31 | SEQ ID | Bifidobacterium | Wild-type | CBM32 | 1304 |
| | NO: 1 | bifidum | | | |
| 32 | SEQ ID | Streptomyces | Wild-type | CBM71 | 1678 |
| | NO: 12 | cirratus | | | |
| 33 | SEQ ID | Bifidobacterium | Wild-type | CBM32 | 1299 |
| | NO: 13 | aerophilum | | | |
| 34 | SEQ ID | Neobacillus | Wild-type | CBM71 | 1309 |
| | NO: 14 | niacini | | | |
| 35 | SEQ ID | Bifidobacterium | Wild-type | CBM32 | 1304 |
| | NO: 15 | mongoliense | | | |

TABLE 6
Matrix of sequence identities between the C-terminally
truncated wild-type sequences of Table 5

| SEQ | | | | | | | | | | | | | |
|---|---|---|---|---|---|---|---|---|---|---|---|---|---|
| ID | | | | | | | | | | | | | |
| NO | 1 | 15 | 4 | 13 | 7 | 5 | 6 | 11 | 12 | 8 | 9 | 10 | 14 |
| 1 | 100 | 98 | 70 | 75 | 57 | 51 | 51 | 47 | 39 | 43 | 44 | 43 | 43 |
| 15 | 98 | 100 | 70 | 75 | 57 | 51 | 50 | 47 | 39 | 43 | 44 | 43 | 43 |
| 4 | 70 | 70 | 100 | 69 | 54 | 51 | 51 | 48 | 40 | 44 | 45 | 43 | 44 |
| 13 | 75 | 75 | 69 | 100 | 56 | 50 | 50 | 47 | 40 | 43 | 44 | 43 | 43 |
| 7 | 57 | 57 | 54 | 56 | 100 | 51 | 51 | 47 | 39 | 44 | 44 | 42 | 44 |
| 5 | 51 | 51 | 51 | 50 | 51 | 100 | 98 | 44 | 39 | 40 | 40 | 40 | 39 |
| 6 | 51 | 50 | 51 | 49 | 51 | 98 | 100 | 43 | 37 | 40 | 41 | 40 | 39 |
| 11 | 46 | 46 | 47 | 47 | 47 | 44 | 43 | 100 | 38 | 41 | 42 | 41 | 41 |
| 12 | 40 | 40 | 40 | 40 | 38 | 38 | 37 | 38 | 100 | 48 | 49 | 48 | 46 |
| 8 | 43 | 43 | 44 | 43 | 44 | 39 | 40 | 41 | 48 | 100 | 93 | 85 | 89 |
| 9 | 43 | 43 | 44 | 43 | 44 | 40 | 41 | 42 | 48 | 93 | 100 | 88 | 91 |
| 10 | 43 | 43 | 43 | 43 | 42 | 39 | 40 | 41 | 47 | 84 | 87 | 100 | 86 |
| 14 | 43 | 43 | 44 | 43 | 44 | 38 | 39 | 40 | 46 | 89 | 91 | 86 | 100 |

TABLE 7
Lactase was added to skimmed milk which was UHT treated followed by 30 minutes and
72 hours incubation at 23° C. The residual lactose in the milk was determined
and used to calculate the relative activity of the enzyme after UHT treatment in percent
of the activity of non-UHT treated B. bifidum lactase (enzyme #31) as well
as the extrapolated amount of enzyme (mg ep/L milk) needed to get to 1.41%, 0.1%
and 0.01% residual lactose after UHT treatment and incubation at 23° C. for 72 hours

| | | | | | | (mg ep/L | (mg ep/L | (mg ep/L |
|---|---|---|---|---|---|---|---|---|
| | | residual | residual | | Relative | milk) to | milk) to | milk) to |
| | | lactose | lactose | gram | activity to | reach | reach | reach |
| | mg | (%) | (%) | lactose/mg | unstressed | 1.41% | 0.1% | 0.01% |
| Enz | ep/L | after | after | ep (from | B. bifidum | residual | residual | residual |
| # | milk | 0.5 hr | 72 hr | 0.5 to 72 h) | lactase (%) | lactose | lactose | lactose |
| 1 | 10.6 | 4.4 | 0.007 | 4.2 | 15.9 | 2.3 | 5.9 | 9.8 |
| 2 | 9.0 | 4.6 | 0.020 | 5.0 | 13.7 | 2.6 | 6.8 | 11.4 |
| 3 | 12.3 | 4.5 | 0.009 | 3.6 | 11.6 | 3.1 | 8.0 | 13.4 |
| 4 | 9.4 | 4.2 | 0.059 | 4.5 | 11.1 | 3.2 | 8.4 | 14.0 |
| 5 | 16.2 | 4.2 | 0.006 | 2.6 | 10.9 | 3.3 | 8.6 | 14.3 |
| 6 | 12.9 | 4.6 | 0.019 | 3.5 | 9.9 | 3.7 | 9.5 | 15.8 |
| 7 | 13.1 | 4.6 | 0.020 | 3.5 | 9.5 | 3.8 | 9.8 | 16.4 |
| 8 | 6.4 | 4.6 | 0.54 | 6.3 | 9.2 | 3.9 | 10.1 | 16.9 |
| 9 | 13.4 | 4.4 | 0.042 | 3.3 | 8.3 | 4.3 | 11.3 | 18.8 |
| 10 | 11.9 | 4.4 | 0.092 | 3.6 | 8.0 | 4.5 | 11.7 | 19.6 |
| 11 | 5.5 | 4.5 | 1.27 | 5.9 | 7.1 | 5.1 | 13.1 | 22.0 |
| 12 | 12.7 | 4.6 | 0.36 | 3.3 | 5.3 | 6.9 | 17.8 | 29.7 |
| 13 | 17.4 | 4.3 | 0.20 | 2.4 | 4.5 | 8.0 | 20.7 | 34.6 |
| 14 | 12.7 | 4.6 | 0.71 | 3.1 | 4.2 | 8.6 | 22.3 | 37.3 |
| 15 | 12.7 | 4.6 | 0.85 | 3.0 | 3.9 | 9.3 | 24.0 | 40.2 |
| 16 | 12.7 | 4.5 | 0.86 | 2.9 | 3.9 | 9.4 | 24.2 | 40.5 |
| 17 | 12.7 | 4.6 | 0.95 | 2.9 | 3.7 | 9.7 | 25.2 | 42.1 |
| 18 | 12.7 | 4.6 | 1.03 | 2.8 | 3.6 | 10.1 | 26.1 | 43.7 |
| 19 | 12.7 | 4.6 | 1.24 | 2.6 | 3.2 | 11.5 | 29.6 | 49.6 |
| 20 | 12.8 | 4.6 | 1.42 | 2.4 | 2.8 | 12.9 | 33.5 | 56.1 |
| 21 | 6.6 | 4.6 | 2.68 | 2.9 | 2.6 | 14.1 | 36.5 | 61.0 |
| 22 | 22.2 | 4.0 | 0.60 | 1.5 | 2.5 | 14.2 | 36.8 | 61.5 |
| 23 | 12.7 | 4.6 | 1.60 | 2.4 | 2.5 | 14.2 | 36.8 | 61.5 |
| 24 | 12.4 | 4.5 | 1.66 | 2.3 | 2.5 | 14.3 | 37.1 | 62.0 |
| 25 | 12.7 | 4.6 | 1.70 | 2.3 | 2.4 | 15.0 | 38.9 | 65.0 |
| 26 | 12.7 | 4.6 | 1.71 | 2.3 | 2.4 | 15.1 | 39.0 | 65.3 |
| 27 | 12.7 | 4.6 | 1.75 | 2.3 | 2.3 | 15.4 | 40.0 | 66.9 |
| 28 | 11.2 | 4.5 | 2.14 | 2.1 | 2.1 | 17.1 | 44.2 | 73.9 |
| 29 | 12.7 | 4.6 | 1.97 | 2.1 | 2.1 | 17.5 | 45.4 | 75.9 |
| 30 | 12.7 | 4.6 | 1.99 | 2.1 | 2.0 | 17.7 | 45.8 | 76.6 |
| 31 | 12.7 | 4.6 | 2.22 | 1.9 | 1.8 | 20.3 | 52.5 | 87.8 |
| 32 | 12.7 | 4.6 | 2.38 | 1.7 | 1.6 | 22.4 | 57.9 | 96.8 |
| 33 | 12.7 | 4.6 | 3.19 | 1.1 | 0.9 | 38.6 | 99.9 | 167 |
| 34 | 31.6 | 4.6 | 2.05 | 0.8 | 0.8 | 45.7 | 118 | 197 |
| 35 | 12.8 | 4.6 | 3.49 | 0.9 | 0.7 | 50.2 | 130 | 217 |

TABLE 8
Residual activities in milk after UHT treatment followed
by 30 minutes and 72 hours incubation at 23° C.

| Enzyme | % residual activity | % residual activity | (RA 72 h)/ |
|---|---|---|---|
| # | after 0.5 h | after 72 h | (RA 0.5 h) |
| 1 | 11 | 15 | 1.5 |
| 2 | 2.1 | 13 | 6.2 |
| 3 | 17 | 48 | 2.9 |
| 4 | 13 | 25 | 1.9 |
| 5 | 5.4 | 17 | 3.1 |
| 6 | 2.5 | 8.0 | 3.2 |
| 7 | 1.2 | 5.7 | 4.6 |
| 8 | 3.3 | 11 | 3.2 |
| 9 | 2.1 | 5.1 | 2.4 |
| 10 | 0.8 | 3.3 | 4.2 |
| 11 | 1.3 | 4.2 | 3.2 |
| 12 | 3.0 | 9.9 | 3.3 |
| 13 | 9.7 | 14 | 1.5 |
| 14 | 2.7 | 7.6 | 2.8 |
| 15 | 4.2 | 11.9 | 2.9 |
| 16 | 2.4 | 10.8 | 4.4 |
| 17 | 3.7 | 9.5 | 2.5 |
| 18 | 2.6 | 7.7 | 2.9 |
| 19 | 2.9 | 6.4 | 2.2 |
| 20 | 1.0 | 1.2 | 1.2 |
| 21 | 0.4 | 8.0 | 19.4 |
| 22 | 23 | 34 | 1.5 |
| 23 | 2.5 | 6.3 | 2.5 |
| 24 | 12 | 14 | 1.1 |
| 25 | 4.7 | 8.4 | 1.8 |
| 26 | 2.7 | 7.0 | 2.6 |
| 27 | 3.5 | 8.9 | 2.5 |
| 28 | 12 | 10 | 0.8 |
| 29 | 1.0 | 3.0 | 3.0 |
| 30 | 18 | 37 | 2.0 |
| 31 | 0.2 | 0.7 | 4.3 |
| 32 | 2.1 | 3.5 | 1.7 |
| 33 | 0.2 | 1.8 | 9.1 |
| 34 | 3.3 | 8.1 | 2.5 |
| 35 | 0.1 | 0.8 | 7.4 |

### Results

Table 7 columns 3 and 4 show the residual lactose after UHT treatment followed by incubation at 23° C. for 0.5 hrs and 72 hrs respectively. The reduction in lactose predominantly takes place from 0.5 hrs (an average of 4.5 g lactose/100 ml corresponding to 96% of initial lactose) to 72 hrs, where enzymes #1, 3 and 5 are below 0.01% residual lactose, which is needed to claim lactose free in many countries. The enzyme dose used (mg ep/L milk) is shown for each enzyme in column 2. If the enzyme dose is increased, the residual lactose after 72 hrs will be lower.

Column 5 shows the pseudo-specific activity (gram lactose converted per mg enzyme protein) after UHT treatment calculated as (“g/L lactose at 0.5 h” minus “g/L lactose at 72 h”)/“mg ep/L”, i.e. the value in column 3 minus that in column 4 (although expressed in g/L rather than % lactose) divided with the value in column 2. As an example using the data for enzyme #10 in Table 7: (45 g/L lactose-12.7 g/L lactose)/5.5 mg ep/L=5.9 g lactose/mg ep (per 71.5 hrs). This pseudo-specific activity is biased in the way that it is dose dependent, since the residual lactose versus enzyme dose is not linear (see FIG. 1). As a result, a relatively low enzyme dose gives a higher pseudo-specific activity value compared to a higher enzyme dose.

To have a better comparison of the efficiency of the enzymes, a “Relative activity to unstressed B. bifidum lactase (#31)” has been calculated as described above and is shown in column 6. An example of a calculation for enzyme #10 is shown in FIG. 1: According to Table 7, a dose of 5.5 mg ep/litre milk of enzyme #10 is used resulting in 1.27% residual lactose after 72 hrs (see horizontal arrow in FIG. 1). The “corresponding amount” of unstressed (non-UHT treated) B. bifidum lactase (enzyme #31) would be 0.39 mg ep (vertical arrow in FIG. 1). This provides a relative activity of 7.1% (0.39/5.5*100%) as can be seen for enzyme #10 in Table 7, column 6. This is a very good measure of the relative activity of the enzyme after UHT treatment compared to the non-UHT treated B. bifidum lactase (#31) determined as the reciprocal of the amount of ep needed to get to a certain residual lactose content after UHT treatment and incubation at 23° C. for 72 hrs in percent of the reciprocal of the “corresponding amount” of ep of non-UHT treated B. bifidum lactase needed to get to the same residual lactose content after incubation at 23° C. for 72 hrs.

Columns 7, 8 and 9 show the calculated enzyme doses needed to reach after storage at 23° C. for 72 hrs 1.41% remaining lactose (stomach friendly level can be 70% reduction of lactose), 0.1% remaining lactose (to claim low lactose level in many countries) or 0.01% remaining lactose (to claim lactose free level in many countries). These numbers are calculated as follows: The mg ep/L of B. bifidum lactase (enzyme #31) to reach lactose levels of 1.41%, 0.1% and 0.01% is 0.361, 0.934 and 1.56 mg ep/L milk, respectively (determined from the dose-response curve in FIG. 1). The “Relative activity to unstressed B. bifidum lactase (%)” number (column 6 in table 7) is used to predict the corresponding number for each enzyme, e.g. for enzyme #10: the “Relative activity to unstressed B. bifidum lactase (%)” number is 7.1% (0.071) and therefore the corresponding amount of enzyme protein for enzyme #10 is 0.361/0.071, 0.934/0.071 and 1.56/0.071 mg ep/L milk which is 5.1, 13.1 and 22.0 mg ep/L milk.

Enzyme doses of 5-50 mg lactase ep/L milk (4.5-5.0% lactose) is typically used in industrial batch lactose-reducing application with a tank incubation in the range of 8-24 hrs at 4-10° C. to reach lactose levels of 0.01-0.1% lactose (%=g lactose/100 ml milk). Thus, the top candidates in Table 7 are highly relevant for lactose-free applications (0.01% lactose). whereas other sequences with a lower relative activity may be more relevant for low-lactose applications rather than lactose-free.

Table 8, columns 2 and 3, show residual activity of the enzymes at 0.5 hrs and 72 hrs, respectively, after UHT treatment. There are various degrees of residual activity and most of the enzymes surprisingly have an increased residual activity after 72 hrs compared to 0.5 hrs with an average of more than 3-fold for all listed enzymes.

In Example 2, the residual activity of the lactase of SEQ ID NO: 1 was determined as 2.14% after incubation at 70° C. for 30 sec and 140° C. for 5 sec before cooling. In this example, the lactases, including the lactase of SEQ ID NO: 1 (enzyme #31), were incubated at 90° C. for 30 sec, 140° C. for 5 sec and 70° C. for 30 sec before cooling and therefore the residual activity of the lactase of SEQ ID NO: 1 was lower.

The reason for the relatively low correlation between % residual activity (Table 8) and measured residual lactose levels in Table 7 is believed to be the different specific activities for lactose of each enzyme. The ability of an enzyme to hydrolyse lactose after UHT treatment is influenced by the enzyme's efficiency to convert lactose (specific activity before UHT treatment) as well as the enzyme's ability to be active after UHT treatment (% residual activity).

Amino acid changes have been made which have improved the performance of the enzymes. Especially a replacement of a free Cys (homologous to C372 in enzyme #31), e.g. in enzyme #1 (Bifidobacterium samirii, C372A), an introduction of a disulfide, e.g. in enzymes #2 and 4, and replacement of cis-prolines, e.g. in enzyme #s 3, 6 and 7.

This example shows that all of the enzymes in Table 5 are suitable for use in the methods as claimed herein.

All of the enzymes in Table 5 are bacterial GH2 lactases of clade DYLGE. They all comprise the motifs WTXXDY[I/L/R]GE[P/S/A], SR[W/Y/F]YSGSGX[Y/G]R and [L/V/I]X[L/V/I]PHD.

### Note Regarding FIG. 1:

Table 9 below in the first two columns show the data points of FIG. 1.

TABLE 9

| mg | Lactose % | |
|---|---|---|
| ep/L | (g/100 ml | calculated mg |
| milk | milk) | ep/L |
| 0 | 4.67 | |
| 0 | 4.67 | |
| 0.071 | 3.72 | 0.073 |
| 0.089 | 3.49 | 0.092 |
| 0.089 | 3.40 | 0.10 |
| 0.11 | 3.24 | 0.11 |
| 0.14 | 2.94 | 0.14 |
| 0.17 | 2.57 | 0.18 |
| 0.17 | 2.76 | 0.16 |
| 0.22 | 2.27 | 0.22 |
| 0.22 | 2.39 | 0.20 |
| 0.27 | 1.85 | 0.28 |
| 0.27 | 2.01 | 0.26 |
| 0.34 | 1.45 | 0.35 |
| 0.34 | 1.59 | 0.32 |
| 0.43 | 1.06 | 0.45 |
| 0.43 | 1.18 | 0.41 |
| 0.53 | 0.657 | 0.55 |
| 0.53 | 0.766 | 0.51 |
| 0.67 | 0.348 | 0.68 |
| 0.67 | 0.412 | 0.64 |
| 0.83 | 0.152 | 0.85 |
| 0.83 | 0.180 | 0.81 |
| 1.04 | 0.049 | 1.08 |
| 1.04 | 0.054 | 1.06 |
| 1.04 | 0.057 | 1.05 |
| 1.30 | 0.019 | 1.28 |
| 1.30 | 0.019 | 1.28 |
| 1.63 | 0.0083 | 1.65 |
| 1.63 | 0.0081 | 1.66 |
| 1.63 | 0.0083 | 1.65 |
| 2.03 | 0.0037 | 2.01 |
| 2.03 | 0.0038 | 2.00 |

Instead of manual/visual reading the curve, a fitting equation was made using equation 1 below, and the “a” and “b” numbers in Table 10 with the % lactase mentioned in the headings. The calculated mg ep/L obtained using the Lactose % in Table 9 is shown in column 3 of Table 9.

mg ep/L milk=a*Ln(lactose %)+b Equation 1:

TABLE 10

| | 1.0-4.7% | 0.1-1.0% | 0.003-0.1% |
|---|---|---|---|
| | lactose | lactose | lactose |
| a | −0.297 | −0.206 | −0.452 |
| b | 0.4631 | 0.4601 | −0.5183 |

### Example 5

### Thermal Shift Assay at pH 6 and 7

The thermal shift assay (TSA) measures the melting temperature of a protein (Tm), which is the temperature at which there is 50% denaturation. Protein denaturation is monitored via an increase in fluorescence of SYPRO Orange dye which binds to hydrophobic residues that get exposed as the target protein unfolds.

The purified samples were diluted to 0.24 mg/ml in MilliQ water. The Thermal shift assay mix was prepared by diluting SyProOrange (Invitrogen/ThermoFisher #S6650) 200-fold into the desired pH buffer (100 mM succinic acid, 100 mM HEPES, 100 mM glycine, 150 mM KCl, 1 mM CaCl2, 0.01% TritonX100, adjusted to pH 6 and 7). 10 μl of diluted sample was added to 20 μl TSA mix in a 96-well (Light Cycler 480 multi-well plate, Roche #04729692001). After sealing with optical tape (Roche, #04729757001), the plate was heated up from 25° C. to 95° C. (temperature ramp: 3.2° C./minute) in a LightCycler 480 II Real-Time PCR machine (Roche) and the fluorescence continuously measured (Excision/emission wavelengths: 465/510 nm). Tm is identified by plotting the first derivative of the fluorescence as a function of temperature (dF/dT) and determining the temperature with maximal dF/dt.

TABLE 11
Melting temperature Tm (° C.) as determined by TSA. The
enzyme numbers (enzyme #) are according to Table 5

| Enzyme # | Donor | PE | Tm @ pH 6 | Tm @ pH 7 |
|---|---|---|---|---|
| 9 | Bifidobacterium | | 56.2 | 57.2 |
| | samirii | | | |
| 11 | Urmitella | | 53.4 | 55.0 |
| | timonensis | | | |
| 30 | Streptococcus | | 53.0 | 54.1 |
| | entericus | | | |
| 31 | Bifidobacterium | | 55.8 | 56.6 |
| | bifidum | | | |
| 32 | Streptomyces | | 55.5 | 55.8 |
| | cirratus | | | |
| 22 | Bacillus | | 66.6 | 66.7 |
| | sp. S3 | | | |
| 1 | Bifidobacterium | C372A | 56.4 | 57.0 |
| | samirii | | | |
| 2 | Bifidobacterium | A1073C | 57.0 | 57.0 |
| | samirii | H1122C | | |
| 3 | Urmitella | P620T | 50.0 | 50.6 |
| | timonensis | | | |
| 5 | Urmitella | G386Q | 50.9 | 50.6 |
| | timonensis | | | |
| 16 | Varibaculum sp. | | 62.6 | 63.1 |
| 4 | Streptococcus | L1064C | 52.2 | 53.9 |
| | entericus | Y1110C | | |
| 8 | Bifidobacterium | C1195G | 57.7 | 58.1 |
| | samirii | | | |

### Example 6

### Temperature Profiles

### Assay for Determining Temperature Profile, 35° C.-75° C.:

Temperature profile to determine the temperature optimum is prepared by adding 10 μl diluted enzyme samples (diluted with 50 mM succinate, 50 mM HEPES, 50 mM CHES, 150 mM KCl, 2 mM CaCl2, 1 mM MgCl2+0.01% triton X-100, pH 6.5) to PCR tubes. Then 90 μl substrate (167 mM lactose, 50 mM succinate, 50 mM HEPES, 50 mM CHES, 150 mM KCl, 2 mM CaCl2, 1 mM MgCl2, pH 6.5) is added and the PCR tubes is placed in a preheated PCR block with temperature gradient 35-75° C. (using TProfessional thermocycler, Biometra) and incubated for 30 min at 35-75° C. (gradient), and then placed on ice. The reaction is stopped by adding 100 μl 0.25 M NaOH. Twenty μl is transferred to a 96 well microtiter plate, and 230 μL GOD-Perid (100 mM potassium phosphate buffer, pH 7, 0.6 g/l Glucose oxidase, 0.02 g/l horseradish peroxidase, 1.0 g/l ABTS) solution is added. After 30 minutes in the dark at room temperature the absorbance is measured at 420 nm. The initial dilution of the enzyme should be adjusted so the final 420 nm reading at the optimum temperature is between abs. 0.5-2.5. The temperature with the highest delta Abs at 420 nm (“Abs 420 nm with enzyme” minus “Abs 420 nm without enzyme”, i.e. background) is set to 100% (temperature optimum), and relative activity at other temperatures based on delta Abs 420 nm in relation to the delta Abs 420 nm with the highest value is used to determine a temperature profile.

The assay was performed using the lactase enzymes as indicated in Table 12 below. The enzyme numbers (enzyme #) are according to Table 5.

TABLE 12
Temperature profiles. Relative activity in percent
of the activity at the optimum temperature

| Temp. | enzyme | enzyme | enzyme | enzyme | enzyme |
|---|---|---|---|---|---|
| (° C.) | # 9 | # 11 | # 30 | # 31 | # 16 |
| 35 | 89 | 92 | 92 | 99 | 100 |
| 36.1 | 93 | 92 | 98 | 91 | 98 |
| 38.9 | 96 | 98 | 100 | 97 | 93 |
| 43 | 100 | 97 | 97* | 100 | 71 |
| 47.5 | 95 | 100 | 28* | 99 | 57* |
| 52.5 | 77* | 79* | 17 | 93 | 48* |
| 57.5 | 31* | 29* | 8 | 68* | 29 |
| 62.3 | 6 | 8 | 4 | 19* | 14 |
| 67 | 4 | 5 | 4 | 6 | 7 |
| 71.1 | 3 | 4 | 4 | 5 | 4 |
| 73.9 | 3 | 3 | 4 | 5 | 3 |
| 75 | 2 | 3 | 3 | 4 | 4 |

The temperature profile from 35-75° C. can be seen in Table 12 and shows a temperature optimum close to 43.0° C., 47.5° C., 38.9° C., 43° C. and <35° C. for enzyme #9, 11, 30, 31 and 16, respectively. The temperature where 50% activity remains (after temperature optimum) is approximately 55° C., 55° C., 46° C., 58° C. and 52° C., respectively (evaluated from Table 12, in-between the two numbers with an asterisk *). If unfolding of the enzyme is fully reversible then the decline in activity after optimum should corelate to the amount of unfolded enzyme and the “melting temperature” (Tm, where half of the enzyme is unfolded) seen in Table 11 should be close to 50% remaining activity obtained from this Table 12. However, typically the unfolding is irreversible and there is also a temperature dependence of the lactose hydrolysis rate which both have an impact on the decline in activity. As Tm determined in Example 5 is approximately 57° C., 55° C., 54° C., 56° C. and 63° C., respectively for enzyme #9, 11, 30, 31 and 16 (see Table 11), there is a close match between Tm and 50% remaining activity for enzyme #9, 11 and 31 which suggest that these enzymes have pronounced amount of refolding in the assay.

The minor amount of relative activity (2-5%) at higher temperature (>70° C.) which seems constant is probably due to lactose hydrolysis during the ramp up from room temp to >70° C. (ramp up time is expected to be below 1 min).

### Example 7

### Reducing Agent as Scavenger for Oxidation of Free Cysteines

There are two free cysteines (i.e. not in a disulfide form) in Enzyme #31 that potentially can be oxidized (C372 and C1199) and hereby prevent refolding. This experiment was made to see if the reducing agent dithiothreitol (DTT) could be a scavenger for this Cys oxidation by reacting with the oxidative compounds present in milk. The UHT treatment and measurement of the residual activity (RA) was done as described in example 4. As shown in Table 13 below there is a positive effect of adding 0.1 mM DTT, see last column (calculated by “% RA with DTT”/“% RA without DTT”), which suggests that removal of the oxidative compounds present in milk increases the residual activity of the lactase enzyme comprising one or more free cysteines. The 0.1 mM DTT addition has less pronounced effect on the two variants where the cysteines have been substituted with a different amino acid, C372A and C1199S, respectively. When these are compared to the wild-type (Enzyme #31) there is a lower effect of adding the DTT. This is a clear indication that the oxidation of the “free cysteines” in Enzyme #31 affect refolding. The residual activity of the wild-type (Enzyme #31) with 0.1 mM DTT is almost the same as the PE variant C372A, suggesting that C372 is most sensible for oxidation. DTT is incompatible for food consumption, but other reducing agents which are food approved can be added, e.g. L-cysteine, sulphite or glutathione to the milk or to the enzyme formulation.

TABLE 13

| | | | % residual | Improvement |
|---|---|---|---|---|
| Enzyme | aa | | activity | factor of adding |
| # | substitution | additive | after 72 h | 0.1 mM DTT |
| 31 | | no DTT | 0.6 | |
| 31 | | 0.1 mM DTT | 1.2 | 1.9 |
| 20 | C372A | no DTT | 1.2 | |
| 20 | C372A | 0.1 mM DTT | 1.3 | 1.1 |
| | C1199S | no DTT | 0.5 | |
| | C1199S | 0.1 mM DTT | 0.6 | 1.3 |

### Example 8

### Lactase Enzyme of SEQ ID NO: 1 Tested in High Protein Chocolate Milk at Industrial Scale Settings

44 mg ep “SEQ ID NO: 1”/L was added to a high shear mixer with 4000 L high protein chocolate milk containing 4.5% lactose, 8% milk protein and 1% cocoa powder. Elecster 10800 UHT equipment was used (an indirect UHT treatment) with a flow rate of ˜10,000 L/h (90% capacity) and the enzyme was added 30 minutes before UHT-treatment was started. UHT conditions were performed with a protein stabilization at 80° C. for 5 min before high heat step at 140-142° C. for 5 sec (homogenization was done upstream at 70° C., 150/50 bar). Samples were collected and frozen after UHT; one sample was collected after an hour and frozen and the rest of the samples were stored at ambient temperature for 1 to 9 days and then frozen. All samples were analyzed for residual lactose, as described in example 4 using HPAEC-PAD.

TABLE 14

| Incubation | | |
|---|---|---|
| time | Average | Avg. Dev. |
| days | % lactose | % lactose |
| 0.04 | 2.17 | 0.005 |
| 1 | 1.51 | 0.010 |
| 2 | 1.01 | 0.002 |
| 3 | 0.58 | 0.008 |
| 4 | 0.35 | 0.007 |
| 5 | 0.21 | 0.006 |
| 6 | 0.13 | 0.001 |
| 9 | 0.06 | 0.004 |

Table 14 shows the residual lactose after the UHT treatment (data is average of two measurements and Avg. Dev. is “average of the absolute deviations of data points from their mean”). With the dose used, low lactose claim (<0.1% lactose) can be made after 9 days at ambient temperature. If more enzyme had been added, this would have resulted in a correspondingly faster lactose reduction.

### Example 9

### More PE Variants of SEQ ID NO: 4

In this Example, further PE variants of SEQ ID NO. 4 were tested using the same conditions as in Example 4. A sample was included which was incubated at 23° C. for 24 hours after UHT treatment and then assayed for residual activity and lactose amount.

TABLE 15

| | | WT/PE variant with | | |
|---|---|---|---|---|
| Enzyme | | aa substitutions | | |
| # | Donor | as indicated | CBM | Length |
| 36 | Bifidobacterium | C372A A1073C | CBM32 | 1298 |
| | samirii | H1122C | | |
| 37 | Bifidobacterium | C372A C1195G | CBM32 | 1298 |
| | samirii | | | |
| 38 | Bifidobacterium | A1073C H1122C | CBM32 | 1298 |
| | samirii | C1195G | | |
| 39 | Bifidobacterium | C372A A1073C | CBM32 | 1298 |
| | samirii | H1122C C1195G | | |

TABLE 16

| | | | | | | (mg ep/L | (mg ep/L | (mg ep/L |
|---|---|---|---|---|---|---|---|---|
| | | residual | residual | | Relative | milk) to | milk) to | milk) to |
| | | lactose | lactose | gram | activity to | reach | reach | reach |
| | mg | (%) | (%) | lactose/mg | unstressed | 1.41% | 0.1% | 0.01% |
| Enz | ep/L | after | after | ep (from | B. bifidum | residual | residual | residual |
| # | milk | 0.5 hr | 72 hr | 0.5 to 72 h) | lactase (%) | lactose | lactose | lactose |
| 36 | 14.7 | 4.3 | 0.031 | 2.9 | 8.0 | 4.5 | 11.7 | 19.5 |
| 37 | 16.9 | 4.3 | 0.012 | 2.5 | 8.1 | 4.5 | 11.5 | 19.3 |
| 38 | 16.5 | 4.2 | 0.018 | 2.5 | 7.8 | 4.6 | 11.9 | 20.0 |
| 39 | 15.7 | 4.5 | 0.0078 | 2.9 | 10.7 | 3.4 | 8.7 | 14.6 |

TABLE 16A

| | residual |
|---|---|
| | lactose |
| Enz | (%) after |
| # | 24 hr |
| 36 | 0.10 |
| 37 | 0.053 |
| 38 | 0.525 |
| 39 | 0.10 |

TABLE 17

| | % residual | % residual | % residual | |
|---|---|---|---|---|
| Enzyme | activity | activity | activity | (RA 72 h)/ |
| # | after 0.5 h | after 24 h | after 72 h | (RA 0.5 h) |
| 36 | 14.0 | 16.3 | 19.0 | 1.4 |
| 37 | 23.7 | 30.1 | 28.9 | 1.2 |
| 38 | 6.7 | 14.8 | 17.4 | 2.6 |
| 39 | 21.1 | 27.2 | 28.9 | 1.4 |

### Example 10

### Construction of Clades and Phylogenetic Trees

### GH2 Phylogenetic Tree

A phylogenetic tree was constructed, of polypeptide sequences of the invention containing a GH2 domain, as defined in CAZY (Lombard, Henrissat et al, 2014. The carbohydrate-active enzymes database (CAZy) in 2013. Nucleic Acids Res. 42: D490-5, http://www.cazy.org/). The phylogenetic tree was constructed from a multiple alignment of mature polypeptide sequences containing at least one GH2 domain. The sequences were aligned using the MUSCLE algorithm version 3.8.31 (Edgar, 2004. Nucleic Acids Research 32(5): 1792-1797), and the trees were constructed using FastTree version 2.1.8 (Price et al., 2010, PloS one 5(3): e9490) and visualized using iTOL (Letunic & Bork, 2007. Bioinformatics 23(1): 127-128).

A subset of polypeptides containing a GH2 domain, also contains a Glycosyl hydrolase family 2 N terminal domain, as defined by Pfam domain ID PF02837 (The Pfam protein families database: towards a more sustainable future: R. D. Finn, P. Coggill, R. Y. Eberhardt, S. R. Eddy, J. Mistry, A. L. Mitchell, S. C. Potter, M. Punta, M. Qureshi, A. Sangrador-Vegas, G. A. Salazar, J. Tate, A. Bateman, Nucleic Acids Research (2016) Database Issue 44: D279-D285). This domain is involved in binding of galactose. All polypeptides of the invention contain a GH2 domain, as well as this Glycosyl hydrolase family 2 N terminal domain. The Glycosyl hydrolase family 2 N terminal domain will be denoted the GH2N domain. As an example, in SEQ ID NO: 4 from Bifidobacterium samirii, the GH2N domain is located at positions 34 to 178.

### Generation of Clades

In addition to containing a GH2 domain, the polypeptides of the invention also comprise several unique short peptide motifs important for lactase activity. Our data indicate that these motifs are also important for the ability of the enzymes to refold following a thermal treatment such as a UHT treatment.

One example is WTXXDY[I/L/R]GE[P/S/A] (SEQ ID NO: 18) situated in positions corresponding to positions 591 to 600 in Bifidobacterium samirii (SEQ ID NO: 4).

The polypeptides containing a GH2 domain can be separated into distinct sub-clusters. The sub-clusters are defined by one or more short sequence motifs, as well as containing a GH2 domain.

### Generation of the DYLGE Clade

We denoted one sub-cluster comprising the short peptide motif WTXXDY[I/L/R]GE[P/S/A] (SEQ ID NO: 18) as the DYLGE clade. The glutamic acid E in the motif, located at position 599 in SEQ ID NO: 4, is involved in binding of galactose. All polypeptide sequences containing a GH2 domain, as well as the motif will be denoted as belonging to the DYLGE clade. The DYLGE clade may also contain additional short peptide motifs. SR[W/Y/F]YSGSGX[Y/G]R (SEQ ID NO: 19) located at positions 164 to 174 in SEQ ID NO: 4, as well as motif [W/V/I]X[L/V/I]PHD (SEQ ID NO: 20), corresponding to positions 62 to 67 in SEQ ID NO: 4. Both motifs are located in the GH2N galactose binding domain and are important for substrate binding.

### Generation of the MGN Clade

We denoted another sub-cluster of GH2 comprising the motif EYXH[A/S/D/T]MG[N/T/L] (SEQ ID NO: 21), located at positions 532 to 539 in SEQ ID NO: 3, as the MGN clade. The glutamic acid residue E is fully conserved in the clade and act as nucleophile catalyst in the active site of the enzyme ((https://www.uniprot.org/uniprotkb/B3GS90/entry).

All polypeptide sequences containing a GH2 domain, as well as the motif will be denoted as belonging to the MGN clade.

The MGN clade may also contain one or more additional motifs. The motif [I/V]RX[A/C/S]HYP[N/P/D/Q/T/S][D/H/Q/V] (SEQ ID NO: 22) is located at positions 382 to 390 in SEQ ID NO: 3, as well as motifs YGG[D/N]X[G/D][E/D] (SEQ ID NO: 23), corresponding to positions 577 to 583 in SEQ ID NO: 3, and GXXXW[D/E][W/F/Y]X[D/E/N][Q/E/H]] (SEQ ID NO: 24), corresponding to positions 558 to 567 in SEQ ID NO: 3. The motifs are found near the active site of SEQ ID NO: 3 and involved in substrate binding.

### Example 11 (Comparative Example)

Lactases which are not GH2 of Clade DYLGE

Many prior art lactases having, e.g., high thermostability and/or high specific activity are not GH2 lactases of clade DYLGE. A number of these are listed in Table 18.

TABLE 18
Prior art lactases

| Patent Appl. | Seq. | Ref. | Family | Donor | Note |
|---|---|---|---|---|---|
| WO2018189224 | 10 | GENESEQP: BFT09784 | GH42 | Bifidobacterium | increased |
| | | | | catenulatum | thermostability |
| WO2018189224 | 13 | GENESEQP: BFT09787 | GH2 | Lactobacillus | increased |
| | | | clade | delbrueckii | thermostability |
| | | | MGN | subsp. Lactis | |
| WO2018189224 | 16 | GENESEQP: BFT09790 | GH2 | Lactobacillus | increased |
| | | | clade | delbrueckii | thermostability |
| | | | MGN | subsp. Lactis | |
| WO2018189224 | 19 | GENESEQP: BFT09793 | GH2 | Lactobacillus | increased |
| | | | clade | delbrueckii | thermostability |
| | | | MGN | subsp. Lactis | |
| WO2018189224 | 25 | GENESEQP: BFT09799 | GH2 | Lactobacillus | increased |
| | | | clade | delbrueckii | thermostability |
| | | | MGN | subsp. Lactis | |
| WO2018189224 | 26 | GENESEQP: BFT09800 | GH2 | Lactobacillus | increased |
| | | | clade | helveticus | thermostability |
| | | | MGN | | |
| WO2018189224 | 27 | GENESEQP: BFT09801 | GH2 | Lactobacillus | increased |
| | | | clade | helveticus | thermostability |
| | | | MGN | | |
| WO2018189224 | 7 | GENESEQP: BFT09781 | GH2 | Bifidobacterium | increased |
| | | | clade | bifidum | specific |
| | | | MGN | | activity |
| WO2018189224 | 8 | GENESEQP: BFT09782 | GH2 | Bifidobacterium | increased |
| | | | clade | bifidum | specific |
| | | | MGN | | activity |
| WO2018189224 | 12 | GENESEQP: BFT09786 | GH2 | Lactobacillus | increased |
| | | | clade | delbrueckii subsp. | specific |
| | | | MGN | Bulgaricus | activity |
| WO2018189224 | 14 | GENESEQP: BFT09788 | GH2 | Lactobacillus | increased |
| | | | clade | delbrueckii subsp. | specific |
| | | | MGN | Bulgaricus | activity |
| WO2018189224 | 15 | GENESEQP: BFT09789 | GH2 | Lactobacillus | increased |
| | | | clade | delbrueckii subsp. | specific |
| | | | MGN | Bulgaricus | activity |
| WO2018189224 | 17 | GENESEQP: BFT09791 | GH2 | Lactobacillus | increased |
| | | | clade | delbrueckii subsp. | specific |
| | | | MGN | Bulgaricus | activity |
| WO2018189224 | 18 | GENESEQP: BFT09792 | GH2 | Lactobacillus | increased |
| | | | clade | delbrueckii subsp. | specific |
| | | | MGN | Bulgaricus | activity |
| WO2018189224 | 22 | GENESEQP: BFT09796 | GH2 | Bifidobacterium | increased |
| | | | clade | longum | specific |
| | | | MGN | | activity |
| WO2018189242 | 33 | GENESEQP: BFT09807 | GH2 | Bifidobacterium | increased |
| | | | clade | adolescentis | specific |
| | | | MGN | | activity |
| WO2020079116 | 11 | AHGP: EFP1G87VWJ | GH42 | Lactobacillus | Increased |
| | | | | gasseri | activity at |
| | | | | | low pH |
| WO2020079116 | 3 | TREMBL: A0A415FM43 | GH42 | Bifidobacterium | Increased |
| | | | | adolescentis | activity at |
| | | | | | low pH |
| WO2020079116 | 7 | TREMBL: A0A0M4MLP4 | GH42 | Bifidobacterium | Increased |
| | | | | bifidum | activity at |
| | | | | | low pH |
| WO2020079116 | 5/6 | GENESEQP: BFT09779 | GH2 | Lactobacillus | Increased |
| | | | clade | amylovorus | activity at |
| | | | MGN | | low pH |

A number of bacterial GH2 family lactase enzymes of clade MGN were UHT treated in the same way as the GH2 clade DYLGE lactases in Example 4 (Tables 7-8). The data are shown in Table 19.

TABLE 19

| | | | | residual | residual | | | |
|---|---|---|---|---|---|---|---|---|
| | | | | lactose | lactose | gram | % residual | % residual |
| | | | mg | (%) | (%) | lactose/mg | activity | activity |
| | SEQ | Donor/meta- | ep/L | after | after | ep (from | after | after |
| Enz# | ID NO | genome | milk | 0.5 hr | 72 hr | 0.5 to 72 h) | 0.5 h | 72 h |
| 40 | 3 | Lactobacillus | 15 | 4.375 | 4.227 | 0.098 | BD | BD |
| | | delbrueckii subsp. | | | | | | |
| | | bulgaricus | | | | | | |
| 41 | 25 | metagenome A | 12.7 | 4.644 | 4.607 | 0.029 | BD | BD |
| 42 | 26 | metagenome A | 12.7 | 4.664 | 4.649 | 0.012 | BD | 0.4 |
| 43 | 27 | metagenome B | 12.7 | 4.677 | 4.677 | 0.0004 | BD | BD |
| 44 | 28 | metagenome B | 12.7 | 4.669 | 4.668 | 0.0011 | BD | BD |
| 45 | 29 | metagenome C | 12.7 | 4.676 | 4.673 | 0.0024 | BD | BD |
| 46 | 30 | metagenome C | 12.7 | 4.676 | 4.675 | 0.0004 | BD | BD |

BD = below detection level

Almost no change in lactose level was seen after 72 h and therefore also very low gram lactose converted per mg enzyme protein after UHT treatment (calculated as (“g/L lactose at 0.5 h” minus “g/L lactose at 72 h”)/“mg ep/L”, f.ex. for Enzyme #40 (43.75−42.27)/15=0.098 g lactose/mg ep). Likewise, very low residual activity was seen. For all but one (Enzyme #42), the residual activity was below detection level.

These data show that all of the GH2 clade MGN lactase enzymes tested have a poor survival after UHT treatment compared to the GH2 clade DYLGE lactase enzymes tested in Example 4, even though GH2 clade DYLGE and GH2 clade MGN both belong to the GH2 family.

1. A method of producing a lactose-reduced heat-treated milk-based product which comprises:
a) adding an enzyme having lactase activity to a milk-based substrate comprising at least 2% lactose (w/w),
b) after addition of the enzyme, performing a heat treatment of the milk-based substrate by holding said milk-based substrate at a holding temperature of at least 120° C. for a holding time of at least 1 second followed by cooling to produce a heat-treated milk-based product, and
c) storing the heat-treated milk-based product for at least 4 days at a temperature of at most 40° C.,
wherein after step b) but before step c) the lactose content in the milk-based product is at least 0.5% (w/w), preferably at least 1% (w/w), and wherein after step c) the lactose content in the milk-based product is at most 0.2% (w/w).

2. The method of claim 1, wherein step b) is performed immediately after step a) without a dedicated incubation step after step a) and before step b).

3. The method of claim 1, wherein pumping equipment has been fitted to add the enzyme to the milk-based substrate while said milk-based substrate is streaming through process equipment, such as process pipes.

4. The method of claim 1, wherein the time from addition of the enzyme until the holding temperature of step b) is reached is at most 5 minutes, at most 2 minutes, at most 1 minute.

5. The method of claim 1, wherein the time from addition of the enzyme until the heat-treated milk-based product has been cooled to a temperature of at most 40° C., at most 35° C., most 30° C., is at most 3.5 minutes at most 3 minutes, at most 2.5 minutes, at most 2 minutes or at most 1 minute.

6. The method of claim 1, wherein the milk-based substrate comprises 2-30%, 2-17%, more preferably 4-5.5% lactose (w/w).

7. The method of claim 1, wherein the milk-based substrate is milk comprising 4-5.5% lactose (w/w).

8. The method of claim 1, wherein the heat treatment is an ESL treatment, an ultra-pasteurization or a UHT treatment.

9. The method of claim 1, wherein the heat treatment is a UHT treatment performed at a temperature of 128-132° C. for 25-35 seconds, at a temperature of 138-140° C. for 2-5 seconds or at a temperature of 144-146° C. for 1-2 seconds.

10. The method of claim 1, where after the holding time of step b), the milk-based substrate is cooled to at most 40° C. at most 35° C., at most 30° C., within 5 minutes, within 3 minutes, within 2 minutes, within 1 minute.

11. The method of claim 1, wherein after step b) but before step c) the milk-based product is aseptically packed.

12. The method of claim 1, wherein after step b), the enzyme retains at least 0.1% of its initial activity, at least 0.5%, at least 1%, at least 2%, at least 10%, at least 50%, at least 80%, at least 90%.

13. The method of claim 1, wherein the enzyme having lactase activity comprises in its amino acid sequence the motif WTXXDY[I/L/R]GE[P/S/A] and also at least one of the motifs SR[W/Y/F]YSGSGX[Y/G]R and/or [L/V/I]X[L/V/I]PHD.

14. The method of claim 1, wherein the enzyme having lactase activity has a temperature optimum of 30-60° C., 35-55° C.

15. The method of claim 1, wherein the enzyme having lactase activity has a residual activity of at least 0.5% at least 1%, at least 2%, at least 3%, at least 5%, at least 10%, after incubation in skimmed milk having a lactose content of 4.7% at 90° C. for 30 seconds, at 140° C. for 5 seconds and at 70° C. for 30 seconds followed by cooling to 0-10° C. and subsequent incubation at 23° C. for 72 hours, wherein the residual activity is relative to the activity of the same enzyme in skimmed milk without incubation at 90° C. for 30 seconds, at 140° C. for 5 seconds and at 70° C. for 30 seconds followed by cooling to 0-10° C. and subsequent incubation at 23° C. for 72 hours.


SEQUENCE LISTING (30 sequences)

SEQ ID NO: 1 - protein, 1304 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGNVTMNLTTKVANDTKAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGADKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVDWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSERVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERD

SEQ ID NO: 2 - protein, 1710 aa, Bacillus circulans
MSKTTSAAGNSVSYDGERRVNFNENWRFQRETNGSIAGAQNPGFDDSSWRKLNLPHDWSIELDFNKNSLATHEGGYLDGGIGWYRKTFTIPESMKGKRISLDFDGVYMNSTTYLNGEVLGTYPFGYNAFSYDISDKLYKDGRANVLVVKVNNTQPSSRWYSGSGIYRNVYLTVTDPIHVARYGTFVTTPNLEKSIKEDRADVNIKTKISNDAAEAKQVKIKSTIYDGAGNTVQTVETEEKTAAAGTVTPFEQNTVIKQPKLWSIDKPYRYNLVTEVIVGGQTVDTYETKFGVRYFKFDENEGFSLNGEYMKLHGVSMHHDLGALGAATNARGVERQMQIMKDMGVNAIRVTHNPASPELLEAANKLGLFIIEEAFDSWAQSKKPYDYGRFFNAWAEHDIKEMVDRGKNEPAIIMWSIGNEIYDTTNAAGVETARNLVGWVKEIDTTRPTTIGEDKTRGDKVNVTPINSYIKEIFNIVDVVGLNYSENNYDGYHKQNPSWKLYGSETSSATRSRGVYTHPYQYNQSTKYADLQQSSYDNDYVGWGRTAEDAWKYDRDLKHIAGQFIWTGFDYIGEPTPYYNSYPAKSSYFGAVDTAGFPKDIFYYYQSQWKKEPMVHLLPHWNWKEGEKVRVLAYTNASKVELVLNGESLGEKNYDNKQTSWGAPYKETKDGKTYLEWAVPFKPGKLEAVAKDENGKVIARDQVVTAGEPASVRLTADRKVVKADGTDLSFITADIVDSKGIVVPDADHLITFNVTGQGELAGVDNGNASSVERYKDNKRKAFSGKALAIVQSSKLSGKITVHASVAGLSSDSTSVFTVTPADHDKKIVAGIDDVNLTVDVNEAPKLPSEIKVYYSDESAAAKNVTWDEVDPKQYSTVGEFTVEGSVEGTSLKAKAFVIVKGIVAVKPYSTATKVGVQPVLPEKATLLYSDGTTKGATVTWDEIPEDKLAKEGRFTVEGSVEGTDLKANVYVRVTNEVKSVNIMLQEQGSAYPKLEATFTNPADNLQHLNDGIKSYTNNPVNRWTNWTRTPRDAGDSITVNFGKKHVINNLDLFVFTDSGTVVPEKAEVQYWDGTAWKDVENLTQPSPYVVEKNELTFDAVATEKLKFHLTPSVKGKFLALTEAEVYADQIVMGETAKLQSITVNGKALEGFDHAKKNYELVLPYGSELPKIEAAAADNATVTILPAFSYPGTAKLFVTSEDGKVTTEYSIGVSTEEPKLVSAELSADKTNVMEDDIIDLKVIGLFESKEKIDVTDSQPTYEFDQQIIKIEGNKLYALETGNVKVKVTVTYKGVSVTTPALEFTIAKNPAPKYITSLEPVTVVVKKGEAPELPATVVAHYNRGIPRDVKVKWERINPSKYQQLGEFTVSGMVEGTDIKAQAKVIVKGAVAVEDIRMAVLLKQMPQLPGKVTVYYSDGAEEQRAVKWEEIPQEELENVGEFKVKGDVNGVKLKATATIRVTDEVGGEQNISRAKNGYEYPKAEASFTNNGPGSSDRIEAINDDVISYEANPHNRWTNWQPVPRAGDWVSITFGDYEPTEYDVDSMEIHWFADHGTSYPERFQIEYKSGDSWKEVTSLKSDPASPALGKANVYSFDRVKTSAIRVKMTAQAGKSLAITELKVFSKWPKAGTEPEVTDIKVGGKSILEDFEQKGDHYEVTIDAGDANVMPKINVKAKDQTSITIVPAVTSPSTAKVIAKSEDGKKVKVYSIHYK

SEQ ID NO: 3 - protein, 1008 aa, Lactobacillus delbrueckii
MSNKLVKEKRVDQADLAWLTDPEVYEVNTIPPHSDHESFQSQEELEEGKSSLVQSLDGDWLIDYAENGQGPVNFYAEDFDDSNFKSVKVPGNLELQGFGQPQYVNVQYPWDGSEEIFPPQIPSKNPLASYVRYFDLDEAFWDKEVSLKFDGAATAIYVWLNGHFVGYGEDSFTPSEFMVTKFLKKENNRLAVALYKYSSASWLEDQDFWRMSGLFRSVTLQAKPRLHLEDLKLTASLTDNYQKGKLEVEANIAYRLPNASFKLEVRDSEGDLVAEKLGPIRSEQLEFTLADLPVAAWSAEKPNLYQVRLYLYQAGSLLEVSRQEVGFRNFELKDGIMYLNGQRIVFKGANRHEFDSKLGRAITEEDMIWDIKTMKRSNINAVRCSHYPNQSLFYRLCDKYGLYVIDEANLESHGTWEKVGGHEDPSFNVPGDDQHWLGASLSRVKNMMARDKNHASILIWSLGNESYAGTVFAQMADYVRKADPTRVQHYEGVTHNRKFDDATQIESRMYVPAKVIEEYLTNKPAKPFISVEYAHAMGNSVGDLAAYTALEKYPHYQGGFIWDWIDQGLEKDGHLLYGGDFDDRPTDYEFCGNGLVFADRTESPKLANVKALYANLKLEVKDGQLFLKNDNLFTNSSSYYFLTSLLVDGKLTYQSRPLTFGLEPGESGTFALPWPEVADEKGEVVYRVTAHLKEDLPWADEGFTVAEAEEVAQKLPEFKPEGRPDLVDSDYNLGLKGNNFQILFSKVKGWPVSLKYAGREYLKRLPEFTFWRALTDNDRGAGYGYDLARWENAGKYARLKDISCEVKEDSVLVKTAFTLPVALKGDLTVTYEVDGRGKIAVTADFPGAEEAGLLPAFGLNLALPKELTDYRYYGLGPNESYPDRLEGNYLGIYQGAVKKNFSPYLRPQETGNRSKVRWYQLFDEKGGLEFTANGADLNLSALPYSAAQIEAADHAFELTNNYTWVRALSAQMGVGGDDSWGQKVHPEFCLDAQKARQLRLVIQPLLLK

SEQ ID NO: 4 - protein, 1298 aa, Bifidobacterium samirii
DDILSRADSTTQMSSDPEVVYANAVTGTAERSMNFDANWRFLLSDSVSAQDPGFDDSQWRQISLPHDYSIEQDYSTSGEAESAYLPGGTGWYRKQFTLSRDLAGKRVSIGFDGVYMNATVWINGHKLGTHPYGYSPFSFDLTDHAVFGGENTIVVKVENKTPSSRWYSGSGIYRSVTLNVTDAVHVDTGGVRVTTPDLAAQHGGDVTVNLGADIANDSGAPVEVTLRQSVFAKGEGTERAIGTSVGEPTTIAAGASAHIDSTLTASSPKLWSTQNPNLYTVRTEVMSGDTVLDTYDTDYGFRWTSFDADGGFSLNGTPTKIKGVSMHHDQGALGAVANRRAIERQVEILKGMGVNSIRTTHNTAARTLIDVCNEQGVLVVEEAFDTWIKAKNGNSEDYGKWFSVEVGDGNAIEGGDASGTWALFDLTAMIRRDANDPSVIMWSLGNEMMEGISGSVSEYPDVARRLIAWTNEADPTRPATLGDNKIKAGWSESTAMADALTDSDGVVGTNYSSGGNYDSVHAAHPDWRLYGSETASAINSRGIYDRTTGGSQTADKQLTSYDASAVGWGATASSAWYDVITRDFVAGEYVWTGFDYLGEPTPWNGTGSGAQGAWPSPKSSYFGIVDTAGLPKDSYYFYQSQWNDDVHTLHVLPAWNENVVAKDSQGKVPVVVYSDAAAVELFFTPKGGERTSLGRRQFTTKTTAAGYRYQVYEGEGKSSSADKNLYLTWNVPWAEGTITAVAYDESGAVIADTEGRSSVTTTGEASALTAEADRTTIEADGTDLSYVTVDVTDADGNTVPDAADRVTFDVKGAGRLVGVDNGSSPDHDSYQANNRRAFSGRLVAIIQSTDKAGDITVTATADGLKSSTTTITTTAVPGASTERAVDGLYYARNYYVKTGNSPRLPDTIEVRYTDGTSERQAVTWNEVGDDQTAVPGTFTVTGTVGEQTATVRVTMIDDGGALLNYSATTPVGIAPALPESRPLVLADGTVTNAGFAVSWTTPDDSAYAEAGTVTVPGTATVFDRTVDVSATIRVQQESVAIGDSVSGSALNLTQSVPADLQTDTLDAIKDGSATADPNTGGGANPSVWTNYNWAQSGRDGSDKSELTFEYATQQKFGRFVMHFYTDSYSARLPKNVDIQVSDDGANWTTLSTTATKGETRDNVTPYTYDFAPVTATFVRFAITNSDEALSGRRPCTGISEIELKLAVTDFTTNTDAGLSSLTVNGTDVPASALAAGSWNTPAIRADVEAVGAGNASVTVLPAHEGVIRVITESEDHATRGVFAVNLGREQSDAADSDA

SEQ ID NO: 5 - protein, 1307 aa, Urmitella timonensis
AGNTDYSHLNTSAQMVSSAQSVPLTTIDGKARFQSLNNGWKFNLGDISGAQATAYNDAKWRTVDIPHDYSIEQPFSKSMEGESGYLPGGVGWYRKSLNLPSELKNKRIKLSFDGIYMDATIYVNGTELANHPYGYTPFSIDITDNVKFGGNNVIAVKVNHQTPSSRWYSGSGIYRDVDLVITDPIHIASSGVKITPKNIDTYTEAGVLNLDVSTKLINAATSPKTITLRHTISPKNGGNSIGTVTSTPITVEAGQSLTDSTALSAQSPQLWTIEKPVQYLVKTEVFEGSKLLDTLETNTGFRQLTFDPNTGFALNGKKMKLKGVSMHHDQGALGAKAYRDAIARQIDILKKMGANTIRITHNPASRDLIDLANQKGMLLIEEIFDGLHCAKNGNQMDYARFFNQLVPKDSKLENVTAGSTWARFDLETTMRRDINAPSIIMWSLGNEIQEGTTCGIDSFLSPQTNLIKWAAEIDNTRPLTRGDNAIKHNNSAINVMKSLVSTAKTSGTTGAVGANYANGGQYDSIHRQIPGAMLYGAETASSVNSRGVYSRTTDNRQTADKQLTSYDNSKVNWGATAAQAWLDVIKRDFVAGTAVWTGFDYLGEPTFWNGTSPGPQGQWPSPKNSYFGIIDTAGFPKDSYYLYQSQWNDKVTTLHVLPAWNRSVLQNQGNGNQKVVVYSDASAVELFFTGTDGKKKSLGKKTLAKKTTPAGYKYQLYEGSDKSSIESENLYLSWNVQYADGTLSAVAYDESGNVITNTQGRDTVSTAGKAAKLDVSLNRTELSANNQDLAYVEVTVTDDNGTPVPDAALKVDFNASGACEMVGTDAGKQDDLTSYLSSSRNTYNGKVLGIVKSKKIAGQCTITVSGTNLPKVTKTVTVTPAPNASQDRAIDAYSYPRNYYVQVGTQPRLPDKIDAIFTDGSQDAVEVTWNQVPKEQYSKAGFFTVLGKTKHGHQVSANVTMLNRVGAVLNYSATTPVGIVPTIPATRPAVLPDGTILDVQFDVAWQKPNEKVYSKAGTVTIEGKAKFLDKELPVTATIRVQEAVVKIGDSVSGAATLSQDIPADKQSDTLDAIKDGNTAVGSNTQGGANPTAWTNYTNSQAGDNSAAIEFKYATQQTFGEFNVYFFRDSYSARYPDGGTTKFYVKEAAGDQWKPVETTESIATSEHPENVKKYSYALKKPVNATYVKLEVTNKSEQLSGRNPCTGITEVELKTASNTTSTFSTAKLASLQVNGENLSKSVLKSFEYATPAEKITQIKALAKDNAAITVLPEYEGVARVIIESEDHKTINTFKIYLGKEQPEPADDAS

SEQ ID NO: 6 - protein, 1309 aa, Varibaculum sp.
AGNTDYSHLNTSAQMVSSAQSVPLTTIDGKSRFQSLNNGWKFNLGDISGAQATAYNDAKWRTVDIPHDYSIEQPFSKSMEGESGYLPGGVGWYRKSLNLPSELQHKRIKLSFDGIYMDATIYVNGTELANHPYGYTPFSIDITDNVKFGENNVIAVKVNHQTPSSRWYSGSGIYRDVDLVITDPIHIASSGVKITPKNIDTYTEAGSLDLDLSTKIINAAASPKTITLRHTISPKTGGNSIGTVTSTPITVEAGKSLTNSTALSAQSPQLWTIEKPVQYLVKTEVFEGSRLLDTLETNTGFRQLTFDPNTGFALNGKKMKLKGVSMHHDQGALGAKAYRDAIARQIDILKKMGANTIRITHNPASRDLIDLANQKGMLLIEEIFDGLHCTKNGNQMDYARFFNQLVPKDSKLENVTAGSTWARFDLETTMRRDINAPSIIMWSLGNEIQEGTTCGIDSFLSPQTNLIKWAAEIDNTRPLTRGDNAIKHNNSAINVMKSLVSTAKTSGTTGAVGANYANGGQYDSIHRQIPGAMLYGAETASSVNSRGVYSRTTDNRQTADKQLTSYDNSKVNWGATAAQAWLDVIKRDFVAGTAVWTGFDYLGEPTFWNGTSPGPQGQWPSPKNSYFGIIDTAGFPKDSYYLYQSQWNDKVTTLHVLPAWNRSILQNQGNGNQKVVVYSDASAVELFFTGTDGKKKSLGKKTFAKKTTPAGYKYQLYEGSDKSSIESENLYLSWNVQYADGTLSAVAYDESGNVITNTQGRDTVSTAGKAAKLDVSLNRTELSANNQDLAYVEVTVIDDNGTPVPDANLKVDFNASGACEMMGTDAGKQDDHTSYLSSSRNTYNGKVLGIVKSKKIAGQCTINVSGTNLPKVTKTITVTPAPNASQDRAIDAYSYPRNYYVQVGTQPRFPDKIDAIFTDGSQDAVEVTWNQVPKEQYSKAGFFTVLGKTKHGHQVSANVTMLNRVGAVLNYSATTPVGIVPTIPATRPAVLPDGTILDVQFDVTWQKPNEKVYSKAGTVTIEGKAKFLDKELPVTATIRVQEAVVKIGDSVSGAATLSQDIPADKQSDTLDAIKDGNTAVGSNTQGGANPTAWTNYTNSQAGDNSAAIEFKYATQQTFGEFNVYFFKDSYSARYPDGGTTKFYVKEAAGDQWKPVETTESIATSEHPENVKKYSYALKKPVNATYVKLEVINKSEQLSGHNPCTGITEVELKTASNTTSTFSTAKLASLQVNGENLSKSVLKSFEYATPAEKITQIKALAKDNAAITVLPEYEGVARVIIESEDHKTINIFKIYLGKEQPEPADDASRD

SEQ ID NO: 7 - protein, 1291 aa, Clostridium nexile
SGQVQVRTDSNTQMSSDKEVVYVNTYDTSKRVHDFDANWKFYLGDADGAEAVNFDDSAWRTVSVPHDYSIEQEYTQSGEAESGYLPGGTGWYRKHFTLTEDMKDKEIRLDFGGVYMNATVWVNGTQLGTHPYGYTPFSFDITDYVKAGEENVITVKVDHQTPSSRWYSGSGIYRSVDLTVMDKVHVDLNGTKIATPNLAKEKDGDVNMDIVTTVANEEKEAKEIILVHTLREKGKKDAVATVTTERQSVEAGKTAGIKANLKVNQPKLWGLGEKNAHLYTVTTEVKVNDKVVDTYDTDYGFRYTELDTKTGFSLNGEKVKLQGVCMHHDQGALGAEAQKRAIERQVEILQEMGCNSIRVTHNPAAEELIEVCNEKGILVINELFDGWHKAKNGNVNDYAKWFNQTIGDENKILDGKSDMTWAEFDLKSTLNQGKNDPSIIMWSLGNEISEGASEGGYYEISKNLIKWAQEIDTSKPLTIGSNRVKGAIGQADNEHIKIANDLTKIGGMSGTNYSGGSNYDALHSTYPEWFLYGSETASAVNSRGVYTTKANAALDQDKQLTSYDKSKVNWGALASEAWYDVITRDFVAGEYVWTGFDYIGEPTPANKINAGAATSWPSPKNSYFGIVDTAGLPKDSYYFYQSQWNDEVNTLHVLPTWNEDSLMKDENGNVEVVVYSDAAKVKLFLNDKEIAEQTFEEHTTGAGYKYQTVKGKTGHESMYMTFSVPYEKGTLKAVAYDKDGNEIKNTQGRSSVTTTDKATKLDATVDRNSIKADGKDLSYITVDVTDANGNIVPDADNNVRFTVEGAGTLVGVDNGKQADHQSYQDDNRDAYNGSLVAIVQSTKDAGKITVKAEADGLTLDTVEITTTEVEDGTVDKTQVDSFYMSKHYYVKAGSKVQLPETIKTRFKDGTEKDLPVKWDEIKETTGSFVVNGLVDGKYKVSVTVNVIDQLGGLMNYSATTPKGTEPNLPTTRPAIAQDGTVLNVSFPVTWGKVDKTKYDEPGIVTVNGTSNVLGTAMEVTASIRVQEETYEIVGSVDNPSQLTQDIEKDKQSDTLDAVWDGKTAIADNTSGGPNPTAWSNYKNSQAGDNTAEITFQYATQQVIGEVTVHFFKDSASARYPKAGTTKLYVKANDGDDWQEVVAEETIGEENGRVKAYTYKFNPVGATLFKVAVTNSDEVLTTGHKPCTGITEIEIKKAVGSYKTNTTAKLASLEVNGKALTAEELAENAYYTLDKTATVTAAGADNAAVTVLPVHNKQIRILIESEDHRTKNTFVIYLEQEKPMSPDDDSKD

SEQ ID NO: 8 - protein, 1313 aa, Bacillus sp.
AEKTNASTENSVTYEGERRINFNGNWRFQRETTGSIAGAQNPGFDDSSWRQLNLPHDWSIELDFNKNSLATHEGGYLDGGIGWYRKTFTVPESMKGKRISIDFDGVYMNSTTYLNGQLLGTYPFGYNAFSYDITDKLYRDGRENVLVVKVNNTQPSSRWYSGSGIYRNVYLTVTNPVHVARYGTFVTTPNLETAYAAGKADVNIKTKVSNDSGEAVQVKVKSTIYDDAGKAVATVESEEKTAAAGAETQFENNTIIEQPKLWDIDNPYRYKLVTEVIARGQSVDTYETRFGARYFNFDENEGFSLNGKYMKLHGVSMHHDLGALGAATNARAVERQMQIMKDMGVNAIRVTHNPASPELLEAANKLGLFIVEEAFDSWAQSKKTYDYGRFFTTWAEHDIKEMVDRGKNEPAIIMWSIGNEIYDTTNAAGVETARNLVRWVKEIDTTRPTTIGEDKTRGDKVNVTPINSYIKEIFNIVDIVGLNYSENNYAGYHQQNPNWKLYGSETSSATRSRGVYTHPYQYNQSTKYADLQQSSYDNDYVGWGRTAEDAWKYDRDLKHIAGQFIWTGFDYIGEPTPYYNAYPAKSSYFGAVDTAGFPKDIFYYYQSQWKKEPMVHLLPHWNWTEGETVRVLAYTNAYKVELFLNGESLGVRSYENKKTSWGAPYKETKDGKTYLEWAVPFKAGTLEAVAKDESGKEIARDQVVTAGEPAAVKLTADHKVITADGTDLSFITAEIVDSKGIVVPNADHLIKFNVSGQGELAGVDNGNASSVERYKDNKRKAFSGKALAIIQSNKLAGEITVNASVAGLSGDTTSLFTVAPADRDKKTVVGMDTVHVTIDVDQAPKLPTELKVYYSDATVDAKKVTWDLVDPKQYSSVGIFTVEGKVEGTSLKAKAVVTVKGIVAVKPVSTATKVGVRPVLPENVALLYSDETTKDVNVSWEEISADQLAKEGRFVVEGSVEGTDLKAKAFVRVTNESKPVNIMLPAQGSTYPQLEATFTNPADNLQHLNDGIKSYTNSPVNRWTNWTRTPRDAGDSITVNFGKQHVINNLDLFVFTDSGTVVPDTAAVQYWDGTAWKDVENLTQPSPYVVEKNEIRFDAVTTDKLKFHLIPSVKGKYLALTEAEIFADQMVMSSTASLSSIIVNGKPLAGFDAAKKTYDLVLPYGSDLPKIEAEAADNAAVTILPAFTYPGTAKLFVTSEDGKVTSEYSIQVSMEEPKLVAAKLSVDQTKVVEDDIIDLKVEGTLESGEVIDLTDSEPTYEFDNQNIKIEGNKLYALETGEVKVKAVVKYKDVTVTTPEMTISIAKNPAPKYI

SEQ ID NO: 9 - protein, 1130 aa, Neobacillus bataviensis
AEKTNATTENSLHFEGERRMNFNGNWRFQRETNGSIAGVQNPGFDDSSWRQLNLPHDWSIELDFNKNSLATHEGGYLDGGIGWYRKTFSVPAAMKGKRISIDFDGVYMNSTTYLNGQLLGTYPFGYNAFSYDITDKLYTDGRENVLVVKVNNTQPSSRWYSGSGIYRNVYLTVTNPVHVARYGTFVTTPNLETASAAGKADVNIKTKISNDSGEAAQVKVKSTIYDEAGKAVSTVESEGKTAAAGAEAHFENNTIIEQPKLWSIDNPYRYKLVTEVIVGGQTVDTYETRFGARYFKFDENEGFSLNGKYMKLHGVSMHHDLGALGAATNARAVERQMQIMKDMGVNAIRVTHNPASPELLEAANKLGLFIVEEAFDSWAQSKKTYDYGRFFTTWAEHDIKEMVDRGKNEPAIIMWSIGNEIYDTTNAAGVETARNLVRWVKEIDTTRPTTIGEDKTRGDKVNVTPINSYIKEIFNIVDIVGLNYSENNYAGYHQQNPNWKLYGSETSSATRSRGVYTHPYQYNQSTKYTDLQQSSYDNDYVGWGRTAEDAWKYDRDLKHIAGQFIWTGFDYIGEPTPYYNAYPAKSSYFGAVDTAGFPKDIFYYYQSQWKSEPMVHLLPHWNWTEGESVRVLAYTNAYKVELFLNGESLGERSYENKKTSWGAPYKETKDGKTYLEWAVPFKAGTLEAVAKDESGKEIARDQVVTAGSPAAVKLTADRKVISADGTDLSFITAEIVDSKGIVVPNADHLIKFNVSGQGELAGVDNGNASSVERYKDNKRKAFSGKAVAIIQSNKLAGEITVNASVAGLSGDTASLYTVAPADRDTKTVVGIDAVHVTVDVNEAPKLLSEIKVYYSDATVASKRVTWDAIDPKRYSTVGTFTIEGSVEGTSLKAKAFVTVKGIVAVKPYSTATKVGVQPVLVEKVTLLYSDETTKDVNVTWEDIAADQLAKEGRFVVEGSVEGTDLKAKAFVRVTNESKQVNIMLPAQGSTYPKLEATFTNPDDNLQHLNDGIKSYNNNPVNRWTNWTRTPRDAGDSITVNFDKQHVINNLDLFVFTDSGTVVPNKAAVQYWDGTAWKDVENLTQPSPYSVEKNEIRFDAVSTDKLKFHLTPSVKGKFLALTEAEIFADQI

SEQ ID NO: 10 - protein, 1313 aa, Neobacillus mesonae
MSKTTSAAGNSVNYDGERRMNFNENWRFQRETNGSIAGAQNPGFDDSSWRQLNLPHDWSIELDFNKNSLATHEGGYLDGGIGWYRKTFTIPESMKGKRISLDFDGVYMNSTTYLNGEMLGTYPFGYNAFSYDITDKLYKDGRPNVLVVKVNNTQPSSRWYSGSGIYRNVYLTVTDPIHVARYGTFVTTPNLEKTIKEDRADVNIKTKISNDSVETKQVKIKSTIYDEAGNAVQTVESEEKAAAAGTVTPFEQNTVIKQPKLWSISKPYRYRLVTEVIVGGQTVDTYETKFGARYFKFDENEGFSLNGEYMKLHGVSMHHDLGALGAATNARGVERQMQIMKDMGVNAIRVTHNPASPELLEAANKLGLFIVEEAFDSWAQSKKTYDYGRFFNSWAEHDIKEMVDRGKNEPAIIMWSIGNEIYDTTNATGVETARNLVRWVKEIDTTRPTTIGEDKTRGDKVNVTPINRYIKEIFDIVDVVGLNYSENNYDGYHKQNPNWKLYGSETSSATRSRGVYTHPYQNNQSTKYPDLQQSSYDNDYVGWGRTAEDAWKYDRDLKHIAGQFIWTGFDYIGEPTPYYNSYPAKSSYFGAVDTAGFPKDIFYYYQSQWKKEPMVHLLPHWNWKEGEKVRVLAYTNASKVELFLNGESLGEKGYENKQTSWGASYKETKDGKTYLEWAVPFKPGKLEAVAKDENGKVIARDQVVTAGEPAAVRLMADRKVVKADGTDLSFITAEIVDSKGIVVPNADHLIKFNVTGQGELAGVDNGNASSVERYKDNKRKAFSGKALAIVQSSKLSGKMTVHASVAGLSSDSTSVFTVTPADHDQKTAAGMDDVRLTVDVNEAPKLPSEIKVYYSDESAAVKKVTWDAVDPKQYSTVGEFTVEGSVEGTSLKAKAFVIVKGIVAVKPYSTATKVGVQPVLPEKATLLYSDETTKAAAVTWDEIASDKLAKEGQIVVEGSVEGTDLKAKAYVRVTNEVKSVNIMLQEQGSAYPKLEATFTNPDDNLQHLNDGIKSYNNNPVNRWTNWTRAPRDAGDSITVDFGKKHVIDNLDLFVFTDSGTVVPEKAAVQYWDGTAWKDVENLTQPSPYVVEKNKLRFDAVETDKMKFHLTPSVKGKFLALTEAEVYADQIAMGASAKLENITVNGKALEGFDPAKKNYELVLPYGSELPKIEAAAADNATVTILPAFSYPGTVKLFVTSEDGKITSEYSIRVSTEEPKLVSAELTVDKTNLIEDDVIDLKVIGLFESKEKIDVTDSQPTYEFDQQIVKIEGNKLYALEAGKVEVKATVTYKGVSVTTPAVTFTIAKNPAPKYI

SEQ ID NO: 11 - protein, 1288 aa, Streptococcus entericus
TESPEEWHKVNHYNDTDKVVNFDKDWKFNLGDSAAASSKSFDDSSWKKLDLPHDFSLIQDYTPSGEAESGYKPGGIGWYRKSFTIGEEAAKGRVNLQFDGAYMETEVFINGTSLGTHTYGYSPFSFDLTEHLKKDEENVLAVKVTNPIPSSRWYSGSGIYRSVHLEFAPSVHLAEYGVVVTTPDLKDTHNLDSGSRINISTTVVNQGEDAAQVTVRSTLFERDSNGGRGRQVAVSTDVPAKSVTAGSSSAIDTSLTIVKPKLWSVDNPHLYVLRTEVLKDGQVIQSREQETGFRYTAFDKDTGFKLNGQNLKLQGVSMHHDQGGLGARAYYDAIERQFTILKDMGVNAVRVTHNPASRAMKDIANRKGMLLIDEAFDTWEHAKNGNTNDYARYFNRVIGDSVRDLNGARADQTWAEYHIKQMVKSGINDPSIIMWSTGNEVMEGFSANTSNYPQVIARLSDWVAEIDPTRPATFGDNMLKNRHQNAIGMANVLTEKKGIVGYNYANGAQYDQGHRDHPDWIIYGSETASAVNSRGVYNVKGNTQRQDRQLTSYDQSKVNWGHVASEAWYDVLTRDFVAGEFVWTGFDYLGEPTPWNKTNPGAQGTWGSPKSSYFGIVDTAGLPKDSYYFYRSQWQKGDTTLHVLPAWEESVVAKDNQNRVEVVVYSNAAKVKLVHISPEGVERVIGTKAFSKETTSAGHTYQVYKGSDKQQVEHKNLYLTWHVPYEAGTIKAIALDEQDRVIEQTVGTSQVSTFGAAHKLQATVTHKKDTVDDHNLAYVTIDVQDAKGNLVVNASNRIKVTVTGEAELVAMDNGNPVDHQSYQDDNRQAFGGKVVAILKMTGKAGQVQVTATADGLASSTQTFEVAAKRQSQADKVDSYKLTKTIYIKKGAELKLPEKTTVRYEDGREVEKDLHFDRSAIAPKLAAGETFTATGRINDLGTRAELVVAVIDQVAAIKNISTATEVGARPKLPETAQAYLADGSLLSATFPVSWQMPEEAQFAQTGTVMINGTADVLGQKLPITASVRVGEKTLTIDQNVAPSARTLTQNIVGSDTLTAINDGNLNVSANNQGGPNPTIWSNWQAAQNGQKTATLSFTYDTAQTIAEVVAYYHRDRLSLRTPKSATFAWNRGSGAEDETIAIASQTAEAVAGTSLTKVTYRLAQPVSAVEFKMTVENSDEAMPSAQKSSVGIAEVQLMTALESFNQHSEAGLTDIRVGDRRYTGNQISTSMRVPATGAVTATNNNRNVGITILPEQDGVVKIFTESEDKTRSAVYSIQLVPENPRTPDD

SEQ ID NO: 12 - protein, 1678 aa, Streptomyces cirratus
DETPVAVATEKVEKETPKEEAVDSKASEQPVTTETVNSEAVETNHEVEKKVEAPTTETTPEKKETSDVEKQAPAVTQDQSAQTEKPELKPATNDEVKQLIEDRKEEFNQNWHFKLNANAKDAVKPDADVSSWKKLDLPHDWSIYNDFDHDSPAQNEGGQLNGGDAWYRKTFKLDEEDLNKNVRITFDGVYMDSQVYVNGQLVGHYPNGYNQFSYDITDYLHKDGRENVVAVHAINKQPSSRWYSGSGIYRDVTLQVTDKVHTEKNGTTILTPDLEKQQNGKVDTHVTSKIVNTDDKDHEILAEYQIIERNGQAVTDLVRTASQVLKAHQSASLNAILQVDKPKLWTVNSDKPALYELVTRVYRDGQLVDAKKDLFGYRYYNWTPNEGFSLNGERIKFHGVSLHHDHGALGAEENYKAEYRRLKQMKDMGVNSIRTTHNPASEQTLQIAAELGLLVQEEAFDTWYGGKKQYDYGRFFDKDATHPDAKKGEKWSDFDLRTMVERGKNNPAIVMWSIGNEIGEADGKPRSLATVKRLVQVIKAVDKTRYVTMGADKFRFGDGSGDHEKIANELDAVGFNYSEDNYKKLRAKHPNWLIYGSETSSATRTRGSYFRPERELVHSNQAYRHYEQSDYGNDRVGWGKTATASWTFDRDNAGYAGQFIWTGTDYIGEPTPWHNQNQTPVKSSYFGIVDTAGIPKNDYYLYQSQWVSAQKKPMVHLLPHWNWENPTLKANVADADGNIPVRAYSNAVSVELFLNGNTLGKKTFTKKQTSDGRTYQEGANANELYLEWKVAFEPGTLEAVARDEAGNIVARDKIVTAGEPAGVRLVKEDHAIAADGKDLTYIYYEIVDSKGNVVPTANNLVRFQLHGQGQIVGVDNGEQASRERYKEQADGSWIRKAFNGKGVVIVKSTEQAGKFTLTAHSDFLKSGQTTIFTGKKDQEEKTVLGTEVPKVRTVIGQAAKMPLKVGFIYSDGSRAKLPVEWSAVDVNQPGIFTVKGVAEGRDVEARVEVLALEAELPVVKRIAPSTDLSTVDPSVSYVLSDGTLGSYDVDRWEVAAEDQAKLTIPGSRIQAKGISGDDEIAATFVVAEGQAASPVVPTVTVADQPVEGLSTDQPVLYHKLAYGTALPEVKALAENAQVTVIQANEANGMRASIYVQPNDGGALQTYAIQFLQEAPQIERLSLEVENADALKEDQTVGIKVLAHYQDGTQAVLKADKVTFSTSGEGDVAVRKGMLELHKPGQVTLKAQFEGAEGQIDLNIQANTENKVVQAIRPVSVVTGLNQEPSLPDTVTVEYDKGFPKVHKVTWQAVAKEDLAKYHSFDVLGKVEGIENEAHAKVSVEGIIAVEEVTTTTPVSEAPQLPESVRTYHSNGQVSSAKVTWDAIDPSQYAQEGSFTVTGHVEGTQLPTKLHVRVSTQTETGANISDQWTGSELPLAFASDSNPSDSVANVNDKLISFNDQPANRWTNWNRTSEASVGILFGDSGILSKRSVDNLNVAFHEDHGVGAPKSYVIEYYVGKDVPTVPKNPSFVASEDHVFNDDNNWKQVTNLKAPNQVRAGEMTHFSFDKVNTYAVRIRMVRPDGKWGTSITEIQIFSKQVAAAKKAQAQIQVDGKDLPNFNPGLTDYYLEAKEGQAPTVTASVSDNGIATVIPSVREGDPVRVVVKAENGDILGEYRLHFT

SEQ ID NO: 13 - protein, 1299 aa, Bifidobacterium aerophilum
DDTATRADSTTQMSSTPEMVYANRVEADQARTTDFDANWKFKMSDAIAAQDPDFDDSAWQRISLPHDYSITQDYTQSGEAESAYLPGGTGWYRKTFTLDRGLAGKQVSVNFDGVYMNATVWLNGVKLGEHPYGYSPFSFDLTKAARFGAENTIVVKVDHKTPSSRYYSGSGIYRDVTLTVTDGVHVAGNGVAVATPELAEQNGGDVTMNLTATVSNDTEAAADVTLKQTVFPKGGETSAAIGTVITEATNVAAGESADVASTITAAQPKLWSVENPNLYTVRTEVQVAGRTVDTYDTTYGFRWTGFDADTGFSLNGVKVKLKGVSMHHDQGSLGAVANHSAIARQVRILKDMGVNSIRTTHNTAAKALIDICNEEGILVIEEIFDGWNRAKNGNTEDYAKYFDKTIASDNRILGGDKDESWAKFDLTATINRDRNDPSIIMWSTGNEMMEGISGGVSDFPDISAKLTRWTNAADATRPATYGDNKIKANWTESNTMGDSLTAAGGVVGANYEDGRLYDSTHQAHPTWKLYGSETASSINSRGIYNVTTGATGDQQLTSYDNYAVGWGATASSAWYDVIRRDFVAGEYVWTGFDYLGEPTPWNGTDSGFKGTWPSPKNSYFGIVDTAGLPKDSYYFYRSQWNTKSHTLHILPAWNGDVVAKDSSGNVPVVVYTDAATVKLYFTPKGSSDKRLIGEKSFTTHTTDAGYTYQTYDGADKNATADKNMYLTWNVKWAEGTITAEAFDKDGRRITDTEGRSRVTTTGVASQLKVEADATKLTADGKSLSYITVDVTDADGNVVPNADDRVTFKVEGAGKLVGVDNGKTTDHQSYQDDNRKAFSGKLVAIVQSTKTAGGITVTATAEGLKPATTTITTTAVPGASTARTIDGFYYARNYYVKTGNKPMLPSDVEVRYSDGTKDRQNVTWNVVTDEASKTGTFTVTGTVADQTISVRVTMIDEGGALLNYSATTQVGMPAVLPESRPLVLSDGTVTSANFAVTWTKPADGEYDEAGTVVVPGIATVFGKEIGVTATIRVQTANVTIGDSVSGNAMNLTQDIPEGSQSDTLDAIKNGSTTVDDNLSGGANPSAWTNWAWSKAGHNTAQITFEYATQQTLGQIVMYFFRDSSSVTFPDAGTTKIEISDNGTDWTRLNVEETIAPAESSNRVKPYTYDFAPTGATFIRFTVTNSETRASSGVTSTGLTEIELKVATTTFTTNTAAGLASLKVNGTAVSESALASGSYNTPAIVADVEAVGKDNASVTVLPAHNDVIRVITESEDHATRETFAINLGKEQEDTADSDARD

SEQ ID NO: 14 - protein, 1309 aa, Neobacillus niacini
TSTTENSVKYEGERRINFNENWRFQRETNGSIAGAQNPGFDDSSWRRLNLPHDWSIELDFNKNSLATHEGGYLDGGIGWYRKTFTVPEAMKGKRISIDFDGVYMNSTTYLNGQLLGTYPFGYNAFSYDLTDKLYTDGRENVLVVKVNNTQPSSRWYSGSGIYRNVYLTVTNPVHVARYGTFVTTPDLETAYAEGEADVNIKTKVSNDSGEAAQVKVKSTIYDESGKAVSTVESEEKTAAAGTAANFDNNTIIEQPKLWSTDNPYRYKLVTEVIVGVQTVDTYETSFGARYFKFDENEGFSLNGTYMKLHGVSMHHDLGALGAATNARAVERQMQIMKDMGVNAIRVTHNPASPELLEAANKLGLFIVEEAFDSWAQSKKTYDYGRFFTTWAEHDIKEMVDRGKNEPAIIMWSIGNEIYDTTNAAGVETARNLVRWVKEIDTTRPTTIGEDKTRGDKVNVTPINSYIKEIFNIVDIVGLNYSENNYAGYHQQNPNWKLYGSETSSATRSRGVYTHPYQYNQSTKYPDLQQSSYDNDYVGWGRTAEDAWKYDRDLKHIAGQFIWTGFDYIGEPTPYYNSYPAKSSYFGAVDTAGFPKDIFYYYQSQWKKEPMVHLLPHWNWTEGEKVRVLAYTNAYKVELFLNGESLGERSYENKKTSWGAPYKETKDGKTYLEWAVPFKAGKLEAVAKDENGNEIGRDQEVTAGEPAAVKLTADRKVISADGTDLSFITAEIVDNKGVVVPNADHLIKFNVSGQGELAGVDNGNASSVERYKDTKRKAFSGKALAIIQSNKLSGEITVNATAAGLSSDSTSLFTVSPADRDKKTAVGMDAVHMTVAVNEAPKLPSAIQVYYSDTSVDSKNVTWDEIDPKRYSSVGTFTVEGSVEGTSLKAKAFITVKGIVAVKPVSTATKIGVPPVLPEKVTLLYSDETTKEVNVTWEEILEDKLAKVGRFTVEGTVAETDSKAKVYVRVTNETKAVNIMLPAQGSSYPQLEATFTNPADNLQHLNDGIKSYTNSPVNRWTNWTRTPRDAGDSIIVNFGKTHVINNLDLFVFTDSGTVVPDKAAVQYWDGTAWKDVENLTQPSPYVVEKNEIRFDSVQTDKLKFHLAPSVKGKFLALTEAEVYADQIVMSSTAELSSMTVNGKALADFDAAKKNYELVLPYGSELPKIEAEAKDNATVTILPAFAYPGSAKLFVTSEDGQVTSEYSIQVSMEEPKLVSAELSANRTNLLEDDMIDLKVKGLFESKEIVDLTDSQTTYEFDKPIVKIEGNKLYALEAGEVKVKAAVTYKGVSVATPAVTFKIAKNPAPKTI

SEQ ID NO: 15 - protein, 1304 aa, Bifidobacterium mongoliense
VEDATRSDSTTQMSSTPEVVYSSAVDSKQDRTSDFDANWKFMLSDSVQAQDPTFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGEENTIVVKVENRLPSSRWYSGSGIYRDVTLTVTDGVHVGNNGVAIKTPSLATQNGGDVTMNLTTKVANDTKAAANITLKQTVFPKGGKTDAAIGTVTTASKSIAAGASADVTSTITAASPKLWSIKNPNLYTVRTEVLNGDTVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQTIAGDNAVLGGDKDETWAKFDLTSTINRDRNAPSVIMWSLGNEMMEGISGSVSDFPATSAKLVAWTKAADSTRPMTHGDNKIKANWNESNTMGDNLTANGGVVGTNYSDGANYDKIRKNHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKDSGNNVPVVVYTDAAKVKLYFTPKGSTEKRLIGEKSFTKKTTAAGYTYQVYEGANKNSTVHKNMYLTWNVPWAEGTISAEAYDENNKLIPEGSTEGNASVTTTGEAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSATTPVGTPAVLPGSRPAVLPDGTVTSANFAVDWTKPADTVYNTAGTVKVPGTATVFGKEFKVTATIRVQRSQVTIGSSVSGNALRLTQNIPADKQSDTLNAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNDTKVSDSVLAAGSYNTPAIIADVKAEGEGNASVTVLPAHDNVIRVITESEDHVTRKTFTINLGTEQEFPADSDERD

SEQ ID NO: 16 - protein, 27 aa, Bacillus clausii
MKKPLGKIVASTALLISVAFSSSIASA

SEQ ID NO: 17 - protein, 8 aa, Synthetic construct
HHHHHHPR

SEQ ID NO: 18 - protein, 10 aa, Synthetic construct
  VARIANT 3: note = X = any amino acid
  VARIANT 4: note = X = any amino acid
  VARIANT 7: note = X = I or L or R
  VARIANT 10: note = X = P or S or A
WTXXDYXGEX

SEQ ID NO: 19 - protein, 11 aa, Synthetic construct
  VARIANT 3: note = X =W or Y or F
  VARIANT 9: note = X=any amino acid
  VARIANT 10: note = X =Y or G
SRXYSGSGXXR

SEQ ID NO: 20 - skipped

SEQ ID NO: 21 - protein, 8 aa, Synthetic construct
  VARIANT 3: note = X=any amino acid
  VARIANT 5: note = X=A or S or D or T
  VARIANT 8: note = X=N or T or L
EYXHXMGX

SEQ ID NO: 22 - protein, 9 aa, Synthetic construct
  VARIANT 1: note = X=I or V
  VARIANT 3: note = X=any amino acid
  VARIANT 4: note = X=A or C or S
  VARIANT 8: note = X=N or P or D or Q or T or S
  VARIANT 9: note = X=D or H or Q or V
XRXXHYPXX

SEQ ID NO: 23-24 - skipped (2 entries)

SEQ ID NO: 25 - protein, 1032 aa, unidentified
NGYPEWNNNPDIFQLNRLEAHATLMPYKTLSEALNGKRHLSDFYQSLNGKWKFYFATNPEKREKNFYEMDYDYSDWDEMTVPGHWQLQGYDYPQYTNIRYPWELTEDIKPPFAPTKYNPVGQYIRTFTIPEDWNGMPVYISFQGVESAFYVWVNGDFVGYSEDSFTPAEFDLTPYLKEGENKLAVEVYRWSDASWLEDQDFWRLSGIFRDVYLYATPIVHIKDFFVQTDLDHDYKDAELKINVKISNYQKFYNNGDVVFEALLYDKNQQMILDKPLTKEIEIKNKENVEFNISSFVKNPLKWSAEKPNLYTLVLCLKNDDGTVIEYESCKIGFRKFEIKNGLMLINGKRIVFKGVNRHEFSHEKGRAIGYEEMVHDIKLMKKHNINAVRTSHYPNQPEFYDLCDEFGLYVIDETNLETHGTWRYGQEEEEDTIPGSKPEWAENVLDRCNSMFQRDKNHPCILIWSLGNESFGGDNFIKMHDFLRNADPTRIVHYEGVFHYRKSEAASDIESTMYTPPDLVEQYAIEAERSQNGAKPYLLCEYSHSMGNSNGNLYKYTDLFDKYPILQGGFIWDWKDQALKSKTKDGVEFLAYGGDFGDEPNDGNFCGNGLIFADGTITPKIFEVKKCYQNVDFKEIDLQKGIISVKNKFLFTNLRDFDFHWEIAEDGIKIKSGKHELDIEPGETKEIKLEYNMNELSGCKGELILTVSLRLKNSTSWADQGHEIAFEQFLLKEKQQIEIQENDSKNISWEEKDENVTVIGEDYCITFDKQNGNLVSYVSHGKELLKTPIVPNFWRALTDNDRGNKLGERCAPWKNAGEKRKLQDFAIVNDENRLNIKAEYILPDANDSKCEITYTIDALGSIVVDFQLVPNGNLPEIPEIGVMFSMPSEYDVLHWYGKGPHENYIDRNKGAKIGLYHGLVKDQYVPYLKPQECGNKTGVRWGTITNKEGFGLKVEGLPTVELNVLPYTPSELENCSHSYKLPQIDKTVVRINYKQMGVGGDDSWGQKTHPEFTIFAEKPYCYSFKLIPVNKK

SEQ ID NO: 26 - protein, 1032 aa, Unidentified
NGYPEWNNNPDIFQLNRLEAHATLMPYKTLSEALNGKRHLSDFYQSLNGKWKFYFATNPEKREKNFYEMDYDYSDWDEITVPGHWQLQGYDYPQYTNIRYPWELTEDIKPPFAPTKYNPVGQYIRTFTIPEDWNGMPVYISFQGVESAFYVWVNGDFVGYSEDSFTPAEFDLTPYLKEGENKLAVEVYRWSDASWLEDQDFWRLSGIFRDVYLYATPIVHIKDFFVQTDLDHDYKDAELKINVKISNYQKFYNNGDVVFEALLYDKNQQMILDKPLTKEIEIKNKENVEFNISSFVKNPLKWSAEKPNLYTLVLCLKNDDGTVIEYESCKIGLRKFEIKNGLMLINGKRIVFKGVNRHEFSHEKGRAIGYEEMVHDIKLMKKHNINAVRTSHYPNQPEFYDLCDEFGLYVIDETNLETHGTWRYGQEEEEDTIPGSKPEWAENVLDRCNSMFQRDKNHPCILIWSLGNESFGGDNFIKMHDFLRNADPTRIVHYEGVFHYRKSEAASDIESTMYTPPDLVEQYAIEAEKSQNGAKPYLLCEYSHSMGNSNGNLYKYTDPFDKYPILQGGFIWDWKDQALKSKTKDGVEFLAYGGDFGDEPNDGNFCGNGLIFADGTITPKIFEVKKCYQNVDFKEIDLQKGIISVKNKFLFTNLRDFDFHWEIAEDGIKIKSGKHELDIEPGETKEIKLEYNMNELSGCKGELILTVSLRLKNSTSWADQGHEIAFEQFLLKEKQQIEIQENDSKNISWEEKDENVTVIGEDYCITFDKQNGNLVSYVSHGKELLKTPIVPNFWRALTDNDRGNKLGERCAPWKNAGEKRKLQDFAIVNDENRLNIKAEYILPDANDSKCEITYTIDALGSIVVDFQLVPNGNLPEIPEIGVMFSMPSEYDVLHWYGKGPHENYIDRNKGAKTGLYHGLVKDQYVPYLKPQECGNKTGVRWGTITNKEGFGLKVEGLPTVELNVLPYTPSELENCSHSYKLPQIDKTVVRINYKQMGVGGDDSWGQKTHPEFTIFAEKPYCYSFKLIPVNKK

SEQ ID NO: 27 - protein, 1026 aa, Unidentified
MDHQNDWENPKILFKNRQPAHCTFIPRLNPFNAEWEYWPNYICLNGSWKFHWSPNPESRPVDFYRLDYDTSSWDEIEVPSNWEFQGYDIPIYTNVVYPFPANPPYIEHTRNSVGSYKRTFNIPEDWKDKEIFLHFAGVKSAFYVWVNGKEVGYSQGSCTPAEFDITDFVKPGENVLAVEVYRWCDGSYLEDQDMWWTSGIYRDVFIYATEKLHIRDYFIRNDFDEKYEDTILKIDVNVINYSDSTSEDLKLEVSLIDPEGRYCFEEPIVERIEKINPKEEKIVNIEKWVKEPKKWSAEKPNLYVVEMKLLNSEGEEIEVQKCNIGFRKVEIKEGQLLINGKAIKIKGVNRHEFDPDRGYAITVERMIQDIKLMKQYNINTVRTSHYPNDPKWYHLCDYFGMYLIDEANIESHGMGYDPERTLANNPEWKEAHLDRTIRMVERDKNHPSVIIWSLGNEAGDGINFEATSEWIHNRDKTRPVHYERAEFRPHVDIVSFMYTPMELLEEYAQREKERPVFMCEYAHAMGNSVGNLKDYWDLIKRHRNLLGGCIWDWVDQGIRKVDEEGREYWAYGGDFGDEPNDGNFCINGVVLPDRTPEPELEEVKKVYQYIEIEAVDLKKGRFRVTNNYYFTNLKEFDCYFELQEDGKKILEEKIGNIDLNPGESTEIEVPFENIDFKPDCEYFATIYFALKNDTSWSSRGYVVAWEQFKLPFGNSNFPRINLQNYPELVLTETTDNIKVKTANSQITLNKTTGMIESFRFMEKELILSEFRPNFWRVPIDNDIGNDMPERLGVWKEAGKELKILSNRIEKFDDKVVRIEFNGQLPKAENSDYKVNYTLFSSGDVILEIKLKANKKLPELPRFGLQGKISGDFRNVIWYGRGPHENYWDRKTGAKIGLYNCSVEDLIHMYVRPQETGNRSDVRWFKLTNQYGLNLMVIGMPFIDFSAWPFEMEDLEKANHINELNFKDLITLNIDYKQMGVGGDNSWGALTHPEYTLPSGEYFYSFRINVFKTGQECEGEILKRSFE

SEQ ID NO: 28 - protein, 1015 aa, Unidentified
QTFNEWKDPGVCQTGKVEPHAWFIPFESSELALKGNYEESDFITLLNGMWKFKFLTHPDSMIQDFYKPVYNVSQWNEIKVPADWQTEGYDYPIYTNIQYPFGNPTPPLISSDYNPTGLYRTTFELPQSWEGRKTFLLFGSVKTAFYLWINGEYVGYSEDSKTPAEFDITKFVKRGDNTLAMMVIRWCDASYLEDQDFWRLSGIERDVFLISTPQSRIKDFRVVSSLDGNYKNGIFNLDVQLESSSYDDLKVNCKLIDGKTTIFEKSLLPDKNGNVSFESVINNVKKWSAEKPYLYDLLIELEQDGKTLQATSIDVGFRKVEIKNGQLHVNGQPVYIRGVNLHEHHPVTGHVVDRETRLKDIMLMKRHNINTVRTSHYPQDPYFYELCDRYGMYVIDEANIESHGMGYRPDRTLANNPEWLEAHMYRTRNMVKRDKNHPSIIIWSLGNEAGNGYNTHQTYLWIKSVDTTRPVTYERAELEFNTDIYCPMYARMEDVENYARNHNDRPLIQCEYAHAMGNSVGNLQDYWDLIYKYPVLQGGCIWDWVDQGLYKTDENGNKFFAYGGDFGPENVPSDGNFCLNGLVNPDRKPHPSLYEVKKVYQPFYFKPVDLKNGKVEIINYNSFTNLNEYAVRYYIKADGKEIFRSKNLTISLNPLEKKTVTFDIPKIKPAINTEYFLFIEVINKTEKNLIPEGHIVAAEQFKLPYELTSPLVFNTAGKINVEENGNRINLGCDKIQIVIDKNDGFITSYELNGKEMFEIPLQPDFWRAPTDNDYGNRMQQRCKVWKNISEKFELKRIEWSQPDEGIVKVKCEYEIKGLGDWRSQRYRIASIDYTVYYDGTIKVNSKFNLNDKSLPEIPRIGFKTRLPGDMINLEYFGRGPHENYCDRKTSAFVDLYKSTVYDQYYPYIRPQETGYKTDVRWVSLKGKTDGKGLKVLGEPLLSTSALPFATEDLDEGDNKHNRHYIELKPRDFVEWHIDLKQMGVGGDNSWGARPHDEYMIFPGIYDFSFIIQPIK

SEQ ID NO: 29 - protein, 1053 aa, Unidentified
FINNPWEDAGITSINRDRARAVFCSFNSVSDALKYYPEYCTRIKYLNGIWKFFHVSLSDSSAIPDDFNILYHNEWNHIKVPSNWELQGYDFPIYKSSCYPFRPVIPPYIPLDDNSIGIYQRYFEIPAHWKGMNLTLRFEGVATAFKIWINGEFVGYGEDSFLSSEFNITPYLKPNDKNLLVVEVIRWGDGAYPEDQDHWRLSGIFRDVMIIAEPRLRIADIHWQAELDSIYSTAVLKIRPRLDNFTGQRISGYKLSAFLFDKEKKQVCKTMTCDASSVLYEIYPRLDNVKFAVMEDTIANIKLWSDEDPYLYTLVIALEDSVGNLLEAKSTRVGFRKIEFSRTNSKLLINGKETMLYGINRHDHHLVRGKALTHEDIRKDLITLKQFNFNCIRTSHYPNHPYFYDLCDELGFLVIDEANLETHGLGGKLSNDPLWTAAFMERVTRMVERDKNHPSVIMWSLGNESGRGPNHAAMAEWVHDFDITRFVHYEPAQGNHRVKGYIPPGSPGYPKDHAHRIQVPVDQYYVDVISRFYPGIFTPALLVNQPGDNRPIFFSEYAHSMGNSTGNLKEFWDIFRALPRIIGGCIWDFKDQGILKTDSAGNKFYAYGGDFGEKLHNGNFCINGIVDPDGRPKPAIYECKRVFQPVECVLIDTTDVKIKIINRHAETSLQRYSIYIQLLENGFLKMEKKLPSINTKAGCDTIMVFKSLLPDFKDGKEYLLKISFRLKNDMPWANRDFEVAANQFALTSLPRLDFKSNYDGLKLNLSENDTLFVISDTKFRVIFSKNSGYISEYTLNGIKYIIKPLKPDFTRPLTDNDRRGWKPYKKLKEWYEAVPRLKKFDVIAKNGDSIIITSDYSVIKDKADLFLKYTIYPGGILKVNYFLDIIDTTLPDIPAIGMQCGISNNLGMVKFYGRGPHENYIDRRYGADVGIYEMTVEEMNEPYIMPQETGNRTDVRWMTLYNRNNDNQKLTIKADSLLSMSVWPWTQENINKARHTNELVNPGYLTLNIDLIQMGVGGNDSWSDVSRPLSQYLIPARDYQYSFEICAQCILKK

SEQ ID NO: 30 - protein, 1023 aa, Unidentified
QPYWENPNIISENKEPYRAFFIPYKSAEKAKNLESSERVMLLNGIWKFHWSPDPESRPKDFYKESFNADGWDNIKVPGNWETQGYGVPIYTNITYPFKKDPPSVTSEPPENYTSYKHRNPVGSYIYYFTIDSMPAENVFLKFDGVSSAFFVWLNGKYIGYSENSMSPAEFNITGSINKGKNKLAVEVYRWCDGSYLEDQDMWRLSGIFRDVSLIFRPDIFVKDFQIITDLDKNYKNANLTVNTQIFAINPKKNSSINIYFEIFKPSELDRVLYQFSKKNLPVNNNHLNIGITEQINSPLLWTNETPNLYLLIIRLTDNNDRTLEIIPWYFGFREVELKNKQLYVNGVPIKIRGVNRHEHHPRTGRHVDLQTMELDIKLMKQCNVNLVRNSHYPNDYRWYMLCDRYGMFVMDEANQESHAYGIGNRKMGDDPIWEKSHVDRALSMVKTNKNFTGIIMWSLGNEGGSGRNITAMRKALLEVDTTRPVFYHADETNSDFRDADYPYPQELLKIAYSDTSKPTVMREYAHAMGNSLGNLKEFWDIIYAHRIITGGCIWDWVDQGLAKKINENKMSYPENETVLKKGDDEMWAYGGDFGDFPNDGNFCINGLLAPDRTPNPHYFEAQKIYQPIAMYPVNLTKGEIKIENKYYFTSLDNLTPRIIILENGHEILRKNQDPLKINPFKSGILKLPLNEITMNENHEYIARIEFILPENTLWADKGTVIAYDEFILSPYKFKKIYINTGEKITYKETTNDIQLSGNNLTVVLNKQNGNLTSLKKFKEEFIINPLEPYFWKPPNDNQLRNDYVKRTGKWKEVIQNRKVKHYIINPNEDGSISIMFDYGLAFETVSLKLEYTVYPDGKIKVLMDYYNEDNSLPLIPKFGIRMAVPSRYSPITWYGKGPYENYPDRNNGYPIGIYQLPVENFISNYIRPQDNANRTETRWFKLENKDYTIKIEGPQPINFRVWPYTEDDLERAKHPHELPHRDFLNVNIDYKIHGVGGTNSWGKKTLEKYTINPLGELNYGFII